(19) United States
(12) Patent Application Publication
(10) Pub. No.: US 2025/0066302 A1
(43) Pub. Date: Feb. 27, 2025

(54) DEUTERATED FORMS AND DERIVATIVES OF VOLINANSERIN

(71) Applicant: Terran Blosciences Inc, Miami Beach, FL (US)

(72) Inventors: Scott Weintraub, Lexington, MA (US); Scott L. HARBESON, Lexington, MA (US)

(21) Appl. No.: 18/941,426

(22) Filed: Nov. 8, 2024

Related U.S. Application Data
(63) Continuation of application No. 17/416,011, filed on Jun. 18, 2021.
(60) Provisional application No. 62/784,056, filed on Dec. 21, 2018.

(51) Int. Cl. C07D 211/22; C07B 59/00

(52) U.S. Cl. CPC C07D 211/22; C07B 59/002; C07B 2200/05

(57) ABSTRACT

Deuterated forms of volinanserin according to structural formula (I), and their pharmaceutically acceptable salts, pharmaceutical compositions containing these compounds, and methods of treatment or prevention using these compounds or pharmaceutical compositions are described. The compounds are useful for treating or preventing a disease or condition selected from psychosis, schizophrenia, schizoaffective disorder, Parkinson's disease, Lewy body dementia, sleep disorder (including insomnia), agitation, mood disorder (including depression), thromboembolic disorder, autism, and attention deficit hyperactivity disorder.

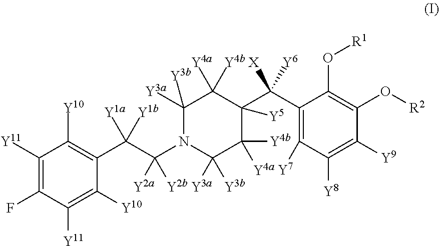

### RELATED APPLICATIONS

This application claims the benefit of U.S. Provisional Application No. 62/784,056, filed on Dec. 21, 2018. The entire teachings of this application are incorporated herein by reference.

### BACKGROUND OF THE INVENTION

Many current medicines suffer from poor absorption, distribution, metabolism and/or excretion (ADME) properties that prevent their wider use or limit their use in certain indications. Poor ADME properties are also a major reason for the failure of drug candidates in clinical trials. While formulation technologies and prodrug strategies can be employed in some cases to improve certain ADME properties, these approaches often fail to address the underlying ADME problems that exist for many drugs and drug candidates. One such problem is rapid metabolism that causes a number of drugs, which otherwise would be highly effective in treating a disease, to be cleared too rapidly from the body. A possible solution to rapid drug clearance is frequent or high dosing to attain a sufficiently high plasma level of drug. This, however, introduces a number of potential treatment problems such as poor patient compliance with the dosing regimen, side effects that become more acute with higher doses, and increased cost of treatment. A rapidly metabolized drug may also expose patients to undesirable toxic or reactive metabolites.

Another ADME limitation that affects many medicines is the formation of toxic or biologically reactive metabolites. As a result, some patients receiving the drug may experience toxicities, or the safe dosing of such drugs may be limited such that patients receive a suboptimal amount of the active agent. In certain cases, modifying dosing intervals or formulation approaches can help to reduce clinical adverse effects, but often the formation of such undesirable metabolites is intrinsic to the metabolism of the compound.

In some select cases, a metabolic inhibitor will be co-administered with a drug that is cleared too rapidly. Such is the case with the protease inhibitor class of drugs that are used to treat HIV infection. The FDA recommends that these drugs be co-dosed with ritonavir, an inhibitor of cytochrome P450 enzyme 3A4 (CYP3A4), the enzyme typically responsible for their metabolism (see Kempf, D. J., et al., Antimicrobial agents and chemotherapy, 1997, 41(3): 654-60). Ritonavir, however, causes adverse effects and adds to the pill burden for HIV patients who must already take a combination of different drugs. Similarly, the CYP2D6 inhibitor quinidine has been added to dextromethorphan for the purpose of reducing rapid CYP2D6 metabolism of dextromethorphan in a treatment of pseudobulbar affect. Quinidine, however, has unwanted side effects that greatly limit its use in potential combination therapy (see Wang, L., et al., Clinical Pharmacology and Therapeutics, 1994, 56(6 Pt 1): 659-67; and FDA label for quinidine at www.accessdata.fda.gov).

In general, combining drugs with cytochrome P450 inhibitors is not a satisfactory strategy for decreasing drug clearance. The inhibition of a CYP enzyme's activity can affect the metabolism and clearance of other drugs metabolized by that same enzyme. CYP inhibition can cause other drugs to accumulate in the body to toxic levels.

A potentially attractive strategy for improving a drug's metabolic properties is deuterium modification. In this approach, one attempts to slow the CYP-mediated metabolism of a drug or to reduce the formation of undesirable metabolites by replacing one or more hydrogen atoms with deuterium atoms. Deuterium is a safe, stable, non-radioactive isotope of hydrogen. Compared to hydrogen, deuterium forms stronger bonds with carbon. In select cases, the increased bond strength imparted by deuterium can positively impact the ADME properties of a drug, creating the potential for improved drug efficacy, safety, and/or tolerability. At the same time, because the size and shape of deuterium are essentially identical to those of hydrogen, replacement of hydrogen by deuterium would not be expected to affect the biochemical potency and selectivity of the drug as compared to the original chemical entity that contains only hydrogen.

Over the past 35 years, the effects of deuterium substitution on the rate of metabolism have been reported for a very small percentage of approved drugs (see, e.g., Blake, M I, et al., J Pharm Sci, 1975, 64:367-91; Foster, AB, Adv Drug Res 1985, 14:1-40 (“Foster”); Kushner, D J, et al., Can J Physiol Pharmacol 1999, 79-88; Fisher, M B, et al., Curr Opin Drug Discov Devel, 2006, 9:101-09 (“Fisher”)). The results have been variable and unpredictable. For some compounds, deuteration caused decreased metabolic clearance in vivo. For others, there was no change in metabolism. Still others demonstrated increased metabolic clearance. The variability in deuterium effects has also led experts to question or dismiss deuterium modification as a viable drug design strategy for inhibiting adverse metabolism (see Foster at p. 35 and Fisher at p. 101).

### SUMMARY OF THE INVENTION

This invention relates to deuterated forms and derivatives (including prodrugs) of volinanserin, and pharmaceutically acceptable salts thereof. In one aspect, the invention provides a compound of structural formula (I):

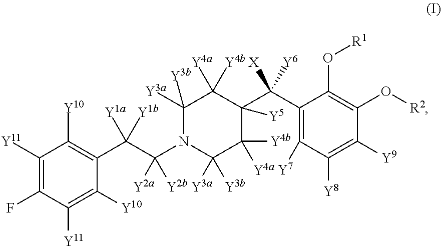

or a pharmaceutically acceptable salt thereof, wherein R¹ and R² are independently selected from —CH₃, —CH₂D, —CHD₂, and —CD₃; X is —OH or —F; and Y^{1a}, Y^{1b}, Y^{2a}, Y^{2b}, Y^{3a}, Y^{3b}, Y^{4a}, Y^{4b}, Y⁵, Y⁶, Y⁷, Y⁸, Y⁹, Y¹⁰, and Y¹¹ are each independently selected from hydrogen and deuterium;
provided that at least one of Y^{1a}, Y^{1b}, Y^{2a}, Y^{2b}, Y^{3a}, Y^{3b}, Y^{4a}, Y^{4b}, Y⁵, Y⁶, Y⁷, Y⁸, Y⁹, Y¹⁰, Y¹¹, R¹ and R² comprises deuterium; provided that when Y^{1a}, Y^{1b}, Y^{2a}, and Y^{2b} are each deuterium, then at least one of Y^{3a}, Y^{3b}, Y^{4a}, Y^{4b}, Y⁵, Y⁶, Y⁷, Y⁸, Y⁹, Y¹⁰, Y¹¹, R¹ and R² comprises deuterium; and provided that when Y^{3a}, Y^{3b}, Y^{4a}, and Y^{4b} are each deuterium, then at least one of Y^{1a}, Y^{1b}, Y^{2a}, Y^{2b}, Y⁵, Y⁶, Y⁷, Y⁸, Y⁹, Y¹⁰, Y¹¹, R¹ and R² comprises deuterium.

This invention also provides compositions comprising a compound of this invention, including pharmaceutical compositions comprising a compound, or pharmaceutically acceptable salt thereof, of this invention and a pharmaceutically acceptable carrier. This invention also provides the use of such compounds, salts and compositions in methods of treating diseases and conditions that are beneficially treated by administering volinanserin or other drugs whose principal effects are mediated by serotonin 2A (5-HT_{2A}) receptor inverse agonism or antagonism. Some exemplary embodiments include a method of treating or preventing a disease or condition selected from psychosis, schizophrenia (including chronic schizophrenia), schizoaffective disorder, Parkinson's disease (including Parkinson's disease psychosis), Lewy body dementia, sleep disorder (including insomnia), agitation, mood disorder (including depression), thromboembolic disorder, autism, attention deficit hyperactivity disorder, and any combination thereof, the method comprising the step of administering to a subject in need thereof a pharmaceutically acceptable compound, salt or composition of the present invention.

### DETAILED DESCRIPTION OF THE INVENTION

Volinanserin, also known as (R)-(+)-α-(2,3-dimethoxyphenyl)-1-[2-(4-fluorophenyl)ethyl]-4-piperidinemethanol, is a highly selective 5-HT_{2A} receptor antagonist. It is widely used in scientific research to investigate the function of the 5-HT_{2A} receptor.

Volinanserin was being investigated in clinical trials as a potential antipsychotic, antidepressant and treatment for insomnia, and is also active in animal models involving blockade of NMDA glutamatergic channel receptors, an effect known to resemble some behavioral symptoms of schizophrenia in man. De Paulis T, Curr Opin Investig Drugs. 2001 January; 2(1):123-32.

Despite the beneficial activities of volinanserin, there is a continuing need for new compounds to treat the aforementioned diseases and conditions.

### Definitions

The term “treat” means decrease, suppress, attenuate, diminish, arrest, or stabilize the development or progression of a disease (e.g., a disease delineated herein), lessen the severity of the disease or improve the symptoms associated with the disease.

“Disease” means any condition or disorder that damages or interferes with the normal function of a cell, tissue, or organ.

As used herein, the term “subject” includes humans and non-human mammals. Non-limiting examples of non-human mammals include mice, rats, guinea pigs, rabbits, dogs, cats, monkeys, apes, pigs, cows, sheep, horses, etc.

“The term “alkyl” refers to a monovalent saturated hydrocarbon group. Cₐ-C_{b} alkyl is an alkyl having from a to b carbon atoms. For example, C₁-C₆ alkyl is an alkyl having from 1 to 6 carbon atoms. In some embodiments, an alkyl may be linear or branched. In some embodiments, an alkyl may be primary, secondary, or tertiary. Non-limiting examples of alkyl groups include methyl; ethyl; propyl, including n-propyl and isopropyl; butyl, including n-butyl, isobutyl, sec-butyl, and t-butyl; pentyl, including, for example, n-pentyl, isopentyl, and neopentyl; and hexyl, including, for example, n-hexyl and 2-methylpentyl. Non-limiting examples of primary alkyl groups include methyl, ethyl, n-propyl, n-butyl, n-pentyl, and n-hexyl. Non-limiting examples of secondary alkyl groups include isopropyl, sec-butyl, and 2-methylpentyl. Non-limiting examples of tertiary alkyl groups include t-butyl.

The term “alkenyl” refers to a monovalent unsaturated hydrocarbon group where the unsaturation is represented by a double bond. C₂-C₆ alkenyl is an alkenyl having from 2 to 6 carbon atoms. An alkenyl may be linear or branched. Examples of alkenyl groups include CH₂═CH— (vinyl), CH₂═C(CH₃)—, CH₂═CH—CH₂— (allyl), CH₃—CH═CH—CH₂— (crotyl), CH₃—CH═C(CH₃)— and CH₃—CH═CH—CH(CH₃)—CH₂—. Where double bond stereoisomerism is possible, the stereochemistry of an alkenyl may be (E), (Z), or a mixture thereof.

The term “alkynyl” refers to a monovalent unsaturated hydrocarbon group where the unsaturation is represented by a triple bond. C₂-C₆ alkynyl is an alkynyl having from 2 to 6 carbon atoms. An alkynyl may be linear or branched. Examples of alkynyl groups include HC≡C—, CH₃—C≡C—, CH₃—C≡C≡CH₂—, CH₃—C≡C≡CH₂—CH₂— and CH₃—C≡C≡CH(CH₃)—CH₂—.

The compounds described herein can be PEGylated. A “PEGylated” compound refers to a compound that has at least one poly(ethylene glycol) chain covalently bound to it. For example, R³ of structural formula (II), described below, can be a poly(ethylene glycol) (PEG) group. Typically, the poly(ethylene glycol) can have a plurality of (e.g., n) repeat units (e.g., —O(CH₂CH₂O)ₙH with n between 5 and 350). The polyethylene glycol is not limited to any particular number of repeat units n or of any particular molecular weight, as long as the resulting PEGylated compound (e.g., of structural formula (II) described herein) is suitable as a prodrug. For example, the PEG group can have a molecular weight of up to 25 kDa. For example, the PEG group can be a low-molecular-weight PEG (i.e., ≤12 kDa), for example, having a molecular weight between 300 Da and 12 kDa, between 1 kDa and 12 kDa, between 3 kDa and 12 kDa, or between 3 kDa and 8 KDa. In another example, the PEG group can be a high-molecular-weight PEG (i.e., >12 kDa), for example, having a molecular weight between 12 kDa and 25 kDa, or between 18 kDa and 22 kDa.

“Amino acid ester” refers to those derivatives of an amino acid in which a carboxylic acid group is converted to an ester. For example, amino acid ester includes valine ester, leucine ester, isoleucine ester, alpha-t-butylglycine ester, dimethyl glycine ester, and the like.

Suitable amino acids include, but are not limited to, histidine (His), isoleucine (Ile), leucine (Leu), lysine (Lys), methionine (Met), phenylalanine (Phe), threonine (Thr), tryptophan (Trp), valine (Val), arginine (Arg), cysteine (Cys), glutamine (Gln), glycine (Gly), proline (Pro), serine (Ser), tyrosine (Tyr), alanine (Ala), asparagine (Asn), aspartic acid (Asp), glutamic acid (Glu), and selenocysteine (Sec).

It will be recognized that some variation of natural isotopic abundance occurs in a synthesized compound depending upon the origin of chemical materials used in the synthesis. Thus, a preparation of volinanserin will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Wada, E., et al., Seikagaku, 1994, 66:15; Gannes, L Z, et al., Comp Biochem Physiol Mol Integr Physiol, 1998, 119:725.

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural isotopic composition. However, in certain embodiments where stated, when a position is designated specifically as “H” or “hydrogen”, the position has at least 80%, at least 90%, at least 95%, at least 96%, at least 97%, at least 98%, or at least 99% hydrogen. In some embodiments where stated, when a position is designated specifically as “H” or “hydrogen”, the position incorporates ≤20% deuterium, ≤10% deuterium, 5% deuterium, ≤4% deuterium, ≤3% deuterium, ≤2% deuterium, or ≤1% deuterium. Also unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

The term “isotopic enrichment factor,” as used herein, means the ratio between the isotopic abundance and the natural abundance of a specified isotope.

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In some embodiments, in a compound of this invention, each designated deuterium atom has deuterium incorporation of at least 52.5%.

In some embodiments, in a compound of this invention, each designated deuterium atom has deuterium incorporation of at least 60%.

In some embodiments, in a compound of this invention, each designated deuterium atom has deuterium incorporation of at least 67.5%.

In some embodiments, in a compound of this invention, each designated deuterium atom has deuterium incorporation of at least 75%.

In some embodiments, in a compound of this invention, each designated deuterium atom has deuterium incorporation of at least 82.5%.

In some embodiments, in a compound of this invention, each designated deuterium atom has deuterium incorporation of at least 90%.

In some embodiments, in a compound of this invention, each designated deuterium atom has deuterium incorporation of at least 95%.

In some embodiments, in a compound of this invention, each designated deuterium atom has deuterium incorporation of at least 97.5%.

In some embodiments, in a compound of this invention, each designated deuterium atom has deuterium incorporation of at least 99%.

In some embodiments, in a compound of this invention, each designated deuterium atom has deuterium incorporation of at least 99.5%.

The term “isotopologue” refers to a molecule in which the chemical structure differs from a species of this invention only in the isotopic composition thereof.

The term “compound,” when referring to a compound of this invention, refers to a collection of molecules having an identical chemical structure, except that there may be isotopic variation among the constituent atoms of the molecules. Thus, it will be clear to those of skill in the art that a compound represented by a particular chemical structure will contain molecules having deuterium at each of the positions designated as deuterium in the chemical structure, and may also contain isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. In certain embodiments, the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

The invention also provides salts (e.g., pharmaceutically acceptable salts) of the compounds of the invention. Unless indicated otherwise and even through not explicitly stated, a salt (e.g., a pharmaceutically acceptable salt) of a compound described herein can be substituted for a compound described herein (e.g., a compound of structural formula (I), (II)) in any embodiment described herein, or aspect thereof.

A salt of a compound of this invention is formed between an acid and a basic group of the compound, such as an amino functional group, or a base and an acidic group of the compound, such as a carboxyl functional group. According to one embodiment, the compound is a pharmaceutically acceptable acid addition salt. In one embodiment the acid addition salt may be a deuterated acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, (3-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid. In one embodiment, the acids commonly employed to form pharmaceutically acceptable salts include the above-listed inorganic acids, wherein at least one hydrogen is replaced with deuterium.

The compounds of the present invention (e.g., compounds of Formula I), may contain an asymmetric carbon atom, for example, as the result of deuterium substitution or otherwise. As such, compounds of this invention can exist as either individual enantiomers, or mixtures of the two enantiomers. Accordingly, a compound of the present invention may exist as either a racemic mixture or a scalemic mixture, or as individual respective stereoisomers that are substantially free from another possible stereoisomer. The term “substantially free of other stereoisomers” as used herein means less than 25% of other stereoisomers, preferably less than 10% of other stereoisomers, more preferably less than 5% of other stereoisomers and most preferably less than 2% of other stereoisomers are present. Methods of obtaining or synthesizing an individual enantiomer for a given compound are known in the art and may be applied as practicable to final compounds or to starting material or intermediates.

Unless otherwise indicated, when a disclosed compound is named or depicted by a structure without specifying the stereochemistry and has one or more chiral centers, it is understood to represent all possible stereoisomers of the compound.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” and “d” both refer to deuterium. “Stereoisomer” refers to both enantiomers and diastereomers. “Tert” and “t-” each refer to tertiary. “Sec” or “s-” each refer to secondary. “n-” refers to normal. “i-” refers to iso. “US” refers to the United States of America.

“Substituted with deuterium” refers to the replacement of one or more hydrogen atoms with a corresponding number of deuterium atoms.

Throughout this specification, a variable may be referred to generally (e.g., “each R”) or may be referred to specifically (e.g., R¹, R², R³, etc.). Unless otherwise indicated, when a variable is referred to generally, it is meant to include all specific embodiments of that particular variable.

### Therapeutic Compounds

In certain embodiments, the present invention provides a compound of structural formula (I):

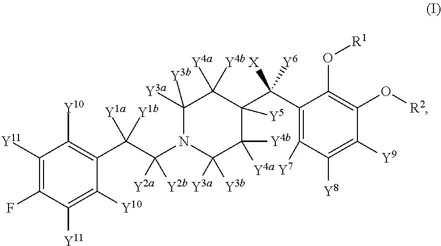

wherein:
R¹ and R² are independently selected from —CH₃, —CH₂D, —CHD₂, and —CD₃;
X is —OH or —F; and
Y^{1a}, Y^{1b}, Y^{2a}, Y^{2b}, Y^{3a}, Y^{3b}, Y^{4a}, Y^{4b}, Y⁵, Y⁶, Y⁷, Y⁸, Y⁹, Y¹⁰, and Y¹¹ are each independently selected from hydrogen and deuterium;
provided that at least one of Y^{1a}, Y^{1b}, Y^{2a}, Y^{2b}, Y^{3a}, Y^{3b}, Y^{4a}, Y^{4b}, Y⁵, Y⁶, Y⁷, Y⁸, Y⁹, Y¹⁰, Y¹¹, R¹ and R² comprises deuterium;
provided that when Y^{1a}, Y^{1b}, Y^{2a}, and Y^{2b} are each deuterium, then at least one of Y^{3a}, Y^{3b}, Y^{4a}, Y^{4b}, Y⁵, Y⁶, Y⁷, Y⁸, Y⁹, Y¹⁰, Y¹¹, R¹ and R² comprises deuterium; and provided that when Y^{3a}, Y^{3b}, Y^{4a}, and Y^{4b} are each deuterium, then at least one of Y^{1a}, Y^{1b} Y^{2a}, Y^{2b}, Y⁵, Y⁶, Y⁷, Y⁸, Y⁹, Y¹⁰, Y¹¹, R¹ and R² comprises deuterium.

In further embodiments, the present invention provides a compound of structural formula (I) wherein: R¹ and R² are independently selected from —CH₃, —CH₂D, —CHD₂, and —CD₃; X is —OH or —F; and Y^{1a}, Y^{1b}, Y^{2a}, Y^{2b}, Y^{3a} Y^{3b}, Y^{4a}, Y^{4b}, Y⁵, Y⁶, Y⁷, Y⁸, Y⁹, Y¹⁰, and Y¹¹ are each independently selected from hydrogen and deuterium; provided that at least one of Y^{1a}, Y^{1b}, Y^{2a}, Y^{2b}, Y^{3a}, Y^{3b}, Y^{4a}, Y^{4b}, Y⁵, Y⁶, Y⁷, Y⁸, Y⁹, Y¹⁰, Y¹¹, R¹ and R² comprises deuterium.

In further embodiments, the present invention provides a compound of structural formula (II) (i.e., a prodrug of the compound of structural formula (I)):

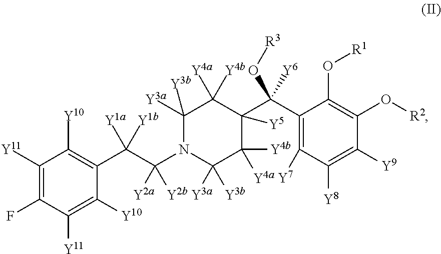

or a
pharmaceutically acceptable salt thereof, wherein:
R¹ and R² are independently selected from —CH₃, —CH₂D, —CHD₂, and —CD₃; R³ is —C(O)—C₁₋₂₁ alkyl (e.g., C(O)—C₁₋₆ alkyl, —C(O)—C₅₋₁₉ alkyl, —C(O)—C₉₋₁₇ alkyl, or —C(O)—C₁₅ alkyl (i.e., palmitoyl)), —C(O)—C₂₋₈ alkenyl, C(O)—C₂₋₈ alkynyl, polyethylene glycol (PEG), or an amino acid, wherein the amino acid is attached to the oxygen to which the R³ group is bonded through its carboxylic acid group thereby forming an amino acid ester; and Y^{1a}, Y^{1b}, Y^{2a}, Y^{2b}, Y^{3a}, Y^{3b}, Y^{4a}, Y^{4b}, Y⁵, Y⁶, Y⁷, Y⁸, Y⁹, Y¹⁰, and Y¹¹ are each independently selected from hydrogen and deuterium;
provided that at least one of Y^{1a}, Y^{1b}, Y^{2a}, Y^{2b}, Y^{3a}, Y^{3b}, Y^{4a}, Y^{4b}, Y⁵, Y⁶, Y⁷, Y⁸, Y⁹, Y¹⁰, Y¹¹, R¹ and R² comprises deuterium. In an aspect of this embodiment of the compound of structural formula (II) it is further provided that when Y^{1a}, Y^{1b}, Y^{2a}, and Y^{2b} are each deuterium, then at least one of Y^{3a}, Y^{3b}, Y^{4a}, Y^{4b}, Y⁵, Y⁶, Y⁷, Y⁸, Y⁹, Y¹⁰, Y¹¹, R¹ and R² comprises deuterium; and provided that when Y^{3a}, Y^{3b}, Y^{4a}, and Y^{4b} are each deuterium, then at least one of Y^{1a}, Y^{1b} Y^{2a}, Y^{2b}, Y⁵, Y⁶, Y⁷, Y⁸, Y⁹, Y¹⁰, Y¹¹, R¹ and R² comprises deuterium.

In a certain embodiment of the compound of structural formula (I) or the compound of structural formula (II), R¹ and R² are independently selected from —CH₃ and —CD₃. In an aspect of this embodiment, X, when present, is —OH. In a further aspect of this embodiment or any of the foregoing aspects of this embodiment, Y⁷, Y⁸, Y⁹, Y¹⁰, and Y¹¹ are each hydrogen. In a further aspect of this embodiment or any of the foregoing aspects of this embodiment, Y^{4a}, Y^{4b}, Y⁵, Y⁷, Y⁸, Y⁹, Y¹⁰, and Y¹¹ are each hydrogen. In yet a further aspect of this embodiment or any of the foregoing aspects of this embodiment, Y^{1a} and Y^{1b} are the same. In yet a further aspect of this embodiment or any of the foregoing aspects of this embodiment, Y^{2a} and Y^{2b} are the same. In yet a further aspect of this embodiment or any of the foregoing aspects of this embodiment, Y^{3a} and Y^{3b} are the same. In yet a further aspect of this embodiment or any of the foregoing aspects of this embodiment, Y^{4a} and Y^{4b} are the same.

In yet a further aspect of this embodiment, R¹ and R² are independently selected from —CH₃ and —CD₃; X, when present, is —OH; Y^{1a} and Y^{1b} are the same; Y^{2a} and Y^{2b} are the same; Y^{3a} and Y^{3b} are the same; Y^{4a} and Y^{4b} are the same; and Y⁷, Y⁸, Y⁹, Y¹⁰, and Y¹¹ are each hydrogen

In yet a further aspect of this embodiment, R¹ and R² are independently selected from —CH₃ and —CD₃; X, when present, is —OH; Y^{4a}, Y^{4b}, Y⁵, Y⁷, Y⁸, Y⁹, Y¹⁰, and Y¹¹ are each hydrogen; Y^{1a}, Y^{1b}, Y^{2a} and Y^{2b} are the same; and Y^{3a} and Y^{3b} are the same. In yet a further aspect of this embodiment, R¹ and R² are independently selected from —CH₃ and —CD₃; X, when present, is —OH; Y^{4a}, Y^{4b}, Y⁵, Y⁷, Y⁸, Y⁹, Y¹⁰, and Y¹¹ are each hydrogen; Y^{1a} and Y^{1b} are the same; and Y^{2a}, Y^{2b}, Y^{3a} and Y^{3b} are the same. In yet a further aspect of this embodiment or any of the foregoing aspects of this embodiment, Y^{2a} and Y^{2b} are deuterium. In yet a further aspect of this embodiment or any of the foregoing aspects of this embodiment, any atom not designated as deuterium is present at its natural isotopic abundance.

In another embodiment of the compound of structural formula (I) or the compound of structural formula (II), Y^{4a}, Y^{4b}, Y⁵, Y⁷, Y⁸, Y⁹, Y¹⁰, and Y¹¹ are each hydrogen. In yet a further aspect of this embodiment or any of the foregoing aspects of this embodiment, Y^{1a} and Y^{1b} are the same. In yet a further aspect of this embodiment or any of the foregoing aspects of this embodiment, Y^{2a} and Y^{2b} are the same. In yet a further aspect of this embodiment or any of the foregoing aspects of this embodiment, Y^{3a} and Y^{3b} are the same. In yet a further aspect of this embodiment, R¹ and R² are independently selected from —CH₃ and —CD₃; X, when present, is —OH; Y^{4a}, Y^{4b}, Y⁵, Y⁷, Y⁸, Y⁹, Y¹⁰, and Y¹¹ are each hydrogen; Y^{1a}, Y^{1b}, Y^{2a} and Y^{2b} are the same; and Y^{3a} and Y^{3b} are the same. In yet a further aspect of this embodiment, R¹ and R² are independently selected from —CH₃ and —CD₃; X, when present, is —OH; Y^{4a}, Y^{4b}, Y⁵, Y⁷, Y⁸, Y⁹, Y¹⁰, and Y¹¹ are each hydrogen; Y^{1a} and Y^{1b} are the same; and Y^{2a}, Y^{2b}, Y^{3a} and Y^{3b} are the same. In yet a further aspect of this embodiment or any of the foregoing aspects of this embodiment, Y^{2a} and Y² are deuterium. In yet a further aspect of this embodiment or any of the foregoing aspects of this embodiment, any atom not designated as deuterium is present at its natural isotopic abundance.

In another embodiment of the compound of structural formula (I) or the compound of structural formula (II), Y^{1a} and Y^{1b} are the same. In yet a further aspect of this embodiment, Y^{2a} and Y^{2b} are the same. In yet a further aspect of this embodiment or any of the foregoing aspects of this embodiment, Y^{3a} and Y^{3b} are the same. In yet a further aspect of this embodiment, R¹ and R² are independently selected from —CH₃ and —CD₃; X, when present, is —OH; Y⁴a Y^{4b}, Y⁵, Y⁷, Y⁸, Y⁹, Y¹⁰, and Y¹¹ are each hydrogen; Y^{1a}, Y^{1b}, Y^{2a} and Y^{2b} are the same; and Y^{3a} and Y^{3b} are the same. In yet a further aspect of this embodiment, R¹ and R² are independently selected from —CH₃ and —CD₃; X, when present, is —OH; Y^{4a}, Y^{4b}, Y⁵, Y⁷, Y⁸, Y⁹, Y¹⁰, and Y¹¹ are each hydrogen; Y^{1a} and Y^{1b} are the same; and Y^{2a}, Y^{2b}, Y^{3a} and Y^{3b} are the same. In yet a further aspect of this embodiment or any of the foregoing aspects of this embodiment, Y^{2a} and Y^{2b} are deuterium. In yet a further aspect of this embodiment or any of the foregoing aspects of this embodiment, any atom not designated as deuterium is present at its natural isotopic abundance.

In another embodiment of the compound of structural formula (I) or the compound of structural formula (II), Y^{2a} and Y² are the same. In yet a further aspect of this embodiment, Y^{3a} and Y^{3b} are the same. In yet a further aspect of this embodiment, R¹ and R² are independently selected from —CH₃ and —CD₃; X, when present, is —OH; Y^{4a}, Y^{4b}, Y⁵, Y⁷, Y⁵, Y⁹, Y¹⁰, and Y¹¹ are each hydrogen; Y^{1a}, Y^{1b}, Y^{2a} and Y^{2b} are the same; and Y^{3a} and Y^{3b} are the same. In yet a further aspect of this embodiment, R¹ and R² are independently selected from —CH₃ and —CD₃; X, when present, is —OH; Y^{4a}, Y^{4b}, Y⁵, Y⁷, Y⁸, Y⁹, Y¹⁰, and Y¹¹ are each hydrogen; Y^{1a} and Y^{1b} are the same; and Y^{2a}, Y^{2b}, Y^{3a} and Y^{3b} are the same. In yet a further aspect of this embodiment or any of the foregoing aspects of this embodiment, Y^{2a} and Y^{2b} are deuterium. In yet a further aspect of this embodiment or any of the foregoing aspects of this embodiment, any atom not designated as deuterium is present at its natural isotopic abundance.

In another embodiment of the compound of structural formula (I) or the compound of structural formula (II), Y^{3a} and Y^{3b} are the same. In yet a further aspect of this embodiment, R¹ and R² are independently selected from —CH₃ and —CD₃; X, when present, is —OH; Y^{4a}, Y^{4b}, Y⁵, Y⁷, Y⁸, Y⁹, Y¹⁰, and Y¹¹ are each hydrogen; Y^{1a}, Y^{1b}, Y^{2a} and Y^{2b} are the same; and Y^{3a} and Y^{3b} are the same. In yet a further aspect of this embodiment, R¹ and R² are independently selected from —CH₃ and —CD₃; X, when present, is —OH; Y^{4a}, Y^{4b}, Y⁵, Y⁷, Y⁵, Y⁹, Y¹⁰, and Y¹¹ are each hydrogen; Y^{1a} and Y^{1b} are the same; and Y^{2a}, Y^{2b}, Y^{3a} and Y^{3b} are the same. In yet a further aspect of this embodiment or any of the foregoing aspects of this embodiment, Y^{2a} and Y^{2b} are deuterium. In yet a further aspect of this embodiment or any of the foregoing aspects of this embodiment, any atom not designated as deuterium is present at its natural isotopic abundance.

In another embodiment of the compound of structural formula (I) or the compound of structural formula (II), R¹ and R² are independently selected from —CH₃ and —CD₃; X, when present, is —OH; Y⁴a Y^{4b}, Y⁵, Y⁷, Y⁸, Y⁹, Y¹⁰, and Y¹¹ are each hydrogen; Y^{1a}, Y^{1b}, Y^{2a} and Y^{2b} are the same; and Y^{3a} and Y^{3b} are the same. In yet a further aspect of this embodiment, Y^{2a} and Y^{2b} are deuterium. In yet a further aspect of this embodiment or any of the foregoing aspects of this embodiment, any atom not designated as deuterium is present at its natural isotopic abundance.

In another embodiment of the compound of structural formula (I) or the compound of structural formula (II), R¹ and R² are independently selected from —CH₃ and —CD₃; X, when present, is —OH; Y^{4a}, Y^{4b}, Y⁵, Y⁷, Y⁸, Y⁹, Y¹⁰, and Y¹¹ are each hydrogen; Y^{1a} and Y^{1b} are the same; and Y^{2a}, Y^{2b}, Y^{3a} and Y^{3b} are the same. In yet a further aspect of this embodiment, Y^{2a} and Y^{2b} are deuterium. In yet a further aspect of this embodiment or any of the foregoing aspects of this embodiment, any atom not designated as deuterium is present at its natural isotopic abundance.

In another embodiment of the compound of structural formula (I) or the compound of structural formula (II), Y^{2a} and Y^{2b} are deuterium. In yet a further aspect of this embodiment, any atom not designated as deuterium is present at its natural isotopic abundance.

In another embodiment of the compound of structural formula (I) or the compound of structural formula (II), any atom not designated as deuterium is present at its natural isotopic abundance. 1621 In some embodiments, the compound of structural formula (I) or structural formula (II) is selected from any one of the Compounds set forth in Table 1 (below), wherein X, when present, is —OH; Y^{1a} and Y^{1b} are the same; Y^{2a} and Y^{2b} are the same; Y^{3a} and Y^{3b} are the same; and Y^{4a}a Y^{4b}, Y⁵, Y⁷, Y⁸, Y⁹, Y¹⁰, and Y¹¹ are each hydrogen:

TABLE 1
Exemplary Embodiments of structural formula (I)

| Compound # | R¹ | R² | Y^{1a}/Y^{1b} | Y^{2a}/Y^{2b} | Y^{3a}/Y^{3b} | Y⁶ |
|---|---|---|---|---|---|---|
| 100 | CH₃ | CH₃ | H | H | H | D |
| 101 | CH₃ | CH₃ | H | H | D | H |
| 102 | CH₃ | CH₃ | H | H | D | D |
| 103 | CH₃ | CH₃ | H | D | H | H |
| 104 | CH₃ | CH₃ | H | D | H | D |
| 105 | CH₃ | CH₃ | H | D | D | H |
| 106 | CH₃ | CH₃ | H | D | D | D |
| 107 | CH₃ | CH₃ | D | H | H | H |
| 108 | CH₃ | CH₃ | D | H | H | D |
| 109 | CH₃ | CH₃ | D | H | D | H |
| 110 | CH₃ | CH₃ | D | H | D | D |
| 111 | CH₃ | CH₃ | D | D | H | H |
| 112 | CH₃ | CH₃ | D | D | H | D |
| 113 | CH₃ | CH₃ | D | D | D | H |
| 114 | CH₃ | CH₃ | D | D | D | D |
| 115 | CH₃ | CD₃ | H | H | H | H |
| 116 | CH₃ | CD₃ | H | H | H | D |
| 117 | CH₃ | CD₃ | H | H | D | H |
| 118 | CH₃ | CD₃ | H | H | D | D |
| 119 | CH₃ | CD₃ | H | D | H | H |
| 120 | CH₃ | CD₃ | H | D | H | D |
| 121 | CH₃ | CD₃ | H | D | D | H |
| 122 | CH₃ | CD₃ | H | D | D | D |
| 123 | CH₃ | CD₃ | D | H | H | H |
| 124 | CH₃ | CD₃ | D | H | H | D |
| 125 | CH₃ | CD₃ | D | H | D | H |
| 126 | CH₃ | CD₃ | D | H | D | D |
| 127 | CH₃ | CD₃ | D | D | H | H |
| 128 | CH₃ | CD₃ | D | D | H | D |
| 129 | CH₃ | CD₃ | D | D | D | H |
| 130 | CH₃ | CD₃ | D | D | D | D |
| 131 | CD₃ | CH₃ | H | H | H | H |
| 132 | CD₃ | CH₃ | H | H | H | D |
| 133 | CD₃ | CH₃ | H | H | D | H |
| 134 | CD₃ | CH₃ | H | H | D | D |
| 135 | CD₃ | CH₃ | H | D | H | H |
| 136 | CD₃ | CH₃ | H | D | H | D |
| 137 | CD₃ | CH₃ | H | D | D | H |
| 138 | CD₃ | CH₃ | H | D | D | D |
| 139 | CD₃ | CH₃ | D | H | H | H |
| 140 | CD₃ | CH₃ | D | H | H | D |
| 141 | CD₃ | CH₃ | D | H | D | H |
| 142 | CD₃ | CH₃ | D | H | D | D |
| 143 | CD₃ | CH₃ | D | D | H | H |
| 144 | CD₃ | CH₃ | D | D | H | D |
| 145 | CD₃ | CH₃ | D | D | D | H |
| 146 | CD₃ | CH₃ | D | D | D | D |
| 147 | CD₃ | CD₃ | H | H | H | H |
| 148 | CD₃ | CD₃ | H | H | H | D |
| 149 | CD₃ | CD₃ | H | H | D | H |
| 150 | CD₃ | CD₃ | H | H | D | D |
| 151 | CD₃ | CD₃ | H | D | H | H |
| 152 | CD₃ | CD₃ | H | D | H | D |
| 153 | CD₃ | CD₃ | H | D | D | H |
| 154 | CD₃ | CD₃ | H | D | D | D |
| 155 | CD₃ | CD₃ | D | H | H | H |
| 156 | CD₃ | CD₃ | D | H | H | D |
| 157 | CD₃ | CD₃ | D | H | D | H |
| 158 | CD₃ | CD₃ | D | H | D | D |
| 159 | CD₃ | CD₃ | D | D | H | H |
| 160 | CD₃ | CD₃ | D | D | H | D |
| 161 | CD₃ | CD₃ | D | D | D | H |
| 162 | CD₃ | CD₃ | D | D | D | D |

In some embodiments, the compound is selected from any one of the Compounds set forth in Table 1 (above), wherein any atom not designated as deuterium is present at its natural isotopic abundance.

In some embodiments, the compound of structural formula (I) or structural formula (II) is selected from any one of the Compounds set forth in Table 2 (below), wherein X, when present, is —OH; Y^{1a} and Y^{1b} are the same; Y^{2a} and Y^{2b} are the same; Y^{3a} and Y^{3b} are the same; and Y^{4a}, Y^{4b}, Y⁵, Y⁷, Y⁸, Y⁹, Y¹⁰, and Y¹¹ are each hydrogen:

TABLE 2
Exemplary Embodiments of structural formula (I)

| Compound # | R¹ | R² | Y^{1a}/Y^{1b} | Y^{2a}/Y^{2b} | Y^{3a}/Y^{3b} | Y⁶ |
|---|---|---|---|---|---|---|
| 131 | CD₃ | CH₃ | H | H | H | H |
| 115 | CH₃ | CD₃ | H | H | H | H |
| 147 | CD₃ | CD₃ | H | H | H | H |
| 132 | CD₃ | CH₃ | H | H | H | D |
| 116 | CH₃ | CD₃ | H | H | H | D |
| 148 | CD₃ | CD₃ | H | H | H | D |
| 139 | CD₃ | CH₃ | D | H | H | H |
| 123 | CH₃ | CD₃ | D | H | H | H |
| 155 | CD₃ | CD₃ | D | H | H | H |
| 135 | CD₃ | CH₃ | H | D | H | H |
| 119 | CH₃ | CD₃ | H | D | H | H |
| 151 | CD₃ | CD₃ | H | D | H | H |
| 137 | CD₃ | CH₃ | H | D | D | H |
| 121 | CH₃ | CD₃ | H | D | D | H |
| 153 | CD₃ | CD₃ | H | D | D | H |
| 138 | CD₃ | CH₃ | H | D | D | D |
| 122 | CH₃ | CD₃ | H | D | D | D |
| 154 | CD₃ | CD₃ | H | D | D | D |
| 145 | CD₃ | CH₃ | D | D | D | H |
| 129 | CH₃ | CD₃ | D | D | D | H |
| 161 | CD₃ | CD₃ | D | D | D | H |
| 146 | CD₃ | CH₃ | D | D | D | D |
| 130 | CH₃ | CD₃ | D | D | D | D |
| 162 | CD₃ | CD₃ | D | D | D | D |

In some embodiments, the compound is selected from any one of the Compounds set forth in Table 2 (above), wherein any atom not designated as deuterium is present at its natural isotopic abundance. 1661 In some embodiments, the compound of structural formula (I) or structural formula (II) is selected from any one of the Compounds set forth in Table 3 (below), wherein X is —OH; Y^{1a} and Y^{1b} are the same; Y^{2a} and Y^{2b} are the same; Y^{3a} and Y^{3b} are the same; Y⁴ and Y^{4b} are the same; and Y⁷, Y⁸, Y⁹, Y¹⁰, and Y¹¹ are each hydrogen:

TABLE 3
Exemplary Embodiments of structural formula (I)

| Compound # | R¹ | R² | Y^{1a}/Y^{1b} | Y^{2a}/Y^{2b} | Y^{3a}/Y^{3b} | Y^{4a}/Y^{4b} | Y⁵ | Y⁶ |
|---|---|---|---|---|---|---|---|---|
| 200 | CH₃ | CH₃ | H | H | H | D | D | D |
| 201 | CH₃ | CH₃ | H | H | H | D | H | D |
| 202 | CH₃ | CH₃ | H | H | H | H | D | D |
| 203 | CH₃ | CH₃ | H | H | D | D | D | H |
| 204 | CH₃ | CH₃ | H | H | D | D | H | H |
| 205 | CH₃ | CH₃ | H | H | D | H | D | H |
| 206 | CH₃ | CH₃ | H | H | D | D | D | D |
| 207 | CH₃ | CH₃ | H | H | D | D | H | D |
| 208 | CH₃ | CH₃ | H | H | D | H | D | D |
| 209 | CH₃ | CH₃ | H | D | H | D | D | H |
| 210 | CH₃ | CH₃ | H | D | H | D | H | H |
| 211 | CH₃ | CH₃ | H | D | H | H | D | H |
| 212 | CH₃ | CH₃ | H | D | H | D | D | D |
| 213 | CH₃ | CH₃ | H | D | H | D | H | D |
| 214 | CH₃ | CH₃ | H | D | H | H | D | D |
| 215 | CH₃ | CH₃ | H | D | D | D | D | H |
| 216 | CH₃ | CH₃ | H | D | D | D | H | H |
| 217 | CH₃ | CH₃ | H | D | D | H | D | H |
| 218 | CH₃ | CH₃ | H | D | D | D | D | D |
| 219 | CH₃ | CH₃ | H | D | D | D | H | D |
| 220 | CH₃ | CH₃ | H | D | D | H | D | D |
| 221 | CH₃ | CH₃ | D | H | H | D | D | H |
| 222 | CH₃ | CH₃ | D | H | H | D | H | H |
| 223 | CH₃ | CH₃ | D | H | H | H | D | H |
| 224 | CH₃ | CH₃ | D | H | H | D | D | D |
| 225 | CH₃ | CH₃ | D | H | H | D | H | D |
| 226 | CH₃ | CH₃ | D | H | H | H | D | D |
| 227 | CH₃ | CH₃ | D | H | D | D | D | H |
| 228 | CH₃ | CH₃ | D | H | D | D | H | H |
| 229 | CH₃ | CH₃ | D | H | D | H | D | H |
| 230 | CH₃ | CH₃ | D | H | D | D | D | D |
| 231 | CH₃ | CH₃ | D | H | D | D | H | D |
| 232 | CH₃ | CH₃ | D | H | D | H | D | D |
| 233 | CH₃ | CH₃ | D | D | H | D | D | H |
| 234 | CH₃ | CH₃ | D | D | H | D | H | H |
| 235 | CH₃ | CH₃ | D | D | H | H | D | H |
| 236 | CH₃ | CH₃ | D | D | H | D | D | D |
| 237 | CH₃ | CH₃ | D | D | H | D | H | D |
| 238 | CH₃ | CH₃ | D | D | H | H | D | D |
| 239 | CH₃ | CH₃ | D | D | D | D | D | H |
| 240 | CH₃ | CH₃ | D | D | D | D | H | H |
| 241 | CH₃ | CH₃ | D | D | D | H | D | H |
| 242 | CH₃ | CH₃ | D | D | D | D | D | D |
| 243 | CH₃ | CH₃ | D | D | D | D | H | D |
| 244 | CH₃ | CH₃ | D | D | D | H | D | D |
| 245 | CH₃ | CD₃ | H | H | H | D | D | H |
| 246 | CH₃ | CD₃ | H | H | H | D | H | H |
| 247 | CH₃ | CD₃ | H | H | H | H | D | H |
| 248 | CH₃ | CD₃ | H | H | H | D | D | D |
| 249 | CH₃ | CD₃ | H | H | H | D | H | D |
| 250 | CH₃ | CD₃ | H | H | H | H | D | D |
| 251 | CH₃ | CD₃ | H | H | D | D | D | H |
| 252 | CH₃ | CD₃ | H | H | D | D | H | H |
| 253 | CH₃ | CD₃ | H | H | D | H | D | H |
| 254 | CH₃ | CD₃ | H | H | D | D | D | D |
| 255 | CH₃ | CD₃ | H | H | D | D | H | D |
| 256 | CH₃ | CD₃ | H | H | D | H | D | D |
| 257 | CH₃ | CD₃ | H | D | H | D | D | H |
| 258 | CH₃ | CD₃ | H | D | H | D | H | H |
| 259 | CH₃ | CD₃ | H | D | H | H | D | H |
| 260 | CH₃ | CD₃ | H | D | H | D | D | D |
| 261 | CH₃ | CD₃ | H | D | H | D | H | D |
| 262 | CH₃ | CD₃ | H | D | H | H | D | D |
| 263 | CH₃ | CD₃ | H | D | D | D | D | H |
| 264 | CH₃ | CD₃ | H | D | D | D | H | H |
| 265 | CH₃ | CD₃ | H | D | D | H | D | H |
| 266 | CH₃ | CD₃ | H | D | D | D | D | D |
| 267 | CH₃ | CD₃ | H | D | D | D | H | D |
| 268 | CH₃ | CD₃ | H | D | D | H | D | D |
| 269 | CH₃ | CD₃ | D | H | H | D | D | H |
| 270 | CH₃ | CD₃ | D | H | H | D | H | H |
| 271 | CH₃ | CD₃ | D | H | H | H | D | H |
| 272 | CH₃ | CD₃ | D | H | H | D | D | D |
| 273 | CH₃ | CD₃ | D | H | H | D | H | D |
| 274 | CH₃ | CD₃ | D | H | H | H | D | D |
| 275 | CH₃ | CD₃ | D | H | D | D | D | H |
| 276 | CH₃ | CD₃ | D | H | D | D | H | H |
| 277 | CH₃ | CD₃ | D | H | D | H | D | H |
| 278 | CH₃ | CD₃ | D | H | D | D | D | D |
| 279 | CH₃ | CD₃ | D | H | D | D | H | D |
| 280 | CH₃ | CD₃ | D | H | D | H | D | D |
| 281 | CH₃ | CD₃ | D | D | H | D | D | H |
| 282 | CH₃ | CD₃ | D | D | H | D | H | H |
| 283 | CH₃ | CD₃ | D | D | H | H | D | H |
| 284 | CH₃ | CD₃ | D | D | H | D | D | D |
| 285 | CH₃ | CD₃ | D | D | H | D | H | D |
| 286 | CH₃ | CD₃ | D | D | H | H | D | D |
| 287 | CH₃ | CD₃ | D | D | D | D | D | H |
| 288 | CH₃ | CD₃ | D | D | D | D | H | H |
| 289 | CH₃ | CD₃ | D | D | D | H | D | H |
| 290 | CH₃ | CD₃ | D | D | D | D | D | D |
| 291 | CH₃ | CD₃ | D | D | D | D | H | D |
| 292 | CH₃ | CD₃ | D | D | D | H | D | D |
| 293 | CD₃ | CH₃ | H | H | H | D | D | H |
| 294 | CD₃ | CH₃ | H | H | H | D | H | H |
| 295 | CD₃ | CH₃ | H | H | H | H | D | H |
| 296 | CD₃ | CH₃ | H | H | H | D | D | D |
| 297 | CD₃ | CH₃ | H | H | H | D | H | D |
| 298 | CD₃ | CH₃ | H | H | H | H | D | D |
| 299 | CD₃ | CH₃ | H | H | D | D | D | H |
| 300 | CD₃ | CH₃ | H | H | D | D | H | H |
| 301 | CD₃ | CH₃ | H | H | D | H | D | H |
| 302 | CD₃ | CH₃ | H | H | D | D | D | D |
| 303 | CD₃ | CH₃ | H | H | D | D | H | D |
| 304 | CD₃ | CH₃ | H | H | D | H | D | D |
| 305 | CD₃ | CH₃ | H | D | H | D | D | H |
| 306 | CD₃ | CH₃ | H | D | H | D | H | H |
| 307 | CD₃ | CH₃ | H | D | H | H | D | H |
| 308 | CD₃ | CH₃ | H | D | H | D | D | D |
| 309 | CD₃ | CH₃ | H | D | H | D | H | D |
| 310 | CD₃ | CH₃ | H | D | H | H | D | D |
| 311 | CD₃ | CH₃ | H | D | D | D | D | H |
| 312 | CD₃ | CH₃ | H | D | D | D | H | H |
| 313 | CD₃ | CH₃ | H | D | D | H | D | H |
| 314 | CD₃ | CH₃ | H | D | D | D | D | D |
| 315 | CD₃ | CH₃ | H | D | D | D | H | D |
| 316 | CD₃ | CH₃ | H | D | D | H | D | D |
| 317 | CD₃ | CH₃ | D | H | H | D | D | H |
| 318 | CD₃ | CH₃ | D | H | H | D | H | H |
| 319 | CD₃ | CH₃ | D | H | H | H | D | H |
| 320 | CD₃ | CH₃ | D | H | H | D | D | D |
| 321 | CD₃ | CH₃ | D | H | H | D | H | D |
| 322 | CD₃ | CH₃ | D | H | H | H | D | D |
| 323 | CD₃ | CH₃ | D | H | D | D | D | H |
| 324 | CD₃ | CH₃ | D | H | D | D | H | H |
| 325 | CD₃ | CH₃ | D | H | D | H | D | H |
| 326 | CD₃ | CH₃ | D | H | D | D | D | D |
| 327 | CD₃ | CH₃ | D | H | D | D | H | D |
| 328 | CD₃ | CH₃ | D | H | D | H | D | D |
| 329 | CD₃ | CH₃ | D | D | H | D | D | H |
| 330 | CD₃ | CH₃ | D | D | H | D | H | H |
| 331 | CD₃ | CH₃ | D | D | H | H | D | H |
| 332 | CD₃ | CH₃ | D | D | H | D | D | D |
| 333 | CD₃ | CH₃ | D | D | H | D | H | D |
| 334 | CD₃ | CH₃ | D | D | H | H | D | D |
| 335 | CD₃ | CH₃ | D | D | D | D | D | H |
| 336 | CD₃ | CH₃ | D | D | D | D | H | H |
| 337 | CD₃ | CH₃ | D | D | D | H | D | H |
| 338 | CD₃ | CH₃ | D | D | D | D | D | D |
| 339 | CD₃ | CH₃ | D | D | D | D | H | D |
| 340 | CD₃ | CH₃ | D | D | D | H | D | D |
| 341 | CD₃ | CD₃ | H | H | H | D | D | H |
| 342 | CD₃ | CD₃ | H | H | H | D | H | H |
| 343 | CD₃ | CD₃ | H | H | H | H | D | H |
| 344 | CD₃ | CD₃ | H | H | H | D | D | D |
| 345 | CD₃ | CD₃ | H | H | H | D | H | D |
| 346 | CD₃ | CD₃ | H | H | H | H | D | D |
| 347 | CD₃ | CD₃ | H | H | D | D | D | H |
| 348 | CD₃ | CD₃ | H | H | D | D | H | H |
| 349 | CD₃ | CD₃ | H | H | D | H | D | H |
| 350 | CD₃ | CD₃ | H | H | D | D | D | D |
| 351 | CD₃ | CD₃ | H | H | D | D | H | D |
| 352 | CD₃ | CD₃ | H | H | D | H | D | D |
| 353 | CD₃ | CD₃ | H | D | H | D | D | H |
| 354 | CD₃ | CD₃ | H | D | H | D | H | H |
| 355 | CD₃ | CD₃ | H | D | H | H | D | H |
| 356 | CD₃ | CD₃ | H | D | H | D | D | D |
| 357 | CD₃ | CD₃ | H | D | H | D | H | D |
| 358 | CD₃ | CD₃ | H | D | H | H | D | D |
| 359 | CD₃ | CD₃ | H | D | D | D | D | H |
| 360 | CD₃ | CD₃ | H | D | D | D | H | H |
| 361 | CD₃ | CD₃ | H | D | D | H | D | H |
| 362 | CD₃ | CD₃ | H | D | D | D | D | D |
| 363 | CD₃ | CD₃ | H | D | D | D | H | D |
| 364 | CD₃ | CD₃ | H | D | D | H | D | D |
| 365 | CD₃ | CD₃ | D | H | H | D | D | H |
| 366 | CD₃ | CD₃ | D | H | H | D | H | H |
| 367 | CD₃ | CD₃ | D | H | H | H | D | H |
| 368 | CD₃ | CD₃ | D | H | H | D | D | D |
| 369 | CD₃ | CD₃ | D | H | H | D | H | D |
| 370 | CD₃ | CD₃ | D | H | H | H | D | D |
| 371 | CD₃ | CD₃ | D | H | D | D | D | H |
| 372 | CD₃ | CD₃ | D | H | D | D | H | H |
| 373 | CD₃ | CD₃ | D | H | D | H | D | H |
| 374 | CD₃ | CD₃ | D | H | D | D | D | D |
| 375 | CD₃ | CD₃ | D | H | D | D | H | D |
| 376 | CD₃ | CD₃ | D | H | D | H | D | D |
| 377 | CD₃ | CD₃ | D | D | H | D | D | H |
| 378 | CD₃ | CD₃ | D | D | H | D | H | H |
| 379 | CD₃ | CD₃ | D | D | H | H | D | H |
| 380 | CD₃ | CD₃ | D | D | H | D | D | D |
| 381 | CD₃ | CD₃ | D | D | H | D | H | D |
| 382 | CD₃ | CD₃ | D | D | H | H | D | D |
| 383 | CD₃ | CD₃ | D | D | D | D | D | H |
| 384 | CD₃ | CD₃ | D | D | D | D | H | H |
| 385 | CD₃ | CD₃ | D | D | D | H | D | H |
| 386 | CD₃ | CD₃ | D | D | D | D | D | D |
| 387 | CD₃ | CD₃ | D | D | D | D | H | D |
| 388 | CD₃ | CD₃ | D | D | D | H | D | D |

In some embodiments, the compound is selected from any one of the Compounds set forth in Table 3 (above), wherein any atom not designated as deuterium is present at its natural isotopic abundance.

In some embodiments, the compound of structural formula (I) or structural formula (II) is selected from any one of the Compounds set forth in Table 4 (below), wherein X is —OH; Y^{1a} and Y^{1b} are the same; Y^{2a} and Y^{2b} are the same; Y^{3a} and Y^{3b} are the same; Y^{4a} and Y^{4b} are the same; and Y⁷, Y⁸, Y⁹, Y¹⁰, and Y¹¹ are each hydrogen:

TABLE 4

| Compound # | R¹ | R² | Y^{1a}/Y^{1b} | Y^{2a}/Y^{2b} | Y^{3a}/Y^{3b} | Y^{4a}/Y^{4b} | Y⁵ | Y⁶ |
|---|---|---|---|---|---|---|---|---|
| 100 | CH₃ | CH₃ | H | H | H | H | H | D |
| 107 | CH₃ | CH₃ | D | H | H | H | H | H |
| 155 | CD₃ | CD₃ | D | H | H | H | H | H |
| 115 | CH₃ | CD₃ | H | H | H | H | H | H |
| 131 | CD₃ | ch₃ | H | H | H | H | H | H |
| 147 | CD₃ | CD₃ | H | H | H | H | H | H |
| 148 | CD₃ | CD₃ | H | H | H | H | H | D |
| 151 | CD₃ | CD₃ | H | D | H | H | H | H |
| 159 | CD₃ | CD₃ | D | D | H | H | H | H |
| 203 | CH₃ | CH₃ | H | H | D | D | D | H |
| 215 | CH₃ | CH₃ | H | D | D | D | D | H |
| 347 | CD₃ | CD₃ | H | H | D | D | D | H |
| 359 | CD₃ | CD₃ | H | D | D | D | D | H |
| 383 | CD₃ | CD₃ | D | D | D | D | D | H |

In some embodiments, the compound is selected from any one of the Compounds set forth in Table 4 (above), wherein any atom not designated as deuterium is present at its natural isotopic abundance.

In some embodiments of a compound of this invention, when Y^{1a} or Y^{1b} is deuterium, the level of deuterium incorporation at each Y^{1a} or Y^{1b} designated as deuterium is at least 52.5%, at least 75%, at least 82.5%, at least 90%, at least 95%, at least 97%, or at least 99%.

In some embodiments of a compound of this invention, when Y^{2a} or Y^{2b} is deuterium, the level of deuterium incorporation at each Y^{2a} or Y^{2b} designated as deuterium is at least 52.5%, at least 75%, at least 82.5%, at least 90%, at least 95%, at least 97%, or at least 99%.

In some embodiments of a compound of this invention, when Y^{3a} or Y^{3b} is deuterium, the level of deuterium incorporation at each Y^{3a} or Y^{3b} designated as deuterium is at least 52.5%, at least 75%, at least 82.5%, at least 90%, at least 95%, at least 97%, or at least 99%.

In some embodiments of a compound of this invention, when Y^{4a} or Y^{4b} is deuterium, the level of deuterium incorporation at each Y^{4a} or Y^{4b} designated as deuterium is at least 52.5%, at least 75%, at least 82.5%, at least 90%, at least 95%, at least 97%, or at least 99%.

In some embodiments of a compound of this invention, when Y⁵ is deuterium, the level of deuterium incorporation at each Y⁵ designated as deuterium is at least 52.5%, at least 75%, at least 82.5%, at least 90%, at least 95%, at least 97%, or at least 99%.

In some embodiments of a compound of this invention, when Y⁶ is deuterium, the level of deuterium incorporation at each Y⁶ designated as deuterium is at least 52.5%, at least 75%, at least 82.5%, at least 90%, at least 95%, at least 97%, or at least 99%.

In some embodiments of a compound of this invention, when Y⁷ is deuterium, the level of deuterium incorporation at each Y⁷ designated as deuterium is at least 52.5%, at least 75%, at least 82.5%, at least 90%, at least 95%, at least 97%, or at least 99%.

In some embodiments of a compound of this invention, when Y⁸ is deuterium, the level of deuterium incorporation at each Y⁸ designated as deuterium is at least 52.5%, at least 75%, at least 82.5%, at least 90%, at least 95%, at least 97%, or at least 99%.

In some embodiments of a compound of this invention, when Y⁹ is deuterium, the level of deuterium incorporation at each Y⁹ designated as deuterium is at least 52.5%, at least 75%, at least 82.5%, at least 90%, at least 95%, at least 97%, or at least 99%.

In some embodiments of a compound of this invention, when Y¹⁰ is deuterium, the level of deuterium incorporation at each Y¹⁰ designated as deuterium is at least 52.5%, at least 75%, at least 82.5%, at least 90%, at least 95%, at least 97%, or at least 99%.

In some embodiments of a compound of this invention, when Y¹¹ is deuterium, the level of deuterium incorporation at each Y¹¹ designated as deuterium is at least 52.5%, at least 75%, at least 82.5%, at least 90%, at least 95%, at least 97%, or at least 99%.

In some embodiments of a compound of this invention, when R¹ is —CH₂D, —CHD₂, or —CD₃, the level of deuterium incorporation at each designated deuterium of R¹ is at least 52.5%, at least 75%, at least 82.5%, at least 90%, at least 95%, at least 97%, or at least 99%.

In some embodiments of a compound of this invention, when R² is —CH₂D, —CHD₂, or —CD₃, the level of deuterium incorporation at each designated deuterium of R² is at least 52.5%, at least 75%, at least 82.5%, at least 90%, at least 95%, at least 97%, or at least 99%.

In another set of embodiments, any atom not designated as deuterium in any of the embodiments set forth herein is present at its natural isotopic abundance.

In some embodiments of a compound of this invention, deuterium incorporation at each designated deuterium atom is at least 52.5%, at least 75%, at least 82.5%, at least 90%, at least 95%, at least 97%, or at least 99%.

In some embodiments of a compound of this invention, at least one of Y^{1a}, Y^{1b}, Y^{2a}, Y^{2b}, Y^{3a}, Y^{3b}, Y^{4a}, Y^{4b}, Y⁵, Y⁶, Y⁷, Y⁸, Y⁹, Y¹⁰, and Y¹¹ is hydrogen, R¹ is —CH₃, —CH₂D, or —CHD₂, or R² is —CH₃, —CH₂D, or —CHD₂.

The synthesis of compounds of structural formula (I) can be readily achieved by synthetic chemists of ordinary skill by reference to the Exemplary Synthesis and Examples disclosed herein. Relevant procedures analogous to those of use for the preparation of compounds of structural formula (I) and intermediates thereof are disclosed, for instance in U.S. Patent Publication No 2005/0,261,341; Huck, L., et. al., Org. Lett., 2017, 19, 3747-3750; Reddy, B. P., et. al., WO 2017017630 A1; Winkler, M., et. al., Adv. Synth. Cat., 2007, 8+9, 1475-1480; Barker, O., et. al., WO 2012035023 A1; Ratton, S., et. al., EP0037353A1; Huet, L., et. al., Synlett, 2012, 23, 1230-1234; Senaweera, S., et. al., Chem. Comm., 2017, 53, 7545-7548; Shaik, S., et. al., Eur. J. Med. Chem., 2017, 126, 36-51; Mahtab, R., et. al., J. Chem. Pharm. Sci., 2014, 7, 34-38; Sakamuri, S., et. al., Bioorg. Med. Chem. Lett., 2001, 11, 495-500.

Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure.

### Exemplary Synthesis

A convenient method for synthesizing compounds of structural formula (I) is depicted in Scheme 1.

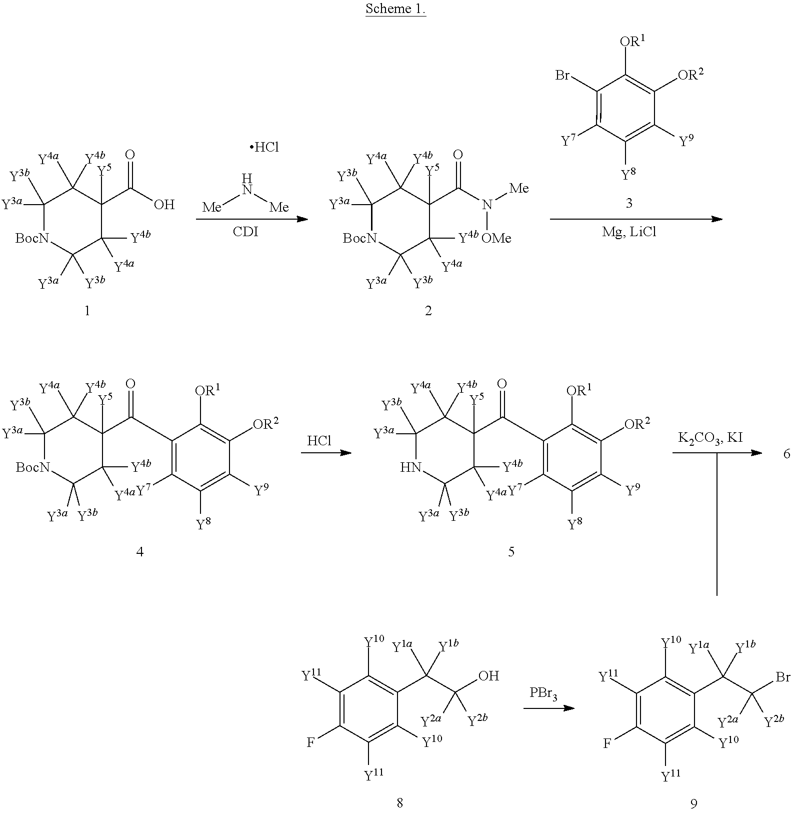

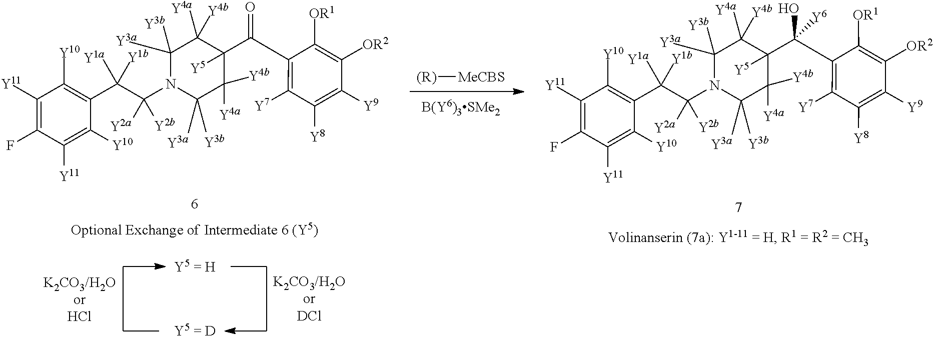

The synthesis of volinanserin (7a) has been described by Laux et. al. in U.S. Patent Publication No. 2005/0261341 and begins with conversion of carboxylic acid 1 to the corresponding Weinreb amide 2 via treatment with methoxymethylamine and CDI (carbonyldiimidazole). Reaction of 2 with the Grignard reagent generated from aryl bromide 3 then affords ketone 4. This is a modification of the route described by Laux et al. in order to allow for the preparation of volinanserin analogs with asymmetric deuteration patterns at positions R¹, R², Y⁷, Y⁸ and Y⁹. Shown in the following is an all-protio example depicting a reaction between the proposed aryl Grignard and a Weinreb amide as described in Huck, L., et. al., Org. Lett., 2017, 19, 3747-3750.

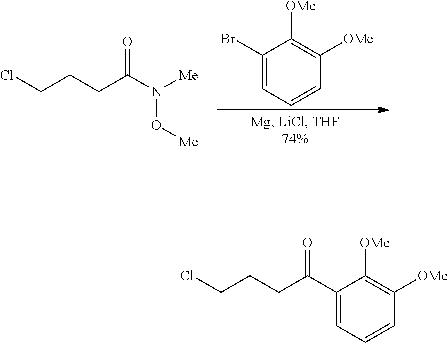 [SMILES: CON(C)C(=O)CCCCl ; COc1cccc(Br)c1OC ; COc1cccc(C(=O)CCCCl)c1OC]

Removal of the Boc protecting group followed by reaction with alkylbromide 9 (prepared in one step from primary alcohol 8) then generates ketone 6 which is readily converted to volinanserin via asymmetric reduction using borane-dimethylsulfide in the presence of a chiral catalyst ((R)-Methyl-CBS).

A final exchange process of intermediate 6 can be employed (using K₂CO₃/D₂ or DCl) to obtain high levels of % D at position Y⁵ prior to the final asymmetric reduction. Alternatively, subjecting intermediate 6 to K₂CO₃/H₂O or HCl can serve to fully de-enrich position Y⁵ if high levels of % H are required at this stage.

As shown above, accessing the deuterated analogs presented in Scheme 1 requires the following synthetic intermediates and reagents: 1, 3, 8 and BD₃·SMe₂. While the reagent BD₃·SMe₂ is commercially available from Cambridge Isotope Laboratories, access to deuterated analogs of intermediates 1, 3, and 8 requires individual synthetic preparation from commercially available deuterated precursors and reagents, as shown in Schemes 2-4 below.

The synthesis of the all-protio analog of carboxylic acid 1 (1a, Y^{3a}=Y^{3b}=Y^{4a}=Y^{4b}=Y⁵=H) is shown in Scheme 2a below. The first step in the sequence involves reduction of ketone 10a (commercially available from Sigma Aldrich) via treatment with sodium borohydride (NaBH₄) according to the procedure described by Reddy et. al. in WO 2017/017630A1. The resulting alcohol 11a is then converted to nitrile 12a via a two step procedure reported in Winkler et. al., Adv. Synth. Cat., 2007, 8+9,1475-1480. Nitrile 12a is then hydrolyzed to carboxylic acid 1a via treatment with aqueous potassium hydroxide following the procedure described by Barker et. al. in WO 2012/035023A1 for the hydrolysis of a structurally similar compound. Replacing sodium borohydride in Scheme 2a with sodium borodeuteride (NaBD₄ (99% D) commercially available from Cambridge Isotope Laboratories) then provides 1b (Y^{3a}=Y^{3b}=Y^{4a}=Y^{4b}=H, Y⁵=D) (Scheme 2b).

The remaining analogs (1c-1h) can be prepared in a similar fashion starting from the appropriately labelled commercially available starting material (10b (99% D) available from CDN Isotopes, 10c (95-98% D) available from APIChemical and 10d (98% D) available from CombiPhos) as depicted in Schemes 2c-2h below.

Use of appropriately deuterated reagents allows deuterium incorporation at the Y^{3a}, Y^{3b}, Y^{4a}, Y^{4b}, and Y⁵ positions of a compound of structural formula (I) (e.g., compound 7) or any appropriate intermediate herein, e.g., about 90%, about 95%, about 97%, about 98% or about 99% deuterium incorporation at any of Y^{3a}, Y^{3b}, Y^{4a}, Y^{4b} and Y⁵.

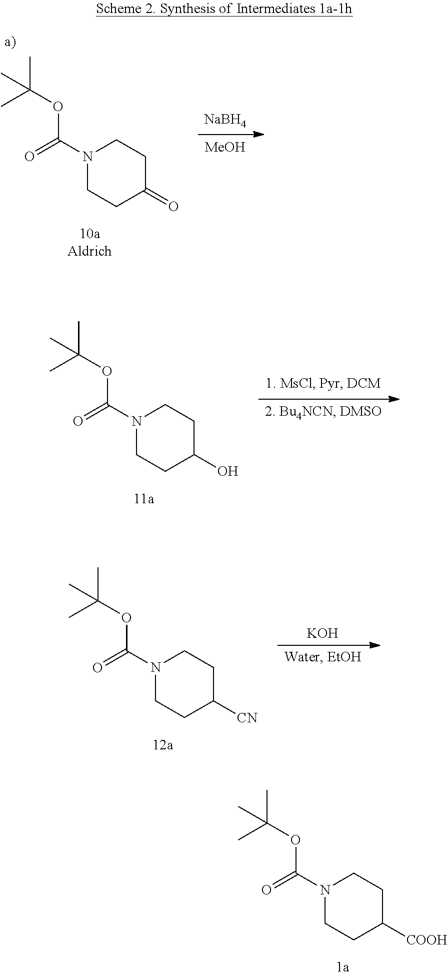

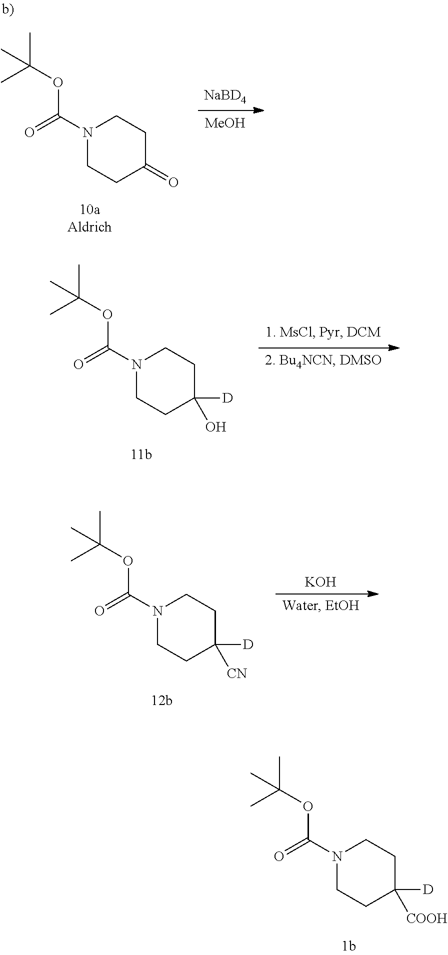

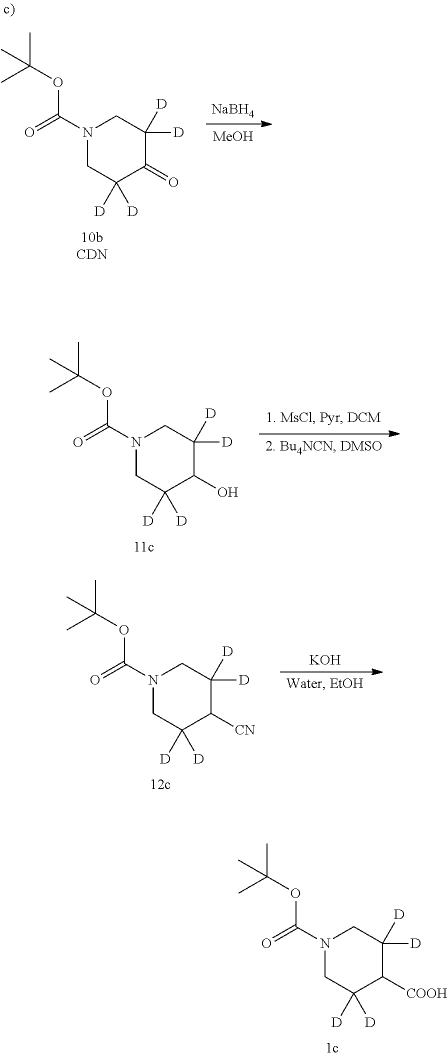

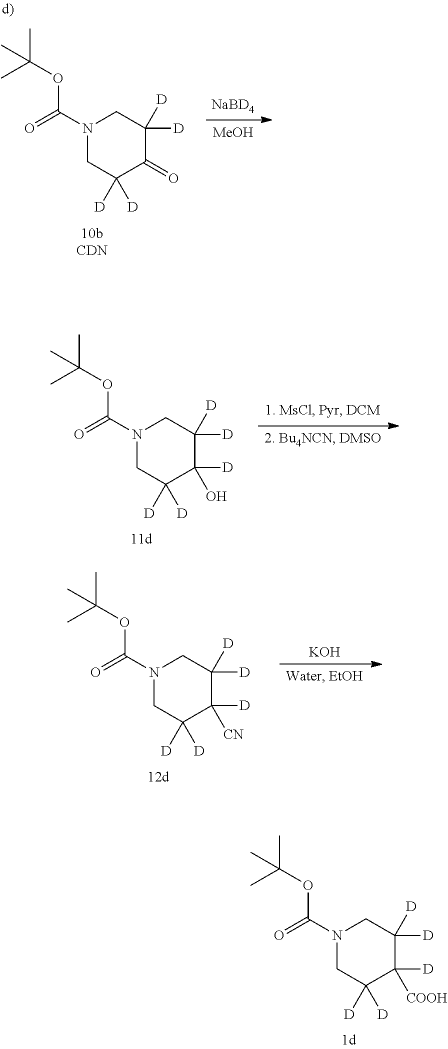

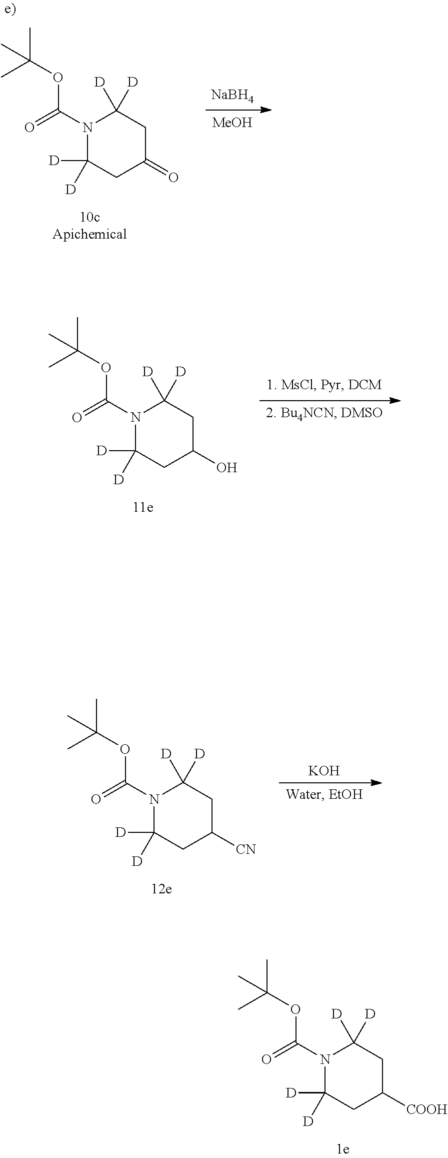

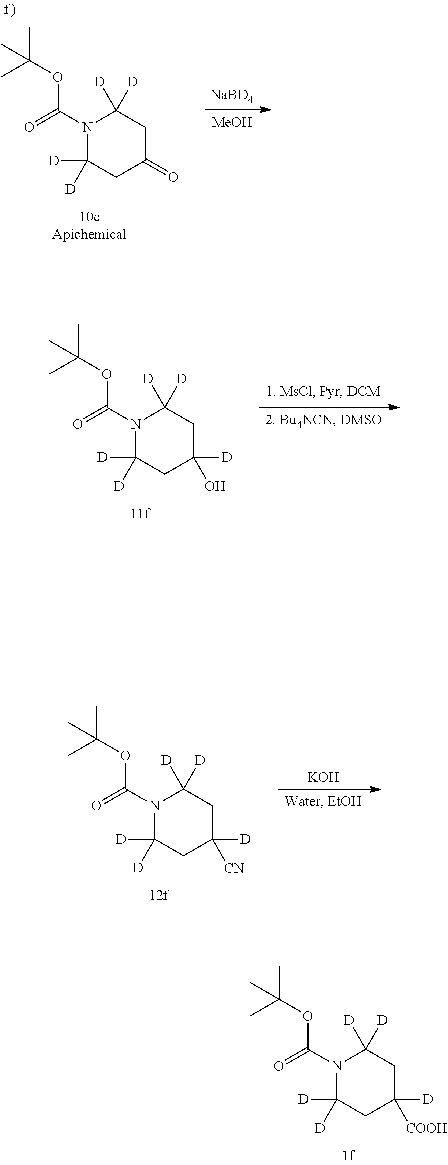

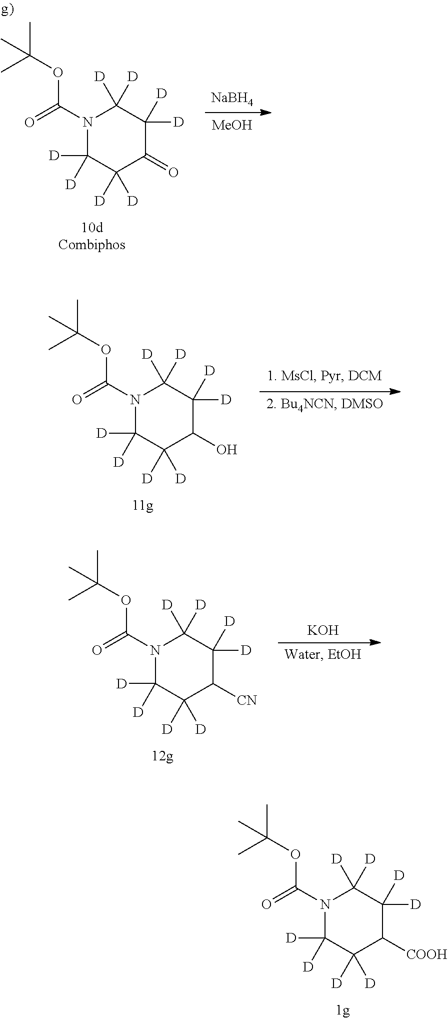

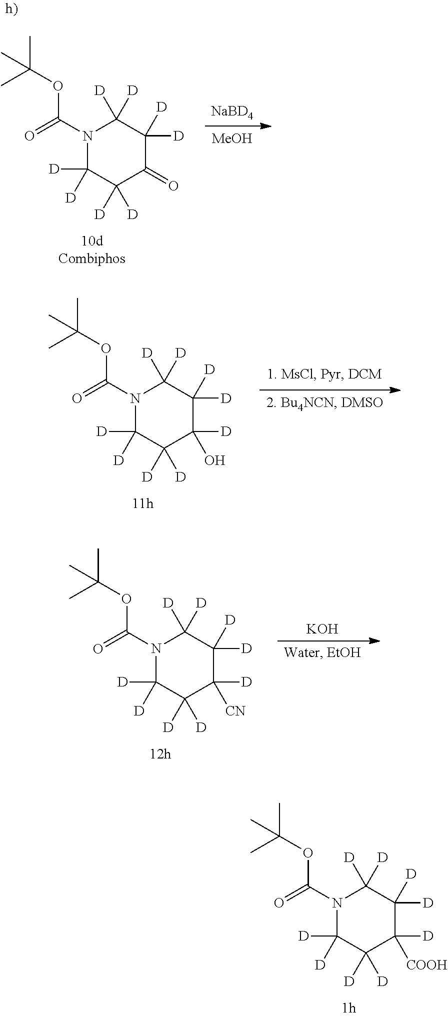

While the all-protio analog of aryl bromide 3 (3a, Y⁷=Y⁸=Y⁹=H; R¹=R²═CH₃) is commercially available from Alfa Aesar, its preparation following literature procedures is shown in Scheme 3a. The first step in the sequence involves reaction of catechol (13a, commercially available from Alfa Aesar) with methanol in the presence of sodium acetate and acetic acid to form monomethyl ether 14a as described by Ratton, S. et. al. in European Patent No. 0037353A1. As illustrated in Huet, L. et. al., Synlett, 2012, 23, 1230-1234, the resulting phenol may then be converted to 3a via a two-step process involving bromination followed by alkylation with methyl iodide.

The remaining analogs (3b-3h) can be prepared following the general route shown in Scheme 3a starting from either catechol (13a) or d₄-catechol (13b (96% D), commercially available from Aldrich) substituting d₄-methanol (99.8% D, commercially available from Aldrich) for methanol and/or d₃-methyl iodide (99.5% D, commercially available from Aldrich) for methyl iodide as appropriate (Schemes 3b-3h).

Use of appropriately deuterated reagents allows deuterium incorporation at the Y⁷, Y⁸, Y⁹, R¹, and R² positions of a compound of structural formula (I) (e.g., compound 7) or any appropriate intermediate herein, e.g., about 90%, about 95%, about 97%, about 98% or about 99% deuterium incorporation at any of Y⁷, Y⁸, Y⁹, R¹, and R².

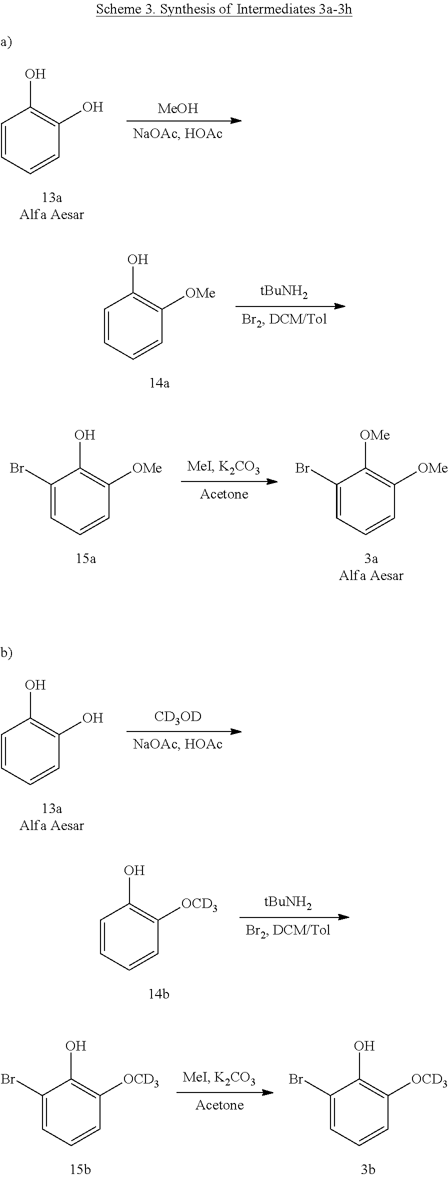

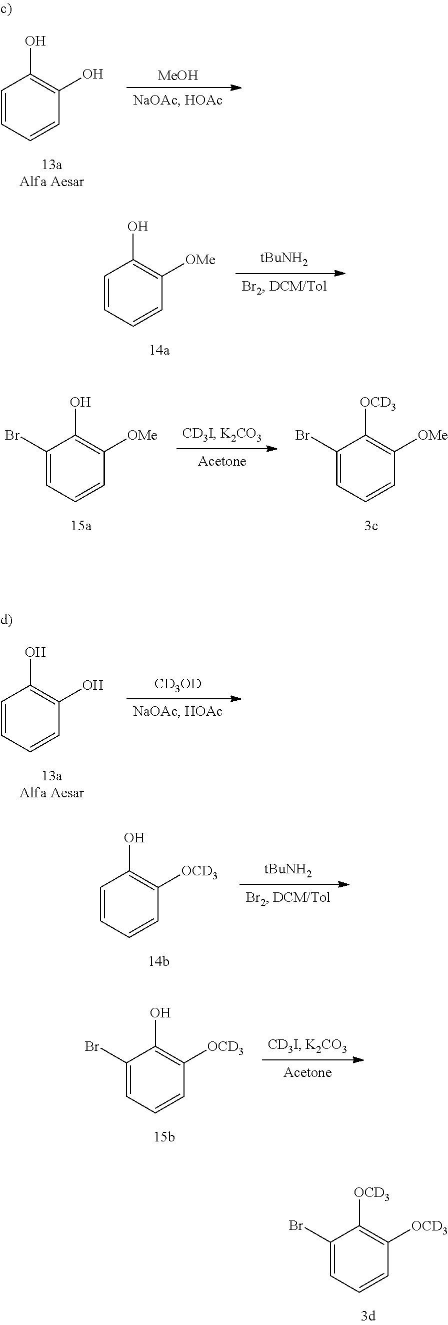

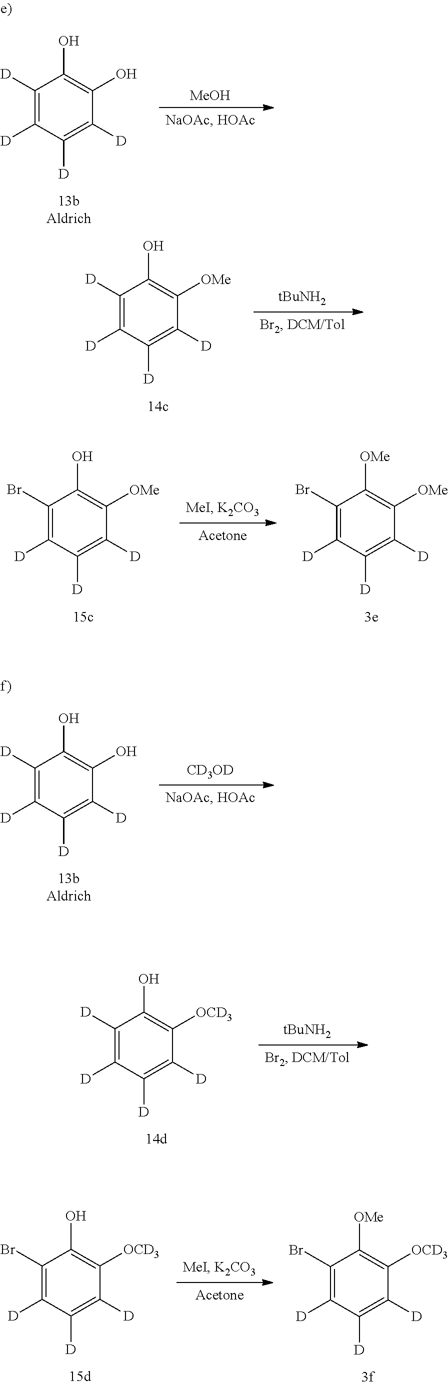

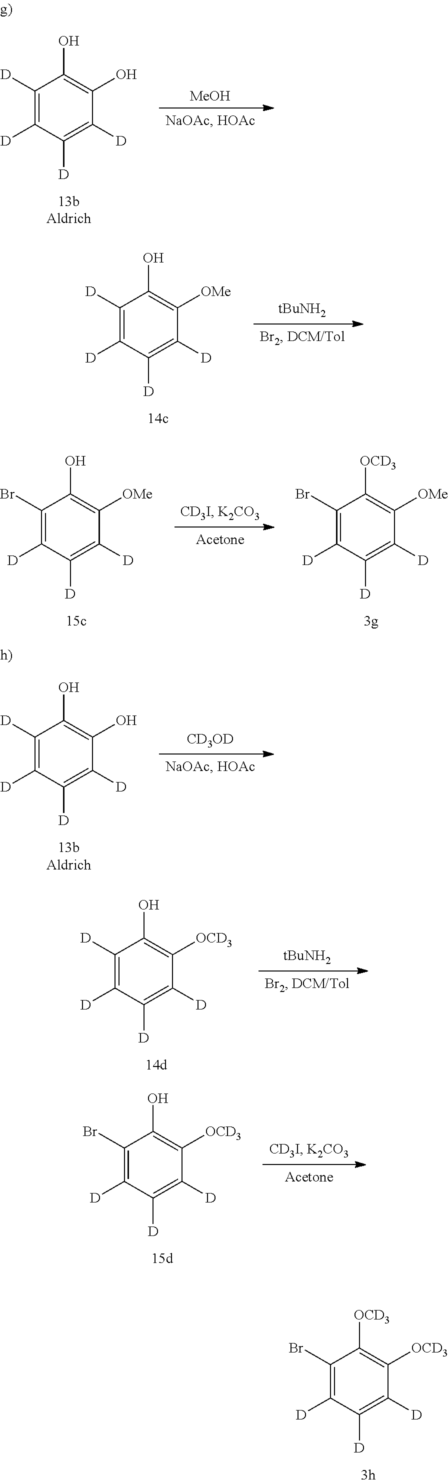

While the all-protio analog of primary alcohol 8 (8a, Y^{1a}=Y^{1b}=Y^{2a}=Y^{2b}=Y¹⁰=Y¹¹=H) is commercially available from Aldrich, its preparation following literature procedures is shown in Scheme 4a. The first step in the sequence involves reduction of 4-fluoro benzoic acid (16a, commercially available from Aldrich) with lithium aluminum hydride to afford benzyl alcohol 17a following the method described in Senaweera et. al., Chem. Comm., 2017, 53, 7545-7548. Subsequent treatment with phosphorous tribromide, as described in Shaik et. al., Eur. J Med. Chem., 2017, 126, 36-51, then provides alkyl bromide 18a which is then converted to carboxylic acid 19a via a two step procedure reported by Mahtab et. al., J. Chem. Pharm. Sci., 2014, 7, 34-38. Finally, primary alcohol 8a is obtained via reduction of carboxylic acid 19a with borane according to the procedure of Sakamuri et. al., Bioorg. Med. Chem. Lett., 2001, 11, 495-500.

The remaining analogs (8a-8h) can be prepared following the general route shown in Scheme 4a starting from either 4-fluorobenzoic acid (16a) or d₄-4-fluorobenzoic acid (16b (99% D), commercially available from CDN Isotopes) substituting LiAlD₄ (98% D, commercially available from Cambridge Isotope Laboratories) for LiAlH₄ and/or BD₃ (98% D, commercially available from Cambridge Isotope Laboratories) for BH₃ as appropriate (Schemes 8a-8h).

Use of appropriately deuterated reagents allows deuterium incorporation at the Y^{1a}, Y^{1b}, Y^{2a}, Y^{2b}, Y¹⁰, and Y¹¹ positions of a compound of Formula I (e.g., compound 7) or any appropriate intermediate herein, e.g., about 90%, about 95%, about 97%, about 98% or about 99% deuterium incorporation at any of Y^{1a}, Y^{1b}, Y^{2a}, Y^{2b}, Y¹⁰, and Y¹¹.

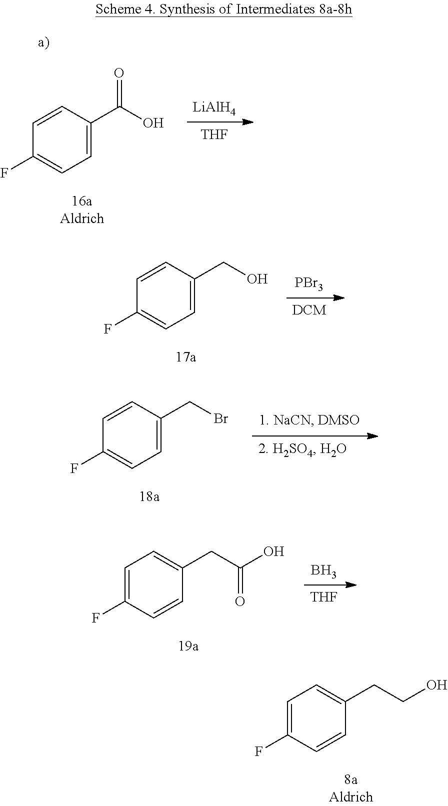

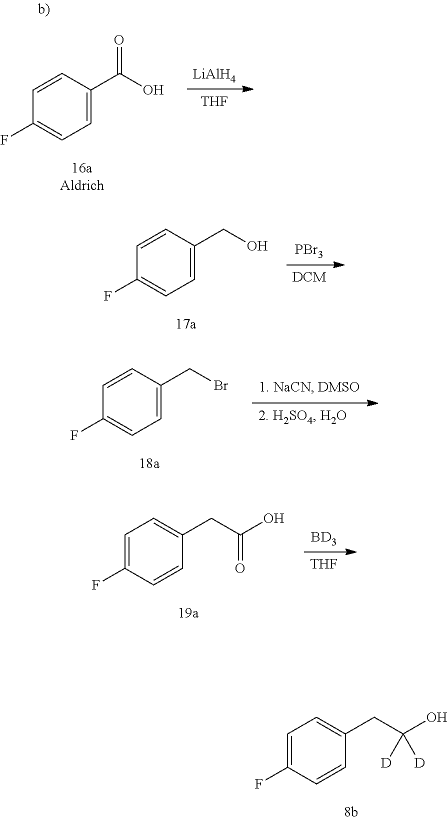

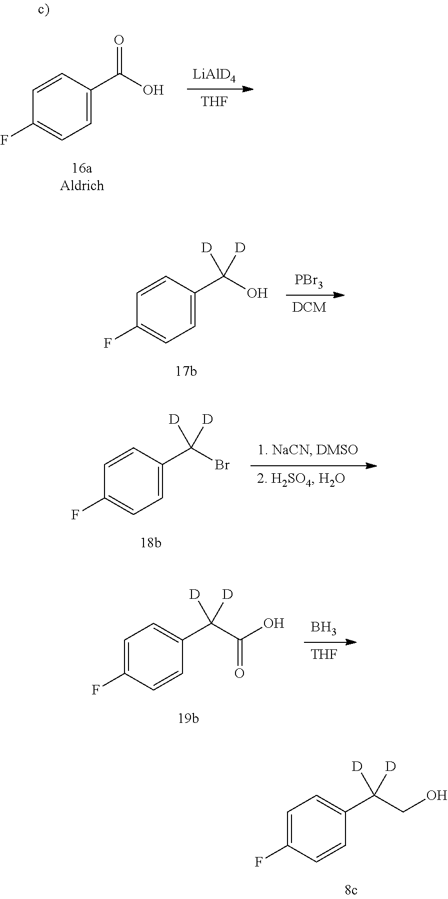

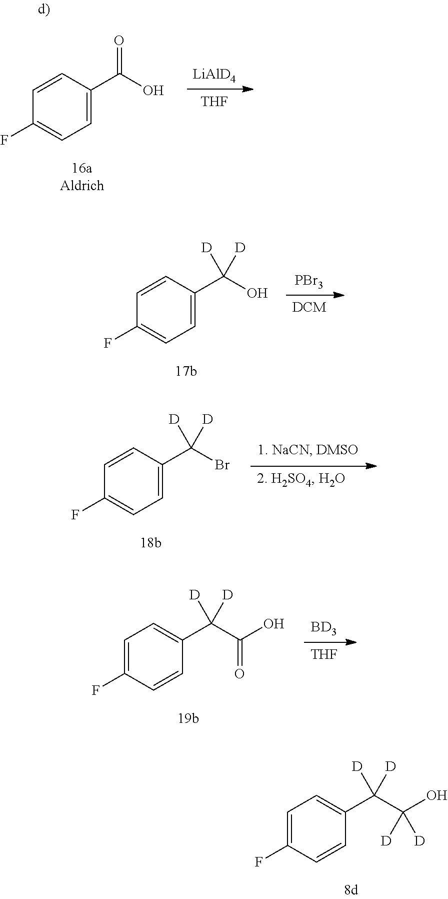

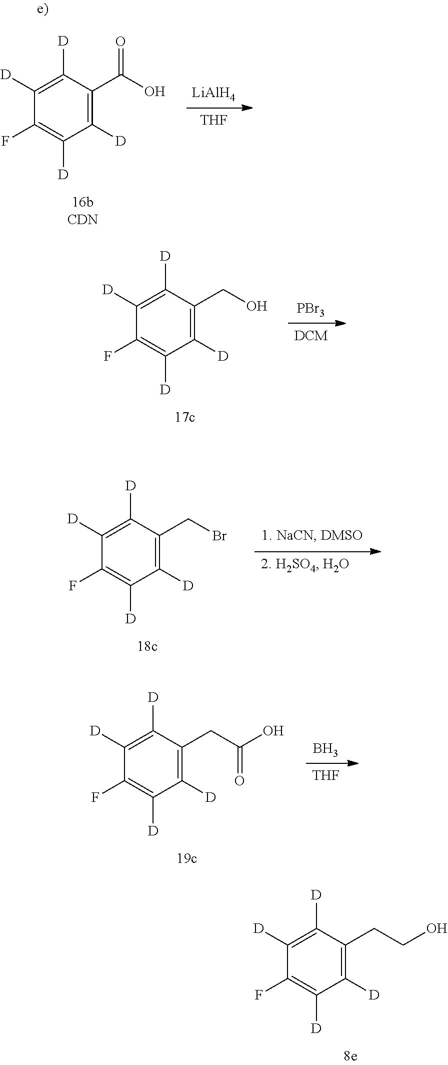

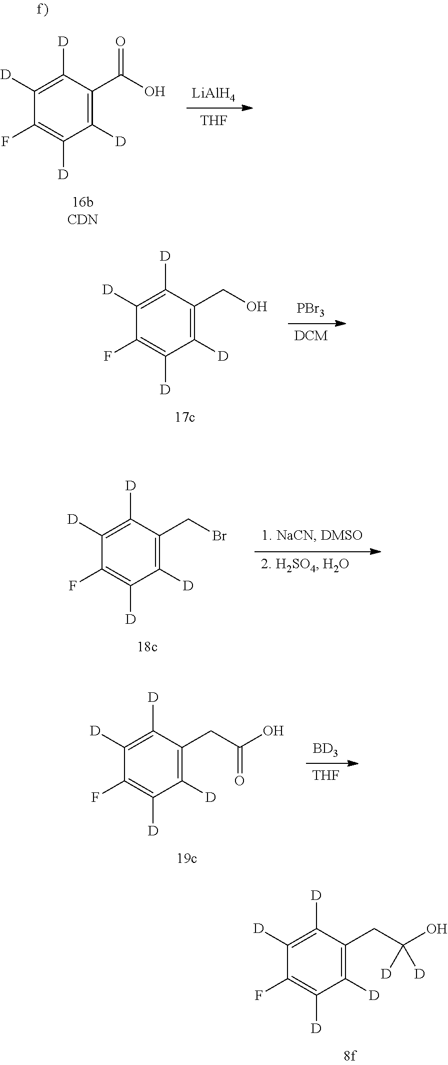

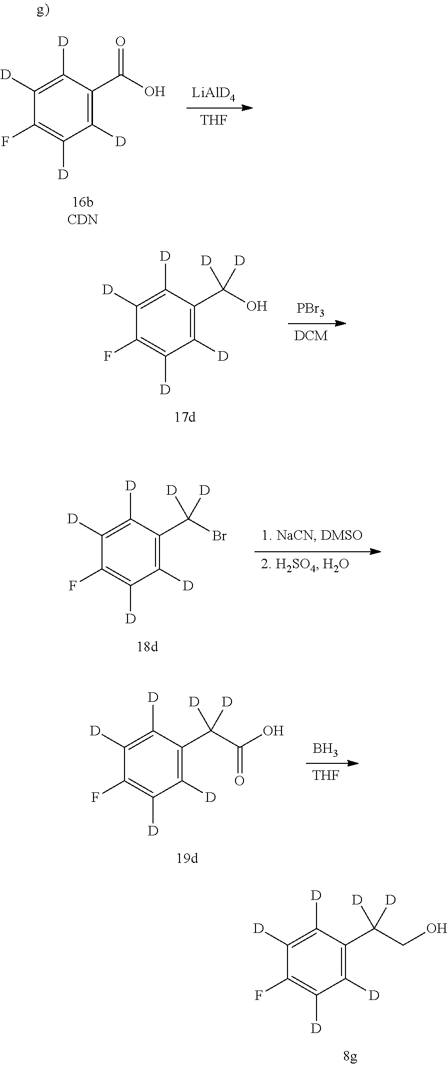

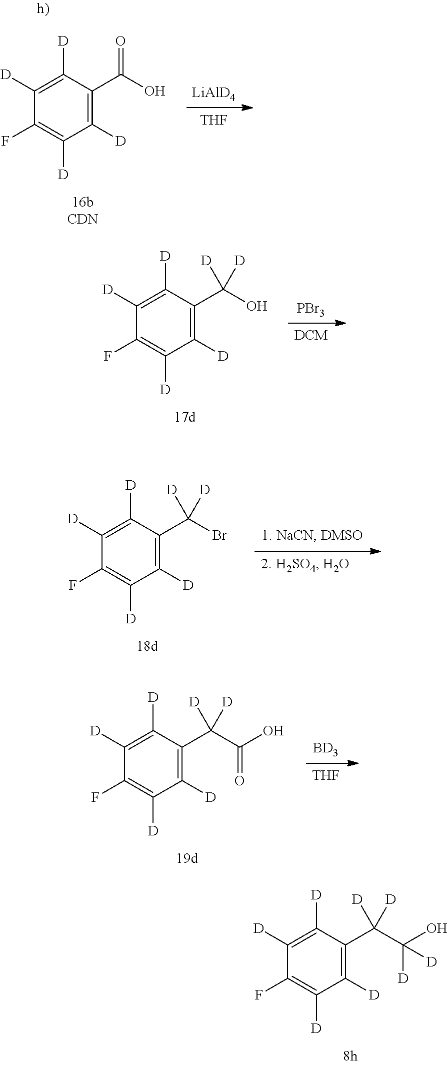

The proposed synthesis of d₇-Volinanserin (7h) is depicted in Scheme 5 below as a representative example using the deuterated intermediates described herein.

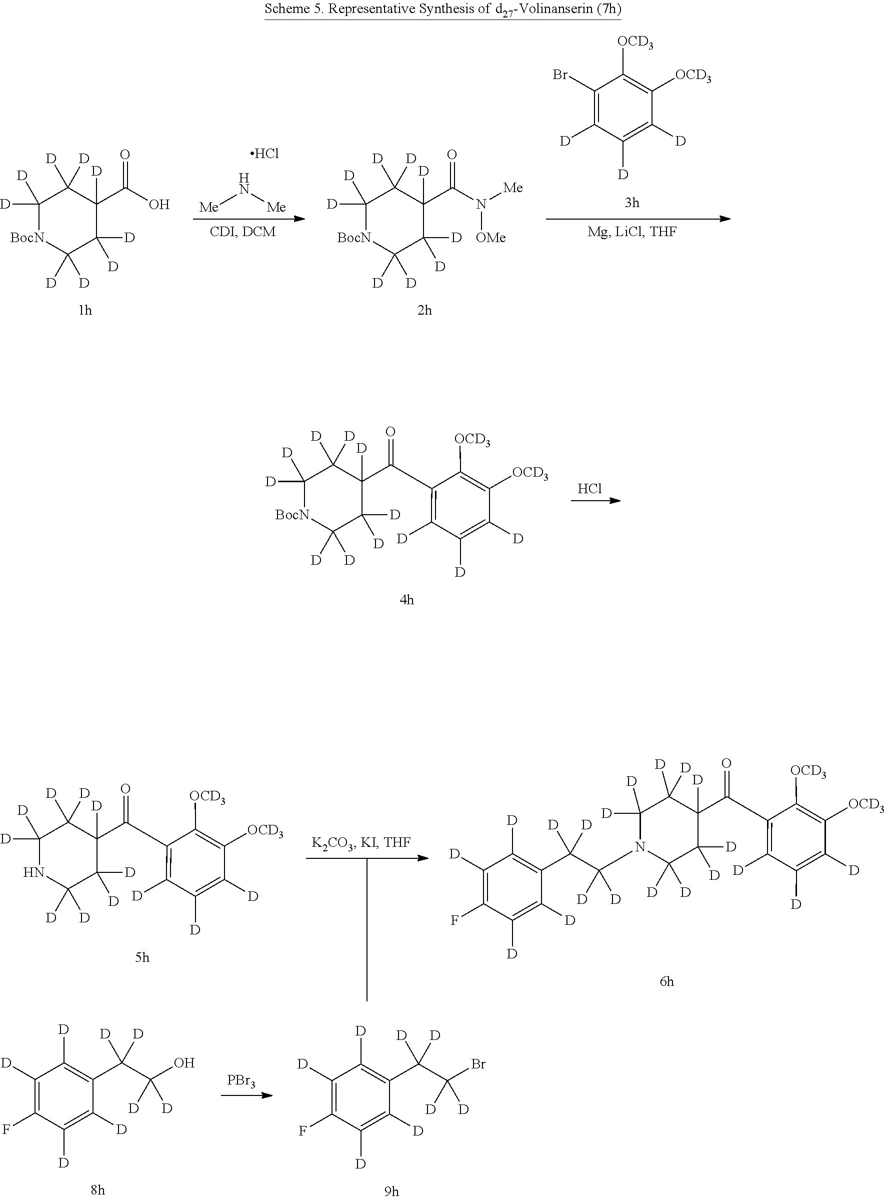

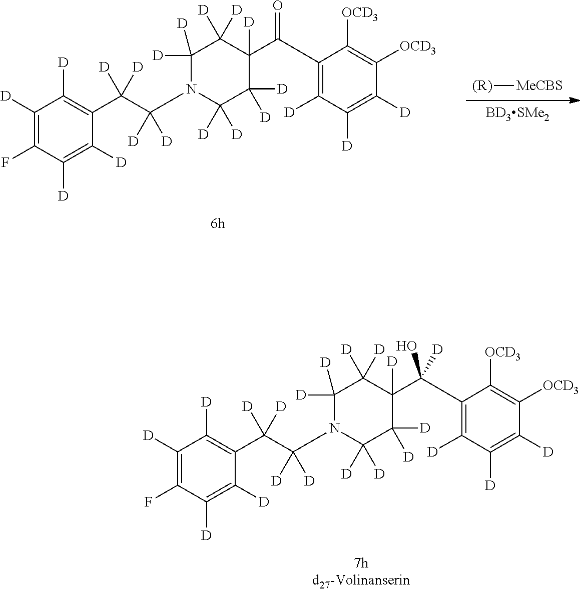

Use of appropriately deuterated reagents allows deuterium incorporation at the Y^{1a}, Y^{1b}, Y^{2a}, Y^{2b}, Y^{3a}, Y^{3b}, Y⁴a Y^{4b}, Y⁵, Y⁶, Y⁷, Y⁵, Y⁹, Y¹⁰, Y¹¹, R¹, and R² positions of a compound of structural formula (I) or any appropriate intermediate herein, e.g., about 90%, about 95%, about 97%, or about 99% deuterium incorporation at any Y^{1a}, Y^{1b}, Y^{2a}, Y^{2b}, Y^{3a}, Y^{3b}, Y^{4a}, Y^{4b}, Y⁵, Y⁶, Y⁷, Y⁸, Y⁹, Y¹⁰, Y¹¹, R¹, and/or R².

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², R³, etc.) or not. The suitability of a chemical group in a compound structure for use in the synthesis of another compound is within the knowledge of one of ordinary skill in the art.

Additional methods of synthesizing compounds of structural formula (I) and their synthetic precursors, including those within routes not explicitly shown in schemes herein, are within the means of chemists of ordinary skill in the art. The methods described herein can also additionally include steps, either before or after the steps described specifically herein, to add or remove suitable protecting groups in order to ultimately allow synthesis of the compounds herein. In addition, various synthetic steps can be performed in an alternate sequence or order to give the desired compounds. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene, T W, et al., Protective Groups in Organic Synthesis, 3^{rd} Ed., John Wiley and Sons (1999); Fieser, L., et al., Fieser and Fieser's Reagents for Organic Synthesis, John Wiley and Sons (1994); and Paquette, L, ed., Encyclopedia of Reagents for Organic Synthesis, John Wiley and Sons (1995) and subsequent editions thereof.

Combinations of substituents and variables envisioned by this invention are only those that result in the formation of stable compounds.

### Compositions

The invention also provides pharmaceutical compositions comprising an effective amount of a compound of structural formula (I) (e.g., of the first or second embodiment, or any embodiment or aspect of embodiment thereof described in the foregoing) or of structural formula (II), or a pharmaceutically acceptable salt of said compound; and a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, MD (20th ed. 2000).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

Another embodiment is a controlled release pharmaceutical composition comprising a compound of structural formula (I) (e.g., of any embodiment or aspect of embodiment described herein).

In one aspect of the controlled release pharmaceutical compositions, the controlled release pharmaceutical composition further comprises release controlling agent(s) and optionally pharmaceutically acceptable excipients.

The release controlling agents can be selected from hydrophilic release controlling agents, hydrophobic release controlling agents, or mixtures thereof.

The hydrophilic release controlling agents are selected from, but are not limited to, hydroxypropyl methyl cellulose (IPMC), hydroxypropyl cellulose (IPC), hydroxy ethyl cellulose (HEC), polyethylene oxide, polyvinyl alcohol, polyvinylpyrrolidone, xanthan gum, guar gum, chitosan and its derivatives, carbomer, carrageenan, carboxymethyl cellulose, sodium alginate, polyglycolized glycerides, polyethyleneglycol, or a mixture thereof.

The hydrophobic release controlling agents are selected from, but are not limited to, polyvinyl acetate dispersion, ethyl cellulose, cellulose acetate, cellulose propionate (lower, medium or higher molecular weight), cellulose acetate propionate, cellulose acetate butyrate, cellulose acetate phthalate, cellulosetriacetate, poly (methyl methacrylate), poly (ethyl methacrylate), poly (butyl methacrylate), poly (isobutyl methacrylate), and poly (hexyl methacrylate), poly (isodecyl methacrylate), poly (lauryl methacrylate), poly (phenyl methacrylate), poly (methyl acrylate), poly (isopropyl acrylate), poly (isobutyl acrylate), poly (octadecyl acrylate), waxes such as beeswax, camauba wax, paraffin wax, microcrystalline wax, and ozokerite; fatty alcohols such as cetostearyl alcohol, stearyl alcohol, cetyl alcohol andmyristyl alcohol, and fatty acid esters such as glyceryl mono stearate; glycerol monooleate, acetylated monoglycerides, tristearin, tripalmitin, cetyl esters wax, glyceryl palmitostearate, glyceryl behenate, or hydrogenated vegetable oils.

The amount of the release controlling agent can range from about 5% to about 95% by weight of the composition, more typically, from about 25% to about 75% by weight of the composition and, more preferably, from about 35% to about 65% by weight of the composition.

The pharmaceutically acceptable excipients include, but are not limited to, diluents, binders, solubilizing agents, dissolution enhancing agents, pore forming agents, osmagents, gas forming agents, lubricants and glidants known to persons skilled in the art.

Another embodiment is a controlled release pharmaceutical composition comprising a compound of structural formula (I) (or any embodiment or aspect of embodiment of the compound of structural formula (I)), a release controlling agent selected from hydrophilic release controlling agent, hydrophobic release controlling agent, and mixtures thereof, and optionally a pharmaceutically acceptable excipient.

Another embodiment is a controlled release pharmaceutical composition comprising a compound of structural formula (I) (or any embodiment or aspect of embodiment of the compound of structural formula (I)), a release controlling agent selected from hydrophilic release controlling agent, hydrophobic release controlling agent, and mixtures thereof, and optionally a pharmaceutically acceptable excipient, wherein the hydrophilic release controlling agent is selected from hydroxypropyl methyl cellulose (HPMC), hydroxypropyl cellulose (HPC), hydroxy ethyl cellulose (HEC), polyethylene oxide, polyvinyl alcohol, polyvinylpyrrolidone, xanthan gum, guar gum, chitosan and its derivatives, carbomer, carrageenan, carboxymethyl cellulose, sodium alginate, polyglycolized glycerides, polyethyleneglycol, and a mixture thereof.

Another embodiment is a controlled release pharmaceutical composition comprising a compound of structural formula (I) (or any embodiment or aspect of embodiment of the compound of structural formula (I)), a release controlling agent selected from hydrophilic release controlling agent, hydrophobic release controlling agent, and mixtures thereof, and optionally a pharmaceutically acceptable excipient, wherein the hydrophobic release controlling agent is selected from polyvinyl acetate dispersion, ethyl cellulose, cellulose acetate, cellulose propionate (lower, medium or higher molecular weight), cellulose acetate propionate, cellulose acetate butyrate, cellulose acetate phthalate, cellulosetriacetate, poly (methyl methacrylate), poly (ethyl methacrylate), poly (butyl methacrylate), poly (isobutyl methacrylate), and poly (hexyl methacrylate), poly (isodecyl methacrylate), poly (lauryl methacrylate), poly (phenyl methacrylate), poly (methyl acrylate), poly (isopropyl acrylate), poly (isobutyl acrylate), poly (octadecyl acrylate), waxes such as beeswax, camauba wax, paraffin wax, microcrystalline wax, and ozokerite; fatty alcohols such as cetostearyl alcohol, stearyl alcohol, cetyl alcohol andmyristyl alcohol, and fatty acid esters such as glyceryl mono stearate; glycerol monooleate, acetylated monoglycerides, tristearin, tripalmitin, cetyl esters wax, glyceryl palmitostearate, glyceryl behenate, and hydrogenated vegetable oils.

U.S. Patent Application Publication No. 2013/0143897, published Jun. 6, 2013, describes controlled release pharmaceutical compositions comprising blonanserin. In the described compositions, blonanserin can be substituted with compounds of structural formula (I) (or any embodiment or aspect of embodiment of the compound of structural formula (I)) to form controlled release pharmaceutical compositions of compounds of the present invention.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example, water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For topical application to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the subject, such organ or tissue may be bathed in a medium containing a composition of this invention, a composition of this invention may be painted onto the organ, or a composition of this invention may be applied in any other convenient way.

In another embodiment, a composition of this invention further comprises one or more additional therapeutic agents. The additional therapeutic agent may be selected from any compound or therapeutic agent known to have or that demonstrates advantageous properties when administered with a compound having the same mechanism of action as volinanserin. Such agents include those indicated as being useful in combination with volinanserin, including but not limited to, escitalopram.

Preferably, the additional therapeutic agent is an agent useful in the treatment of a disease or condition selected from psychosis, schizophrenia (including chronic schizoprenia), schizoaffective disorder, Parkinson's disease (including Parkinson's disease psychosis), Lewy body dementia, sleep disorder (including insomnia), agitation, mood disorder (including depression), thromboembolic disorder, autism, and attention deficit hyperactivity disorder.

In one embodiment, the additional therapeutic agent is escitalopram.

In another embodiment, the invention provides separate dosage forms of a compound of this invention and one or more of any of the above-described additional therapeutic agents, wherein the compound and additional therapeutic agent are associated with one another. The term “associated with one another” as used herein means that the separate dosage forms are packaged together or otherwise attached to one another such that it is readily apparent that the separate dosage forms are intended to be sold and administered together (e.g., within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to treat the target disease.

The term “subject in need thereof,” refers to a subject having or being diagnosed with a disease or condition selected from psychosis, schizophrenia (including chronic schizophrenia), schizoaffective disorder, Parkinson's disease (including Parkinson's disease psychosis), Lewy body dementia, sleep disorder (including insomnia), agitation, mood disorder (including depression), thromboembolic disorder, autism, and attention deficit hyperactivity disorder, or at risk for sustaining or developing such a disease or disorder.

The interrelationship of dosages for animals and humans (based on milligrams per meter squared of body surface) is described in Freireich et al., Cancer Chemother. Rep, 1966, 50: 219. Body surface area may be approximately determined from height and weight of the subject. See, e.g., Scientific Tables, Geigy Pharmaceuticals, Ardsley, N.Y., 1970, 537.

In one embodiment, an effective amount of a compound of this invention can range from 0.4 mg to 4 mg, from 0.2 mg to 10 mg, or from 0.02 mg to 20 mg. In a preferred embodiment the effective amount is 2 mg.

In one embodiment, an effective amount of a compound of this invention can range from 0.4 mg/day to 4 mg/day, from 0.2 mg/day to 10 mg/day, or from 0.02 mg/day to 20 mg/day. In a preferred embodiment the effective amount is 2 mg/day.

In one embodiment, an effective amount of a compound of this invention can range from 0.008 mg/kg to 0.08 mg/kg, from 0.004 mg/kg to 0.2 mg/kg, from 0.0004 mg/kg to 0.4 mg/kg. In a preferred embodiment the effective amount is 0.04 mg/kg.

In one embodiment, an effective amount of a compound of this invention can range from 0.008 mg/kg per day to 0.08 mg/kg per day, from 0.004 mg/kg per day to 0.2 mg/kg per day, from 0.0004 mg/kg per day to 0.4 mg/kg per day. In a preferred embodiment the effective amount is 0.04 mg/kg per day.

The effective amount can be administered once or twice daily, every other day, weekly or biweekly. In preferred embodiments, the effective amount is administered once daily. Effective doses will also vary, as recognized by those skilled in the art, depending on the diseases treated, the severity of the disease, the route of administration, the sex, age and general health condition of the subject, excipient usage, the possibility of co-usage with other therapeutic treatments such as use of other agents and the judgment of the treating physician. For example, guidance for selecting an effective dose can be determined by reference to the prescribing information for volinanserin.

For pharmaceutical compositions that comprise one or more additional therapeutic agents, an effective amount of the additional therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these additional therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references is incorporated herein by reference in itsentirety.

Some of the additional therapeutic agents referenced above may act synergistically with the compounds of this invention. When this occurs, it will allow the effective dosage of the additional therapeutic agent and/or the compound of this invention to be reduced from that required in a monotherapy. This has the advantage of minimizing toxic side effects of either the additional therapeutic agent of a compound of this invention, synergistically improving efficacy, improving ease of administration or use and/or reduced overall expense of compound preparation or formulation.

### Methods of Treatment

In other embodiments, the invention provides a method of antagonizing or inverse agonizing the activity of seretonin 5-HT_{2A} receptor in a cell, comprising contacting a cell with one or more compounds of structural formula (I) (e.g., of any embodiment or aspect of embodiment thereof) or structural formula (II), or a pharmaceutically acceptable salt thereof. In some embodiments, the cell is contacted in vitro. In some embodiments, the cell is contacted in vivo. In some embodiments, the cell is contacted ex vivo.

In certain embodiments, the invention provides a method of treating a disease that is beneficially treated by a compound of structural formula (I) in a subject in need thereof, comprising the step of administering to the subject an effective amount of a compound, or a pharmaceutically acceptable salt thereof, or a composition of this invention (including the pharmaceutical compositions and controlled release pharmaceutical compositions described herein). In certain embodiments, the subject is a patient in need of such treatment. In certain embodiments, the subject is a human.

In further embodiments, the invention provides a pharmaceutical composition for treating or preventing a disease or condition selected from psychosis, chronic schizophrenia (including chronic schizophrenia), schizoaffective disorder, Parkinson's disease (including Parkinson's disease psychosis), Lewy body dementia, sleep disorder (including insomnia), agitation, mood disorder (including depression), thromboembolic disorder, autism, attention deficit hyperactivity disorder, and any combination thereof, comprising a compound of structural formula (I) (e.g., of an embodiment or aspect of embodiment thereof described herein), or structural formula (II), or pharmaceutically acceptable salt thereof.

In further embodiments, the invention provides a compound of structural formula (I) (e.g., of an embodiment or aspect of embodiment thereof described herein) or structural formula (II), or pharmaceutically acceptable salt thereof, for use in treating or preventing a disease or condition selected from psychosis, schizophrenia (including chronic schizophrenia), schizoaffective disorder, Parkinson's disease (including Parkinson's disease psychosis), Lewy body dementia, sleep disorder (including insomnia), agitation, mood disorder (including depression), thromboembolic disorder, autism, attention deficit hyperactivity disorder, and any combination thereof.

In further embodiments, the invention provides the use of a compound of structural formula (I) (e.g., of an embodiment or aspect of embodiment thereof described herein) or structural formula (II), or pharmaceutically acceptable salt thereof, for the manufacture of a medicament for treating or preventing a disease or condition selected from psychosis, schizophrenia (including chronic schizophrenia), schizoaffective disorder, Parkinson's disease (including Parkinson's disease psychosis), Lewy body dementia, sleep disorder (including insomnia), agitation, mood disorder (including depression), thromboembolic disorder, autism, attention deficit hyperactivity disorder, and any combination thereof.

Diseases amenable to the methods of treatment disclosed herein are well known in the art and include, but are not limited to psychosis, schizophrenia (including chronic schizophrenia), schizoaffective disorder, Parkinson's disease (including Parkinson's disease psychosis), Lewy body dementia, sleep disorder (including insomnia), agitation, mood disorder (including depression), thromboembolic disorder, autism, and attention deficit hyperactivity disorder.

Identifying a subject in need of such treatment can be in the judgment of a subject or a health care professional and can be subjective (e.g., opinion) or objective (e.g., measurable by a test or diagnostic method).

In another embodiment, any of the above methods of treatment comprises the further step of co-administering to the subject in need thereof one or more additional therapeutic agents. The choice of additional therapeutic agent may be made from any additional therapeutic agent known to be useful for co-administration with volinanserin. The choice of additional therapeutic agent is also dependent upon the particular disease or condition to be treated. Examples of additional therapeutic agents that may be employed in the methods of this invention are those set forth above for use in combination compositions comprising a compound of this invention and an additional therapeutic agent.

In particular, the combination therapies of this invention include co-administering a compound of a compound of structural formula (I) (e.g., of an embodiment or aspect of embodiment thereof described herein) or structural formula (II), or pharmaceutically acceptable salt thereof, and one or more additional therapeutic agents to a subject in need thereof for treatment of the following conditions (with the particular additional therapeutic agent indicated in parentheses following the indication): depression (escitalopram).

The term “co-administered,” as used herein, means that the additional therapeutic agent may be administered together with a compound of this invention as part of a single dosage form (such as a composition of this invention comprising a compound of the invention and an additional therapeutic agent as described above) or as separate, multiple dosage forms. Alternatively, the additional agent may be administered prior to, consecutively with, or following the administration of a compound of this invention. In such combination therapy treatment, both the compounds of this invention and the additional therapeutic agent(s) are administered by conventional methods. The administration of a composition of this invention, comprising both a compound of the invention and an additional therapeutic agent, to a subject does not preclude the separate administration of that same therapeutic agent, any other additional therapeutic agent or any compound of this invention to said subject at another time during a course of treatment.

Effective amounts of these additional therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the additional therapeutic agent's optimal effective-amount range.

In one embodiment of the invention, where an additional therapeutic agent is administered to a subject, the effective amount of the compound of this invention is less than its effective amount would be where the additional therapeutic agent is not administered. In another embodiment, the effective amount of the additional therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of structural formula (I) (e.g., of an embodiment or aspect of embodiment thereof described herein) or structural formula (II), or pharmaceutically acceptable salt thereof, alone or together with one or more of the above-described additional therapeutic agents in the manufacture of a medicament, either as a single composition or as separate dosage forms, for treatment in a subject of a disease, disorder or symptom set forth above. Another aspect of the invention is a compound of structural formula (I) (e.g., of an embodiment or aspect of embodiment thereof described herein) or structural formula (II), or pharmaceutically acceptable salt thereof, for use in the treatment in a subject of a disease, disorder or symptom thereof delineated herein.

### EXAMPLES

### Example 1. Synthesis of (R)-(2,3-bis(methoxy-d₃)phenyl)(1-(4-fluorophenethyl)piperidin-4-yl)methanol (Compound 147)

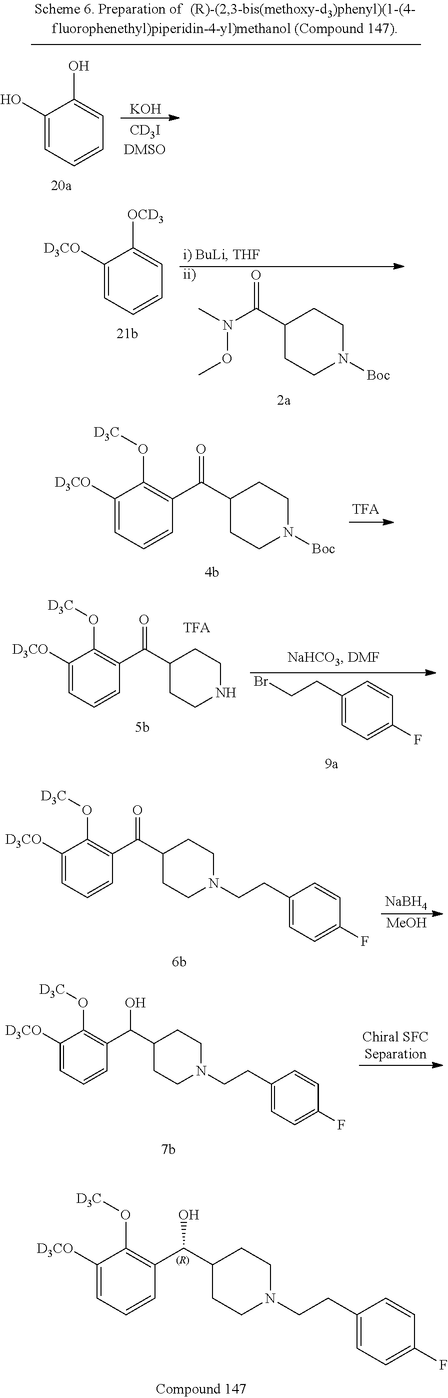

Step 1. 1,2-bis(methoxy-d₃)benzene (21b). To a solution of 1,2-dihydroxybenzene (20a) (30 g, 272.5 mmol) in anhydrous DMSO (250 mL) at room temperature was added KOH (61.2 g, 1090 mmol) followed by methyl iodide-d₃ (42.4 mL, 681.1 mmol, Sigma Aldrich, >99.5% atom D). The reaction mixture was stirred at room temperature overnight. The reaction mixture was diluted with water (800 mL) and extracted with CH₂Cl₂ (4×600 mL). The combined organic layers were washed with water (3×1L), dried (Na₂SO₄), filtered and concentrated under reduced pressure. The residue was dried (vacuum oven) to give 21b (36.4 g, 92%) as a yellow oil.

Step 2. tert-butyl 4-(2,3-bis(methoxy-d₃)benzoyl)piperidine-1-carboxylate (4b). A solution of 2.5M n-butyllithium in hexanes (50 mL, 125 mmol) was slowly added to a solution of 21b (18 g, 125 mmol) in THE (230 mL) at 0° C. The reaction mixture was warmed to room temperature, stirred 2 h then re-cooled to 0° C. A solution of 2a (34.0 g, 125 mmol) in THE (400 mL) was precooled 0° C. and slowly added to the reaction mixture. The reaction mixture was warmed to room temperature and stirred overnight. The reaction mixture was quenched with saturated aqueous NH₄Cl solution (400 mL). The layers were separated and the aqueous layer extracted with EtOAc (3×600 mL). The combined organic layers were washed with saturated brine (1×600 mL), dried (Na₂SO₄), filtered and concentrated to a yellow oil. The crude material was purified by chromatography in three batches (Interchim automated chromatography system, SorbTech 330 g silica cartridge, eluting with a gradient of 5-20% EtOAc in hexanes) to give 4b (26.6 g, 60%) as a clear oil.

Step 3. (2,3-bis(methoxy-d₃)phenyl)(piperidin-4-yl)methanone (5b). A mixture of 4b (26.6 g, 75 mmol) and trifluoroacetic acid (172 mL, 2240 mmol) was stirred at room temperature for 30 min. The reaction mixture was concentrated under reduced pressure to give a clear oil. Et₂O (800 mL) was added to the mixture, yielding a white precipitate, which was filtered to give 5b (26.5 g, 95%) as a white solid.

Step 4. (2,3-Bis(methoxy-d₃)phenyl)(1-(4-fluorophenethyl)piperidin-4-yl)methanone (6b). To a solution of 5b (5.0 g, 14.2 mmol) in anhydrous DMF (66 mL) at room temperature was added sodium bicarbonate (3.0 g, 35.5 mmol) followed by 9a (2.88 g, 14.2 mmol). The reaction mixture was heated at 90° C. for 3 h then concentrated under reduced pressure. The residue was diluted with EtOAc (100 mL) then washed with water (3×100 mL) and saturated brine (100 mL). The organic layer was concentrated under reduced pressure and the residue purified by chromatography (Interchim automated chromatography system, Biotage Sfar 60 g silica cartridge, eluting with a gradient of 0-5% MeOH in CH₂Cl₂) to give 6b (2.96 g, 55%) as a brown oil.

Step 5. (2,3-Bis(methoxy-d₃)phenyl)(1-(4-fluorophenethyl)piperidin-4-yl)methanol (7b). Sodium borohydride (0.24 g, 6.37 mmol) was added to a solution of 6b (0.80 g, 2.12 mmol) in MeOH (30 mL) at 0° C. The reaction mixture was warmed to room temperature and stirred for 16 h. The reaction mixture was cooled to 0° C. and sodium borohydride (0.16 g, 4.24 mmol) was added. The reaction mixture was warmed to room temperature and stirred for 16 h. The reaction mixture was concentrated under reduced pressure and the residue partitioned between water (50 mL) and EtOAc (50 mL). The layers were separated and the organic layer extracted with EtOAc (2×50 mL). The combined organic layers were concentrated under reduced pressure and purified by chromatography (Interchim automated chromatography system, Interchim 25 g HP silica cartridge, eluting with a gradient of 0-10% MeOH in CH₂Cl₂) to give racemic 7b (0.51 g, 64%) as a white solid.

Chiral separation of (R)-(2,3-bis(methoxy-d₃)phenyl)(1-(4-fluorophenethyl)piperidin-4-yl)methanol (Compound 147). 7b (0.44 g) was purified by chiral SFC (Chiralpak AD-H, 250×20 mm, 5 mm; 25% EtOH (0.1% diethylamine/CO₂, 100 Bar; flow rate 65 mL/min; wavelength 220 nm), to yield the early eluting (R) enantiomer as a clear oil (220 mg).

The resulting clear oil was triturated with CH₂Cl₂ and hexane to give compound 147 (212 mg, 96%) as a white solid.

¹H NMR (400 MHz, CDCl₃) δ 1.26-1.53 (m, 3H), 1.68 (td, J=3.9, 7.8, 11.6 Hz, 1H), 1.81-2.02 (m, 2H), 2.04-2.11 (m, 1H), 2.39 (br s, 1H), 2.46-2.58 (m, 2H), 2.70-2.82 (m, 2H), 2.92 (br d, J=12.0 Hz, 1H), 3.07 (br d, J=11.2 Hz, 1H), 4.63 (d, J=8.1 Hz, 1H), 6.81-7.00 (m, 4H), 7.01-7.07 (m, 1H), 7.13 (t, J=6.1 Hz, 2H). LCMS (method: SorbTech C₁₈ AQ, 2.1×50 mm, 3 μm; 5-95% acetonitrile/water with 0.1% formic acid in 14 min, with 4 min hold; flow rate: 0.7 mL/min; wavelength: 210 nm): retention time: 4.7 min; 99.3% purity; (EI-MS): m/z=380.2 ([M+H]⁺).

Chiral HPLC (Chiralpak IG column, 150 mm×4.6 mm, 3 μm, method: 100% EtOH; flow rate: 0.8 mL/min; wavelength: 230 nm): retention times: (R) isomer: 4.1 min, (S) isomer: 5.3 min; 99.9% ee.

Optical rotation [α]_{D}²⁰ (2.14 g/100 mL MeOH)=+19.1°.

### Example 2. Synthesis of (R)-(1-(4-fluorophenethyl)piperidin-4-yl)(2-methoxy-3-(methoxy-d₃)phenyl)methanol (Compound 115)

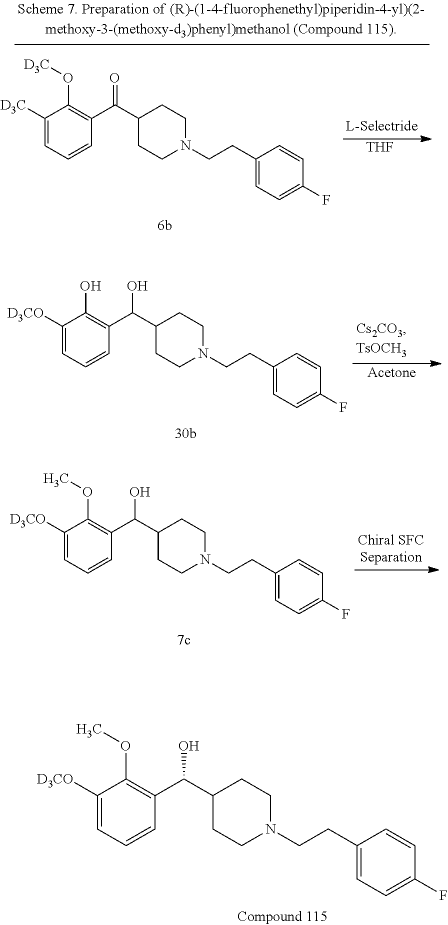

Step 1. 2-((1-(4-Fluorophenethyl)piperidin-4-yl)(hydroxy)methyl)-6-(methoxy-d₃) phenol (30b). A solution of 1.0M L-Selectride in THE (27 mL, 27 mmol) was added to a solution of 6b (2.5 g, 7 mmol) in anhydrous THE (100 mL) at 0° C. The reaction mixture was stirred at 0° C. for 2 h then heated at 70° C. overnight. The reaction mixture was cooled to 0° C. and quenched with water (150 mL). The layers were separated and the aqueous layer was extracted with Et₂O (2×150 mL). The combined organic layers were washed with saturated brine solution, dried (Na₂SO₄), filtered and concentrated under reduced pressure. The residue was purified by chromatography (Interchim automated chromatography system, Biotage 100 g silica cartridge, eluting with a gradient of 0-10% 0.3M ammonia in MeOH in CH₂Cl₂) to give 30b (0.8 g, 33%) as a white solid.

Step 2. (1-(4-Fluorophenethyl)piperidin-4-yl)(3-(methoxy-d₃)-2-methoxyphenyl)-methanol (7c). Cesium carbonate (1.2 g, 3.8 mmol) was added to a solution of 30b (680 mg, 1.9 mmol) in acetone (60 mL) at room temperature. Methyl-4-methylbenzenesulfonate (321 mg, 1.7 mmol) was added at room temperature over 4 h in four portions. The reaction mixture was stirred at room temperature for 1 h, filtered through celite (10 g), and the filter cake was washed with CH₂Cl₂ (2×50 mL). The filtrate was concentrated under reduced pressure. The residue was purified by chromatography (Interchim automated chromatography system, Biotage 55 g KP—NH cartridge, eluting with a gradient of 0-10% MeOH in CH₂Cl₂). The obtained material was re-purified as above to give racemic 7c (458 mg, 65%) as a clear oil.

Chiral separation of (R)-(1-(4-fluorophenethyl)piperidin-4-yl)(2-methoxy-3-(methoxy-d₃)phenyl)methanol (Compound 115). 7c (0.45 g) was purified by chiral SFC (AD-H, 250×20 mm, 5 mm; 25% EtOH (0.1% diethylamine/CO₂, 100 Bar; flow rate 60 mL/min; wavelength 220 nm), to yield the early eluting (R) enantiomer as a clear oil (230 mg). The resulting clear oil was triturated with CH₂Cl₂ and hexane to give Compound 115 (184 mg) as a white solid.

¹H NMR (400 MHz, CDCl₃) δ 1.24-1.33 (m, 1H), 1.33-1.43 (m, 1H), 1.44-1.54 (m, 1H), 1.68 (tdt, J=3.9, 7.8, 11.6 Hz, 1H), 1.91 (dt, J=2.8, 11.5 Hz, 1H), 1.99 (dt, J=2.4, 11.6 Hz, 1H), 2.05-2.11 (m, 1H), 2.37 (br s, 1H), 2.49-2.56 (m, 2H), 2.73-2.81 (m, 2H), 2.93 (br d, J=10.9 Hz, 1H), 3.07 (br d, J=11.2 Hz, 1H), 3.87 (s, 3H), 4.63 (d, J=8.1 Hz, 1H), 6.84 (dd, J=1.5, 8.1 Hz, 1H), 6.89 (dd, J=1.5, 7.8 Hz, 1H), 6.91-6.98 (m, 2H), 7.02-7.08 (m, 1H), 7.10-7.16 (m, 2H).

LCMS (method: Atlantis T3 column, 2.1×50 mm, 3 μm; 5-95% acetonitrile/water with 0.1% formic acid in 14 min, with 4 min hold; flow rate: 0.7 mL/min; wavelength: 210 nm): retention time: 4.5 min; 98.8% purity; (EI-MS): m/z=377.2 ([M+H]⁺)

Chiral HPLC (Chiralpak IG column, 250 mm×4.6 mm, 5 μm, method: 100% EtOH; flow rate: 1.0 mL/min; wavelength: 230 nm): retention times: (R) isomer: 4.8 min, (S) isomer: 5.8 min; 98.9% ee.

### Example 3. Synthesis of (R)-(2,3-bis(methoxy-d3)phenyl)(1-(4-fluorophenethyl)piperidin-4-yl)methan-d-ol (Compound 148)

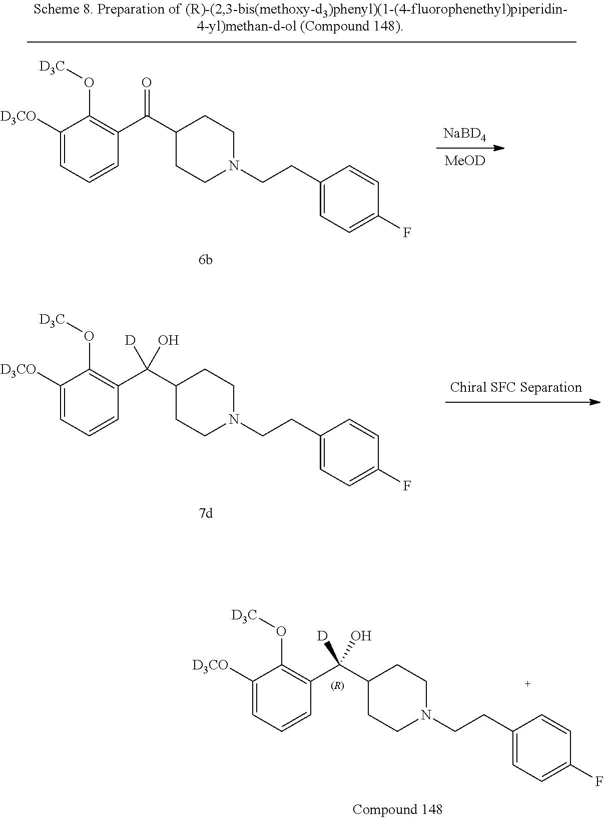

Step 1. (2,3-Bis(methoxy-d₃)phenyl)(1-(4-fluorophenethyl)piperidin-4-yl)methan-d-ol (7d). Sodium borodeuteride (0.64 g, 15.12 mmol, CIL, 99% D4) was added to a solution of 6b (1.90 g, 5.04 mmol) in MeOD (70 mL, Aldrich, 99.5 atom % D) at 0° C. The reaction mixture was warmed to room temperature and stirred for 16 h. The reaction mixture was concentrated under reduced pressure and the residue partitioned between water (50 mL) and EtOAc (50 mL). The layers were separated and the aqueous layer was extracted with EtOAc (2×50 mL). The combined organic layers were concentrated under reduced pressure to give 7d (2.4 g, quantitative) as a yellow oil.

Chiral separation. (R)-(2,3-bis(methoxy-d₃)phenyl)(1-(4-fluorophenethyl)piperidin-4-yl)methan-d-ol (Compound 148). 7d (830 mg) was purified by chiral SFC (AD-H, 250×20 mm, 5 mm; 25% EtOH (0.1% diethylamine/CO₂, 100 Bar; flow rate 65 mL/min; wavelength 220 nm), to yield the early eluting (R) enantiomer as a clear oil (398 mg).

The resulting clear oil was triturated with CH₂Cl₂ and hexane to give Compound 148 (286 mg) as a white solid.

¹H NMR (400 MHz, CDCl₃) δ 1.21-1.32 (m, 1H), 1.32-1.42 (m, 1H), 1.43-1.52 (m, 1H), 1.57 (s, 4H), 1.67 (tt, J=3.8, 7.8, 11.6 Hz, 1H), 1.89 (dt, J=2.9, 11.5 Hz, 1H), 1.97 (dt, J=2.4, 11.7 Hz, 1H), 2.08 (br d, J=13.1 Hz, 1H), 2.31 (br s, 1H), 2.49-2.54 (m, 2H), 2.73-2.78 (m, 2H), 2.92 (br d, J=10.8 Hz, 1H), 3.07 (br d, J=11.5 Hz, 1H), 3.81-3.85 (m, 1H), 4.60-4.65 (m, 1H), 6.84 (dd, J=1.4, 8.1 Hz, 1H), 6.89 (dd, j=1.5, 7.8 Hz, 1H), 6.95 (t, J=8.7 Hz, 2H), 7.04 (t, J=7.9 Hz, 1H), 7.13 (dd, J=5.5, 8.5 Hz, 2H).

LCMS (method: SorbTech C18AQ column, 2.1×50 mm, 3 μm; 5-95% acetonitrile/water with 0.1% formic acid in 14 min, with 4 min hold; flow rate: 0.7 mL/min; wavelength: 210 nm): 4.6 min; 99.6% purity; (EI-MS): m/z=381.3 ([M+H]⁺).

Chiral HPLC (Chiralpak IG column, 150 mm×4.6 mm, 3 μm, method: 100% EtOH; flow rate: 0.8 mL/min; wavelength: 230 nm): retention times: (R) isomer: 4.1 min, (S) isomer: 5.3 min; 99.7% ee.

### Example 4. Synthesis of (R)-(1-(4-fluorophenethyl)piperidin-4-yl)(3-methoxy-2-(methoxy-d₃)phenyl)methanol (Compound 131)

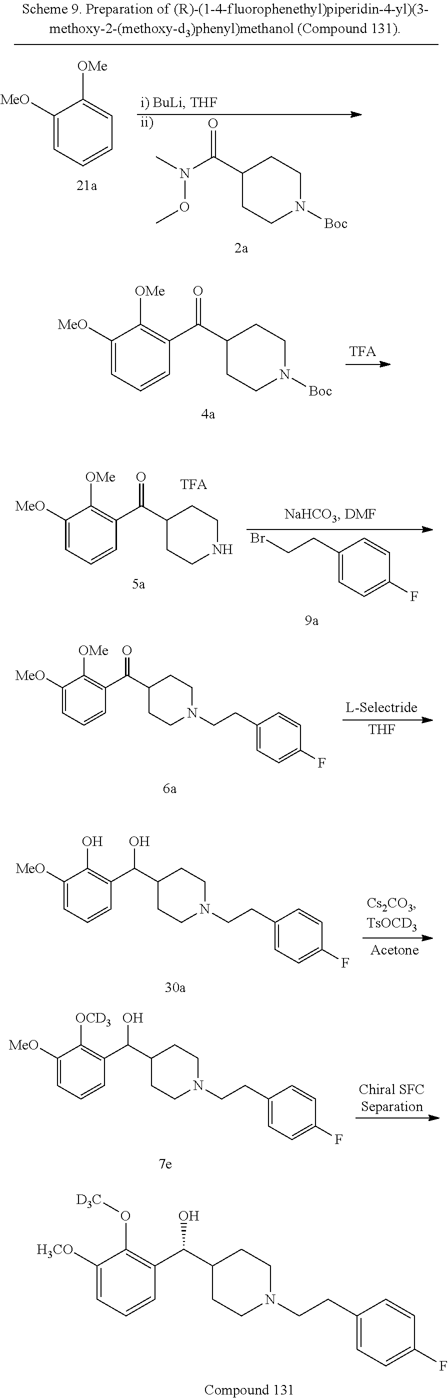

Step 1. tert-Butyl 4-(2,3-bismethoxybenzoyl)piperidine-1-carboxylate (4a). A solution of 2.5M n-butyllithium in hexanes (7.8 mL, 19.5 mmol) was added to a solution of 21a (2.7 g, 19.5 mmol) in anhydrous THF (30 mL) at 0° C. The reaction mixture was warmed to room temperature for 2 h then re-cooled to 0° C. A precooled 0° C. solution of 2a (5.3 g, 19.5 mmol) in anhydrous THE (50 mL) was added slowly at 0° C. The reaction mixture was warmed to room temperature and stirred overnight. The reaction mixture was quenched with saturated NH₄Cl solution (100 mL). The layers were separated and the aqueous layer was extracted with EtOAc (3×120 mL). The combined organic layers were washed with saturated brine solution, dried (Na₂SO₄), filtered and concentrated under reduced pressure. The residue was purified by chromatography (Interchim automated chromatography system, SorbTech 220 silica cartridge, eluting with a gradient of 5-20% EtOAc in hexanes) to give 4a (4.05 g, 60%) as a clear oil.

Step 2. (2,3-Bismethoxyphenyl)(piperidin-4-yl)methanone trifluoroacetate salt (5a). Trifluoroacetic acid (44 mL, 576 mmol) was added at 0° C. to 4a (6.7 g, 19 mmol). The reaction mixture was stirred at room temperature for 30 minutes then concentrated under reduced pressure to give a clear oil. Et₂O (100 mL) was added and the mixture was stirred at room temperature for 2 h. The solid was filtered and washed with Et₂O (50 mL) to give 5a (5.9 g, 90%) as a white solid.

Step 3. (2,3-Dimethoxyphenyl)(1-(4-fluorophenethyl)piperidin-4-yl)methanone (6a). NaHCO₃ (3.6 g, 43 mmol) and 9a (3.5 g, 17 mmol) were added to a solution of 5a (5.9 g, 17 mmol) in DMF (80 mL) at room temperature. The reaction mixture was heated at 900 overnight. The reaction mixture was cooled to room temperature and concentrated under reduced pressure. The residue was adsorbed onto Celite (25 g) then purified by chromatography (Interchim automated chromatography system, Biotage 100 g silica cartridge, eluting with a gradient of 0-10% MeOH in CH₂Cl₂) to give 6a (4.3 g, 68%) as a dark brown oil.

Step 4. 2-((1-(4-Fluorophenethyl)piperidin-4-yl)(hydroxy)methyl)-6-methoxyphenol (30a). A 1.0 solution of L-Selectride in THE (46 mL, 46 mmol) was added to a solution of 6a (4.3 g, 12 mmol) in anhydrous THE (200 mL) at 0° C. The reaction mixture was stirred at 0° C. for 2 h then heated at 70° C. overnight. The reaction mixture was cooled to 0° C. and diluted with water (150 mL). The layers were separated and the aqueous layer extracted with Et₂O (2×150 mL). The combined organic layers were washed with saturated brine solution and concentrated under reduced pressure. The residue was purified by chromatography (Interchim automated chromatography system, Biotage 100 g silica cartridge, eluting with a gradient of 0-10% 0.3M ammonia/MeOH in CH₂Cl₂) to give 30a (2.9 g, 70%) as a yellow solid.

Step 5. (1-(4-Fluorophenethyl)piperidin-4-yl)(3-methoxy-2-(methoxy-d₃)phenyl)-methanol (7e). Cesium carbonate (564 mg, 1.7 mmol) was added to a solution of 30a (310 mg, 0.86 mmol) in acetone (50 mL) at room temperature. Methyl-d₃-4-methylbenzenesulfonate (151 mg, 0.8 mmol, CDN, 99.5 atom % D) was added to the reaction mixture at room temperature over 4 h in four equal portions. The reaction mixture was stirred at room temperature for 1 h then filtered through Celite (10 g), washing the filter cake with CH₂Cl₂ (2×50 mL). The filtrate was concentrated under reduced pressure. The residue was purified by chromatography (Interchim automated chromatography system, Biotage 110 g KP—NH cartridge, eluting with a gradient of 0-10% MeOH in CH₂Cl₂) to give 7e (0.2 g, 62%) as a clear oil.

Chiral separation of (R)-(1-(4-fluorophenethyl)piperidin-4-yl)(3-methoxy-2-(methoxy-d₃)phenyl)methanol (Compound 131). 7e (390 mg) was purified by chiral SFC (AD-H, 250×20 mm, 5 mm; 25% EtOH (0.1% diethylamine/CO₂, 100 Bar; flow rate 60 mL/min; wavelength 220 nm), to yield the early eluting (R) enantiomer as a clear oil (180 mg).

The resulting clear oil was triturated with CH₂Cl₂ and hexane to give Compound 131 (148 mg, 82% recovery) as a white solid.

¹H NMR (400 MHz, CDCl₃) δ 1.24-1.33 (m, 1H), 1.34-1.43 (m, 1H), 1.44-1.54 (m, 1H), 1.68 (tdt, J=3.9, 7.8, 11.6 Hz, 1H), 1.79 (br s, 1H), 1.90 (dt, J=2.9, 11.4 Hz, 1H), 1.98 (dt, J=2.4, 11.7 Hz, 1H), 2.08 (quint, J=2.8, 13.1 Hz, 1H), 2.37 (br s, 1H), 2.48-2.56 (m, 2H), 2.71-2.81 (m, 2H), 2.93 (br d, J=11.1 Hz, 1H), 3.07 (br d, J=11.4 Hz, 1H), 3.87 (s, 3H), 4.63 (d, J=8.1 Hz, 1H), 6.84 (dd, J=1.5, 8.2 Hz, 1H), 6.89 (dd, J=1.5, 7.8 Hz, 1H), 6.91-6.98 (m, 2H), 7.01-7.07 (m, 1H), 7.10-7.17 (m, 2H).

LCMS (method: Atlantis T3 column, 2.1×50 mm, 3 μm; 5-95% acetonitrile/water with 0.1% formic acid in 14 min, with 4 min hold; flow rate: 0.7 mL/min; wavelength: 210 nm): retention time: 4.6 min; 99.9% purity; (EI-MS): m/z=377.2 ([M+H]⁺)

Chiral HPLC (Chiralpak IG column, 250 mm×4.6 mm, 5 μm, method: 100% EtOH; flow rate: 1.0 mL/min; wavelength: 230 nm): retention times: (R) isomer: 4.8 min, (S) isomer: 5.8 min; 99.9% ee.

### Example 5. Synthesis of (R)-(2,3-bis(methoxy-d₃)phenyl)(1-(2-(4-fluorophenyl)ethyl-1,1-d₂)piperidin-4-yl)methanol (Compound 151)

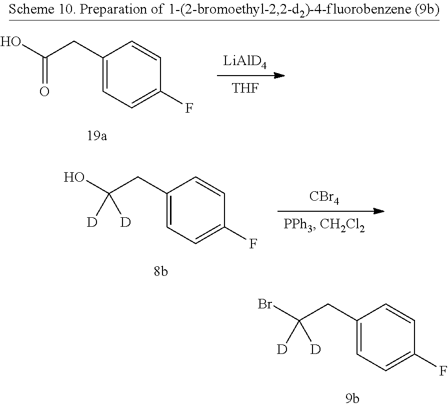

Step 1. 2-(4-Fluorophenyl)ethan-1,1-d₂-1-ol (8b). 19a (5.0 g, 32.45 mmol) was dissolved in MeOD (8 mL, Cambridge Isotope, 99.8 atom % D) and concentrated under reduced pressure, then repeated twice. The residue was dissolved in anhydrous THE (20 mL) and added at 0° C. to a suspension of lithium aluminum deuteride (1.36 g, 32.45 mmol, Boc Sciences, 98 atom % D) in anhydrous THE (50 mL). The reaction mixture was warmed to room temperature stirred for 1 h then heated at reflux for 4 h. The reaction mixture was cooled to room temperature and quenched with water (1.5 mL), 15% sodium hydroxide solution (2 mL) then water (3 mL). The mixture was filtered through a pad of Celite (20 g) and the filtrate concentrated under reduced pressure. The crude product was purified by chromatography (Interchim automated chromatography system, RediSep 80 g silica cartridge, eluting with a gradient 0-25% acetone in hexanes) to give 8b (4.0 g, 87%) as a yellow oil.

Step 2. 1-(2-Bromoethyl-2,2-d₂)-4-fluorobenzene (9b). Carbon tetrabromide (5.08 g, 15.32 mmol) was added to a solution of 8b (1.74 g, 12.26 mmol) in CH₂Cl₂ (25 mL) at 0° C. followed by triphenylphosphine (4.82 g, 18.39 mmol). The reaction mixture was stirred at 0° C. for 1 h, then diluted with diethyl ether (100 mL) and hexane (40 mL) and stirred for 40 min, resulting in formation of a white precipitate. The suspension was filtered through a pad of Celite (20 g) to give a clear filtrate which was concentrated under reduced pressure. The crude product was purified by chromatography (Interchim automated chromatography system, Biotage 100 g silica gel cartridge, eluting with a gradient of 0-10% EtOAc in hexane) to give 9b (1.57 g, 63%) as a clear oil.

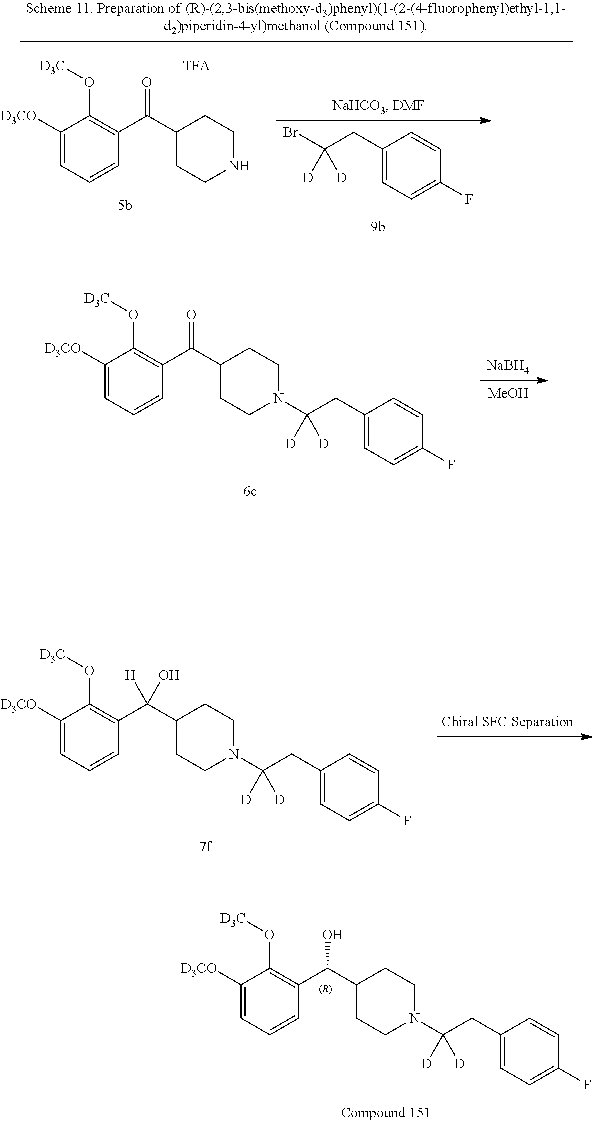

Step 3. (2,3-Bis(methoxy-d₃)phenyl)(1-(2-(4-fluorophenyl)ethyl-1,1-d₂)piperidin-4-yl)-methanone (6c). Sodium bicarbonate powder (0.89 g, 10.61 mmol) followed by a solution of 9b (0.87 g, 4.24 mmol) in anhydrous DMF (1 mL) were added to a solution of 5b (1.5 g, 4.24 mmol) in anhydrous DMF (21 mL) at rt. The reaction mixture was heated at 90° C. for 3 h, then concentrated under reduced pressure. Water (30 mL) was added and the mixture was extracted with EtOAc (3×30 mL). The combined organic layers were washed with water (30 mL), saturated brine (30 mL), dried (Na₂SO₄), filtered and concentrated under reduced pressure. The crude product was purified by chromatography (Interchim automated chromatography system, SorbTech 80 g silica gel cartridge, eluting with a gradient of 0-5% MeOH in CH₂Cl₂) to give 6c (0.58 g, 36%) as a yellow oil.

Step 4. (2,3-Bis(methoxy-d₃)phenyl)(1-(2-(4-fluorophenyl)ethyl-1,1-d₂)piperidin-4-yl)-methanol (7f). Sodium borohydride (0.173 g, 4.59 mmol) was added in one portion to a solution of 6c (0.58 g, 1.53 mmol) in MeOH (20 mL) at 0° C. The reaction mixture was warmed to room temperature and stirred overnight. The reaction mixture was concentrated under reduced pressure and the residue partitioned between water (20 mL) and EtOAc (30 mL). The layers were separated and the aqueous layer was extracted with EtOAc (2×30 mL). The combined organic layers were dried (Na₂SO₄), filtered and concentrated under reduced pressure to give 7f (0.46 g, 80%) as a white solid.

Chiral separation. (R)-(2,3-bis(methoxy-d₃)phenyl)(1-(2-(4-fluorophenyl)ethyl-1,1-d₂)piperidin-4-yl)methanol (Compound 151) 7f (437 mg) was purified by chiral SFC (AD-H, 250×20 mm, 5 mm; 25% EtOH (0.1% diethylamine/CO₂, 100 Bar; flow rate 60 mL/min; wavelength 220 nm), to yield the early eluting (R) enantiomer as a clear oil (230 mg).

The resulting clear oil was triturated with CH₂Cl₂ and hexane to give Compound 151 (220 mg) as a white solid.

¹H NMR (400 MHz, CDCl₃) δ 1.22-1.34 (m, 2H), 1.38 (s, 1H), 1.48 (d, J=24.5 Hz, 1H), 1.68 (d, J=8.0 Hz, 1H), 1.89 (d, J=20.3 Hz, 1H), 1.98 (d, J=23.3 Hz, 1H), 2.08 (d, J=18.5 Hz, 1H), 2.31 (s, 1H), 2.74 (s, 2H), 2.92 (d, J=11.6 Hz, 1H), 3.06 (d, J=11.9 Hz, 1H), 4.59-4.67 (m, 1H), 6.84 (d, J=9.5 Hz, 1H), 6.89 (d, J=8.9 Hz, 1H), 6.95 (t, J=8.7 Hz, 2H), 7.04 (t, J=7.9 Hz, 1H), 7.10-7.16 (m, 2H).

LCMS (method: SorbTech C18AQ column, 2.1×50 mm, 3 μm; 5-95% acetonitrile/water with 0.1% formic acid in 14 min, with 4 min hold; flow rate: 0.7 mL/min; wavelength: 210 nm): retention time: 4.6 min; 98.6% purity; (EI-MS): m/z=382.2 ([M+H]⁺

Chiral HPLC (Chiralpak IG column, 150 mm×4.6 mm, 3 μm, method: 100% EtOH; flow rate: 0.3 mL/min; wavelength: 230 nm): retention times: (R) isomer: 10.9 min, (S) isomer: 14.2 min; 99.7% ee

### Example 6. Synthesis of (R)-(2,3-bis(methoxy-d₃)phenyl)(1-(2-(4-fluorophenyl)ethyl-1,1,2,2-d₄)piperidin-4-yl)methanol (Compound 159)

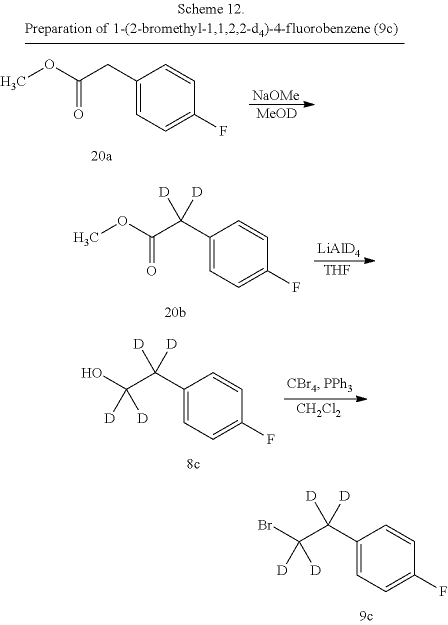

Step 1. Methyl 2-(4-fluorophenyl)acetate-d₂ (20b). A freshly-prepared 2.11M sodium methoxide solution (2.82 mL, 5.9 mmol) in MeOD was added at room temperature to a solution of 20a (10 g, 59.5 mmol) in MeOD (100 mL). The reaction mixture was stirred at room temperature overnight then concentrated under reduced pressure to give a white semi-solid. Additional MeOD (110 mL) and freshly-prepared 2.11M sodium methoxide solution in MeOD (2.82 mL, 5.9 mmol) were added at room temperature then the reaction mixture was stirred overnight. This process was repeated for a total of 4 cycles. The reaction mixture was concentrated under reduced pressure to give 20b (11.50 g, quantitative yield).

Step 2. 2-(4-Fluorophenyl)ethan-1,1,2,2-d₄-1-ol (8c). A suspension of 20b (10 g, 58.7 mmol) in anhydrous THE (50 mL) was slowly added to a suspension of lithium aluminum deuteride (3.69 g, 88.0 mmol, Boc Sciences, 98 atom % D) in anhydrous THE (100 mL) at 0° C. The reaction mixture was warmed to room temperature, stirred for 1 h then heated at reflux for 4 h. The reaction mixture was cooled to room temperature then quenched with water (3 mL), 15% NaOH solution (4 mL) then water (6 mL). THE was added as needed as the mixture became very thick during quenching. The mixture was then filtered through a pad of Celite (20 g) and the filtrate concentrated under reduced pressure. The crude product was purified by chromatography (Interchim automated chromatography system, Biotage 350 g silica gel cartridge, eluting with a gradient of 0-25% acetone in hexanes) to give 8c (7.06 g, 83%) as a clear oil.

Step 3. 1-(2-Bromoethyl-1,1,2,2-d₄)-4-fluorobenzene (9c). Carbon tetrabromide (23.95 g, 72.0 mmol) was added to a solution of 8c (8.33 g, 58.0 mmol) in CH₂Cl₂ (140 mL) at 0° C. followed by addition of triphenylphosphine (22.73 g, 86.6 mmol). The reaction mixture was stirred at 0° C. for 2 h, then concentrated under reduced pressure to a yellow oil. The oil was diluted with Et₂O (400 mL) and stirred for 40 minutes to yield a white suspension. The suspension was filtered through a pad of Celite (20 g) to give a clear filtrate which was concentrated under reduced pressure. Hexanes (300 mL) was added and the mixture was stirred for 15 minutes to give more white precipitate. The precipitate was filtered through a fine-fritted funnel and the filtrate concentrated under reduced pressure. The residue was purified by chromatography (Biotage automated chromatography system, Biotage 350 g silica gel cartridge, eluting with a gradient of 0-25% EtOAc in hexanes) to give 9c (7.7 g, 64%) as a clear oil.

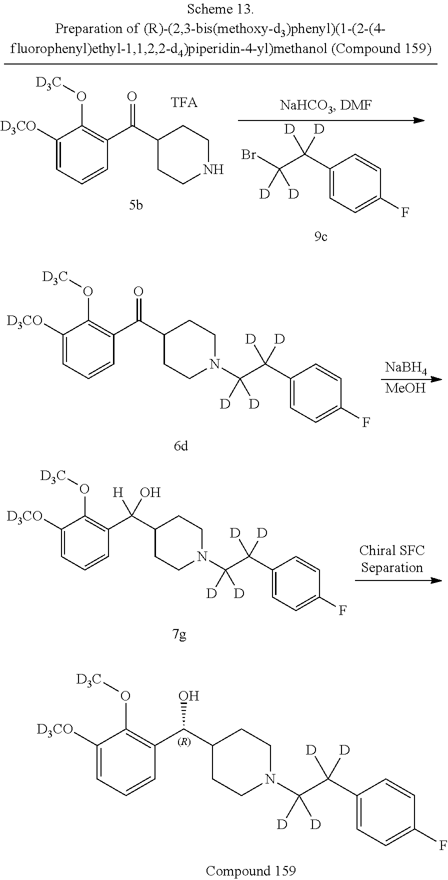

Step 4. (2,3-Bis(methoxy-d₃)phenyl)(1-(2-(4-fluorophenyl)ethyl-1,1,2,2-d₄)piperidin-4-yl)methanone (6d). Sodium bicarbonate powder (0.71 g, 8.4 mmol) followed by a solution of 9c (0.70 g, 3.35 mmol, 1 equiv) in DMF (3 mL) were added to a solution of 5b (1.2 g, 3.35 mmol) in DMF (15 mL) at rt. The reaction mixture was heated at 90° C. for 2 h, then cooled to room temperature and concentrated under reduced pressure. The residue was diluted with water (35 mL) and extracted with EtOAc (3×35 mL). The combined organic layers were washed with saturated brine (50 mL), water (30 mL), dried (Na₂SO₄), filtered and concentrated under reduced pressure. The crude product was purified by chromatography (Biotage automated chromatography system, Biotage 50 g silica gel cartridge, eluting with a gradient of 0-5% MeOH in CH₂Cl₂) to give 6d (0.86 g, 68%) as a yellow oil.

Step 5. (2,3-Bis(methoxy-d₃)phenyl)(1-(2-(4-fluorophenyl)ethyl-1,1,2,2-d₄)piperidin-4-yl)methanol (7 g). Sodium borohydride (0.26 g, 6.80 mmol, 3 equiv) was added in one portion to a solution of 6d (0.86 g, 2.27 mmol, 1 equiv) in MeOH (30 mL) at 0° C. The reaction mixture was warmed to room temperature and stirred for 38 h. The reaction mixture was concentrated under reduced pressure and the residue partitioned between water (30 mL) and EtOAc (30 mL). The layers were separated and the aqueous layer was extracted with EtOAc (2×30 mL). The combined organic layers were dried (Na₂SO₄), filtered and concentrated under reduced pressure to give 7 g (0.77 g, 89%) as a white solid.

Chiral separation of (R)-(2,3-bis(methoxy-d₃)phenyl)(1-(2-(4-fluorophenyl)ethyl-1,1,2,2-d₄)piperidin-4-yl)methanol (Compound 159) 7 g (700 mg) was purified by chiral SFC (AD-H, 250×20 mm, 5 mm; 25% EtOH (0.1% diethylamine/CO₂, 100 bar; flow rate 65 mL/min; wavelength 220 nm), to yield the early eluting (R) enantiomer as a clear oil (385 mg).

The resulting clear oil was triturated with CH₂Cl₂ and hexane to give Compound 159 (270 mg) as a white solid.

¹H NMR (400 MHz, CDCl₃) δ 1.21-1.42 (m, 2H), 1.42-1.55 (m, 1H), 1.68 (dtd, J=4.0, 7.8, 11.6 Hz) (s, 2H), 1.91 (br t, J=11.0 Hz, 1H), 1.99 (br t, J=11.4 Hz, 1H), 2.08 (d, J=2.7, 13.1 Hz, 1H), 2.35 (br s, 1H), 2.93 (d, J=10.9 Hz, 1H), 3.07 (d, J=10.8 Hz, 1H), 4.63 (d, J=8.1 Hz, 1H), 6.84 (dd, J=1.6, 8.1 Hz, 1H), 6.89 (dd, J=1.4, 7.8 Hz, 1H), 6.91-6.98 (m, 2H), 7.02-7.07 (m, 1H), 7.10-7.17 (m, 2H).

LCMS (method: Atlantis T3 column, 2.1×50 mm, 3 μm; 5-95% acetonitrile/water with 0.1% formic acid in 14 min, with 4 min hold; flow rate: 0.7 mL/min; wavelength: 210 nm): retention time: 4.5 min; 99.9% purity; (ET-MS): m/z=384.3 ([M+H]⁺)

Chiral HPLC (Chiralpak IG column, 250 mm×4.6 mm, 3 μm, method: 100% EtOH; flow rate: 0.3 mL/min; wavelength: 230 nm): retention times: (R) isomer: 16.9 min, (S) isomer: 21.5 min; 99.7% ee.

### Example 7. Synthesis of (R)-(2,3-dimethoxyphenyl)(1-(4-fluorophenethyl)piperidin-4-yl-2,2,3,3,4,5,5,6,6-d₉)methanol (Compound 203)

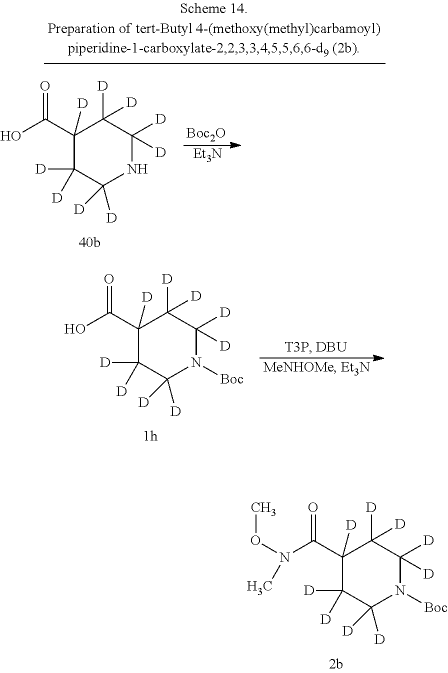

Step 1. 1-(tert-butoxycarbonyl)piperidine-4-carboxylic-2,2,3,3,4,5,5,6,6-d₉ acid (1h) MeOD (12 mL, Sigma Aldrich, 99.5 atom % D) was added to piperidine-4-carboxylic-2,2,3,3,4,5,5,6,6-d₉ acid (3 g, CDN Isotope, 98.6 atom % D). The mixture was concentrated under reduced pressure. This process was repeated two more times. Triethylamine (6.6 g, 65.0 mmol) was then added to a solution of piperidine-4-carboxylic-2,2,3,3,4,5,5,6,6-d₉ acid (3 g, 22.0 mmol) in anhydrous CH₂CO₂ (30 mL), followed by Boc anhydride (5.7 g, 26 mmol) at room temperature. The reaction mixture was stirred at this temperature overnight. The reaction mixture was concentrated under reduced pressure. THE (40 mL) was added to the residue and the mixture was acidified with 1N deuterium chloride (aq.) (30 mL, Sigma, ≥99 atom % D. The mixture was stirred for 15 minutes and then EtOAc (70 mL) was added. The layers were separated and the aqueous layer was extracted with EtOAc (2×70 mL). The organic layers were combined and washed with saturated brine in deuterium oxide (1×70 mL), dried (Na₂SO₄), filtered, concentrated under reduced pressure, and dried (vacuum oven) to give 1h (4.81 g, 93%).

Step 2. tert-Butyl 4-(methoxy(methyl)carbamoyl)piperidine-1-carboxylate-2,2,3,3,4,5,5,6,6-d₉ (2b). Triethylamine (3.1 mL, 22.0 mmol) was added to solid N,O-dimethylhydroxylamine hydrochloride (2.14 g, 22.0 mmol) at room temperature and the mixture was stirred for 1 h to give the free based N,O-dimethylhydroxylamine as an oil. DBU (3.38 g, 22.0 mmol), followed by propanephosphonic acid anhydride (1.07 g, 64.5 mmol) were added to a solution of 1h (4.81 g, 20.16 mmol) in anhydrous acetonitrile (60 mL) at 0° C. After stirring for 15 minutes, the free base N,O-dimethylhydroxylamine was added to the above reaction mixture at 0° C. and stirred at this temperature for 3 h. The reaction mixture was then concentrated under reduced pressure. EtOAc (400 mL) was added to the residue and the mixture was washed with 25% citric acid (aq.) (2×200 mL) and saturated sodium bicarbonate (2×200 mL). The organic layer was dried (Na₂SO₄), filtered, and concentrated under reduced pressure to give 2b (3.31 g, 58%) as a yellow oil.

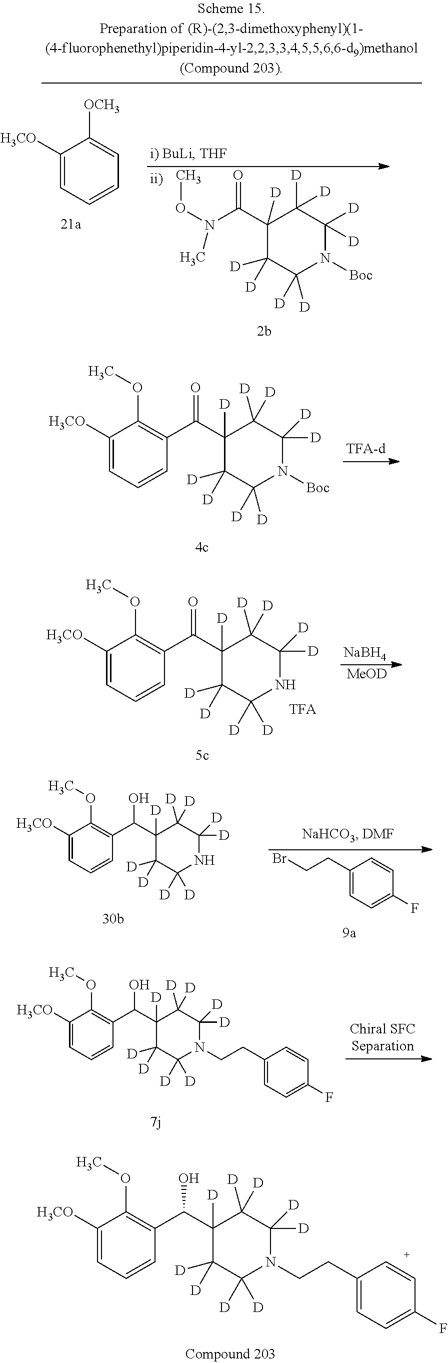

Step 1. tert-Butyl 4-(2,3-dimethoxybenzoyl)piperidine-1-carboxylate-2,2,3,3,4,5,5,6,6-d₉ (4c). A solution of 2.5M n-buthyllithium in hexanes (1.95 mL, 4.86 mmol) was added dropwise to a solution of 21a (0.64 g, 4.6 mmol, 1 equiv) in anhydrous THE (11 mL) at 0° C. After addition, the mixture was warmed to room temperature, stirred for 2 h then re-cooled to 0° C. A precooled (0° C.) solution of 2b (1.30 g, 4.6 mmol) in anhydrous THE (9 mL) was slowly added to the reaction mixture. The reaction mixture was warmed to room temperature and stirred overnight. An additional portion of 21a (0.64 g, 4.6 mmol) in anhydrous THE (11 mL) at 0° C. was treated with 2.5M n-BuLi in hexanes (1.95 mL, 4.86 mmol) then added to the reaction mixture at 0° C. The reaction mixture was warmed to room temperature and stirred overnight. The reaction mixture was quenched with saturated ND₄Cl solution in deuterium oxide (25 mL, CIL, 99.9 atom % D) and stirred 15 minutes. The layers were separated and the aqueous layer was extracted with Et₂O (3×30 mL). The combined organic layers were washed with saturated brine in deuterium oxide (50 mL), dried (Na₂SO₄), filtered and concentrated under reduced pressure to give crude 4c (2.9 g) as a yellow oil. The crude material was purified by chromatography (Biotage automated chromatography system, Biotage 100 g silica gel cartridge, eluting with a gradient of 10-15% EtOAc in hexanes), followed by reverse phase chromatography (Biotage automated chromatography system, Teledyne 100 g C18 cartridge, eluting with a 0-85% acetonitrile in water) to give 4c (0.51 g, 31%) with 22% proton incorporation alpha to the ketone. Potassium carbonate (0.3 g, 2.15 mmol) was added to a solution of the obtained 4c (0.51 g, 1.43 mmol) in a 1:1 mixture of deuterium oxide (50 mL, CIL, 99.9 atom % D) and anhydrous THE (50 mL) at room temperature. The reaction mixture was stirred for 3 d. The reaction mixture was diluted with saturated brine in deuterium oxide (20 mL, CIL, 99.9 atom % D) then extracted with CH₂Cl₂ (150 mL). The aqueous layer was extracted with CH₂Cl₂ (50 mL). The combined organic layers were washed with saturated brine in deuterium oxide (20 mL), dried (Na₂SO₄), filtered and concentrated under reduced pressure to give 4c (0.51 g).

Step 2. (2,3-Dimethoxyphenyl)(piperidin-4-yl-2,2,3,3,4,5,5,6,6-d₉)methanone trifluoroacetate salt (5c). Trifluoroacetic acid-d (7.54 g, 66.11 mmol, Sigma Aldrich, 99.5 atom % D) was added at 0° C. to 4c (0.79 g, 2.20 mmol). The reaction mixture was warmed to room temperature and stirred for 1.5 h. The reaction mixture was concentrated under reduced pressure to give a yellow oil. Et₂O (150 mL) was added and the mixture was stirred for 10 minutes to yield a white precipitate. The solid was filtered to give 5c (0.66 g, 85%) as a white solid.

Step 3. (2,3-Dimethoxyphenyl)(piperidin-4-yl-2,2,3,3,4,5,5,6,6-d₉)methanol (30b). Sodium borohydride (0.28 g, 7.45 mmol) was added in one portion to a solution of 5c (0.66 g, 1.86 mmol) in MeOD (35 mL, Sigma Aldrich, 99.5 atom % D) at 0° C. The reaction mixture was warmed to rt and stirred overnight. The reaction mixture was concentrated under reduced pressure then the residue was partitioned between saturated sodium bicarbonate in deuterium oxide (60 mL, CIL, 99.9 atom % D) and 10% MeOH in CH₂Cl₂ (100 mL). The layers were separated and the aqueous layer was extracted with 10% MeOH in CH₂Cl₂ (2×100 mL). The combined organic layers were washed with deuterium oxide (50 mL, CIL, 99.9 atom % D), dried (Na₂SO₄), filtered and concentrated under reduced pressure to give 30b (0.17 g). The combined aqueous layers from above were concentrated under reduced pressure and the residue was extracted with CH₂Cl₂ (3×100 mL). The combined organic layers were concentrated under reduced pressure to give a total of 30b (0.40 g, 83%).

Step 4. (2,3-Dimethoxyphenyl)(1-(4-fluorophenethyl)piperidin-4-yl-2,2,3,3,4,5,5,6,6-d₉)methanol (7j). Sodium bicarbonate powder (0.13 g, 1.54 mmol) followed by a solution of 9a (0.15 g, 0.77 mmol) in anhydrous DMF (3 mL) were added to a solution of 30b (0.2 g, 0.77 mmol) in anhydrous DMF (7 mL) at rt. The reaction mixture was heated at 90° C. for 2h then cooled to room temperature. The reaction mixture was concentrated under reduced pressure. The residue was diluted with water (10 mL) and extracted with EtOAc (3×25 mL). The combined organic layers were washed with saturated brine (20 mL), dried (Na₂SO₄), filtered and concentrated under reduced pressure to give 7j (0.26 g, 88%) as a yellow oil which solidified on standing.

Chiral separation. (R)-(2,3-dimethoxyphenyl)(1-(4-fluorophenethyl)piperidin-4-yl-2,2,3,3,4,5,5,6,6-d₉)methanol (Compound 203). 7j (260 mg) was purified by chiral SFC (AD-H, 250×20 mm, 5 mm; 20% EtOH (0.1% diethylamine/CO₂, 100 Bar; flow rate 60 mL/min; wavelength 220 nm), to yield the early eluting (R) enantiomer as a clear oil (130 mg).

The resulting clear oil was triturated with CH₂Cl₂ and hexane to give Compound 203 (86 mg) as a white solid.

¹H NMR (400 MHz, CDCl₃) δ 2.35 (bs, 1H), 2.49-2.55 (m, 2H), 2.72-2.83 (m, 2H), 3.87 (s, 6H), 4.63 (s, 1H), 6.85 (d, J=8.1 Hz, 1H), 6.89 (d, J=7.6 Hz, 1H), 6.96 (m, 2H), 7.05 (t, J=7.9 Hz, 1H), 7.10-7.17 (m, 2H).

LCMS (method: Atlantis T3 column, 2.1×50 mm, 3 μm; 5-95% acetonitrile/water with 0.1% formic acid in 14 min, with 4 min hold; flow rate: 0.7 mL/min; wavelength: 210 nm): retention time: 4.5 min; 99.90% purity; (ET-MS): mz=383.3 ([M+H]⁺)

Chiral HPLC (Chiralpak IG column, 250 mm×4.6 mm, 5 μm, method: 100% EtOH; flow rate: 0.3 mL/min; wavelength: 230 nm): retention times: (R) isomer: 16.6 min, (S) isomer: 21.2 min; 99.9% ee.

### Example 8. Synthesis of (R)-(2,3-dimethoxyphenyl)(1-(2-(4-fluorophenyl)ethyl-1,1-d₂)piperidin-4-yl-2,2,3,3,4,5,5,6,6-d₉)methanol (Compound 215)

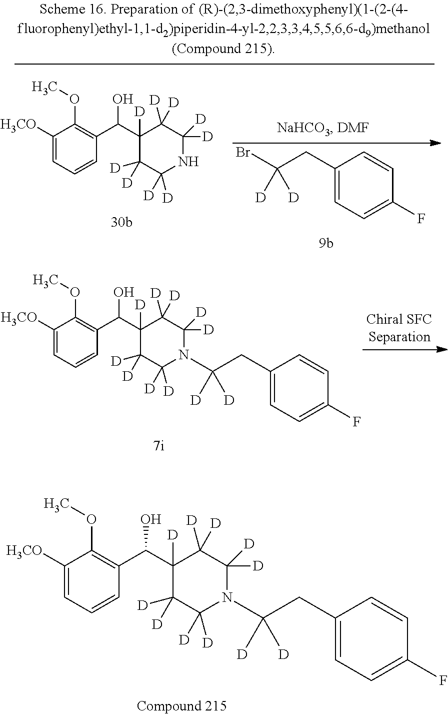

Step 1. (2,3-Dimethoxyphenyl)(1-(2-(4-fluorophenyl)ethyl-1,1-d₂)piperidin-4-yl-2,2,3,3,4,5,5,6,6-d₉)methanol (7i). Sodium bicarbonate (0.13 g, 1.6 mmol) followed by a solution of 9b (0.16 g, 0.80 mmol) in DMF (3 mL) were added to a solution of 30b (0.21 g, 0.80 mmol) in DMF (7 mL) at room temperature. The reaction mixture was heated at 90° C. for 2.5 h, cooled to room temperature and concentrated under reduced pressure. The residue was diluted with water (10 mL) and extracted with EtOAc (3×25 mL). The combined organic layers were washed with saturated brine (20 mL), dried (Na₂SO₄), filtered and concentrated under reduced pressure to give crude 7i (0.26 g, 87% yield) as a clear oil which solidified on standing.

Chiral separation of (R)-(2,3-dimethoxyphenyl)(1-(2-(4-fluorophenyl)ethyl-1,1-d₂)piperidin-4-yl-2,2,3,3,4,5,5,6,6-d₉)methanol (Compound 215). 7i (266 mg) was purified by chiral SFC (AD-H, 250×20 mm, 5 mm; 20% EtOH (0.1% diethylamine/CO₂, 100 bar; flow rate 60 mL/min; wavelength 220 nm), to yield the early eluting (R) enantiomer as a clear oil (134 mg).

The resulting clear oil was triturated with CH₂Cl₂ and hexane to give Compound 215 (105 mg) as a white solid.

¹H NMR (400 MHz, CDCl₃) δ 2.29 (br s, 1H), 2.74 (s, 2H), 3.87 (s, 6H), 4.63 (s, 1H), 6.85 (dd, J=1.5, 8.1 Hz, 1H), 6.89 (dd, J=1.2, 7.8 Hz, 1H), 6.95 (t, J=8.7 Hz, 2H), 7.05 (t, J=7.9 Hz, 1H), 7.10-7.18 (m, 2H).

LCMS (method: Atlantis T3 column, 2.1×50 mm, 3 μm; 5-95% acetonitrile/water with 0.1% formic acid in 14 min, with 4 min hold; flow rate: 0.7 mL/min; wavelength: 210 nm): retention time: 4.6 min; 99.8% purity; (EI-MS): m/z=385.3 ([M+H]⁺)

Chiral HPLC (Chiralpak IG column, 250 mm×4.6 mm, 5 μm, method: 100% EtOH; flow rate: 0.3 mL/min; wavelength: 230 nm): retention times: (R) isomer: 16.8 min, (S) isomer: 21.3 min; 99.2% ee

### Example 9. Synthesis of (R)-(2,3-bis(methoxy-d₃)phenyl)(1-(4-fluorophenethyl)piperidin-4-yl-2,2,3,3,4,5,5,6,6-d₉)methanol (Compound 347)

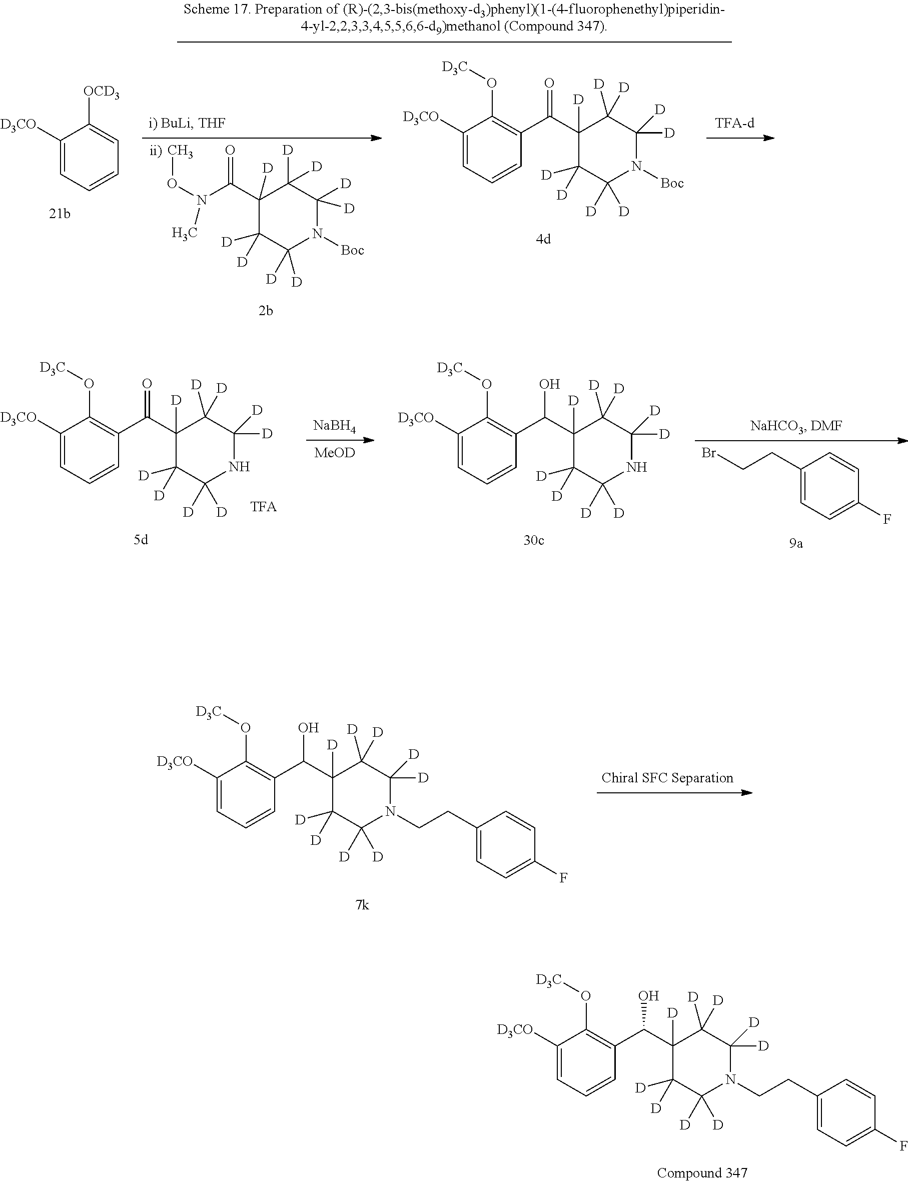

Step 1. tert-Butyl 4-(2,3-bis(methoxy-d₃)benzoyl)piperidine-1l-carboxylate-2,2,3,3,4,5,5,6,6-d₉ (4d). A solution of 2.5M n-buthyllithium in hexanes (2.89 mL, 7.23 mmol) was added dropwise to a solution of 21b (0.99 g, 6.90 mmol) in anhydrous THE (16 mL) at 0° C. After addition, the reaction mixture was warmed to room temperature, stirred for 2 h then re-cooled to 0° C. A precooled (0° C.) solution 2b (1.94 g, 6.90 mmol) in THE (14 mL) was slowly added to the reaction mixture. The reaction mixture was warmed to room temperature then stirred overnight. An additional portion of 21b (0.99 g, 6.9 mmol) in anhydrous THE (16 mL) at 0° C. was treated with 2.5M n-BuLi in hexanes (2.89 mL, 7.23 mmol) then added to the reaction mixture at 0° C. The reaction mixture was stirred overnight at room temperature. The reaction mixture was quenched with saturated ammonium chloride solution in deuterium oxide (35 mL, CIL, 99.9 atom % D) and stirred for 15 minutes. The layers were separated and the aqueous layer was extracted with EtOAc (3×100 mL). The combined organic layers were washed with saturated brine in deuterium oxide (60 mL, CIL, 99.9 atom % D), dried (Na₂SO₄), filtered and concentrated under reduced pressure to give crude 4d (3.9 g) as a yellow oil. The crude material was purified by chromatography (Biotage automated chromatography system, Biotage 100 g silica gel cartridge, eluting with a gradient of 10-15% EtOAc in hexanes), then repurified by reverse phase chromatography (Biotage automated chromatography system, Teledyne 100 g C₁₈ cartridge, eluting with a gradient of 0-85% acetonitrile in water) to give 4d (1.16 g, 46% yield) with 19% proton incorporation alpha to the ketone group.

Potassium carbonate (0.66 g, 4.77 mmol) was added to a solution of thus obtained 4d (1.16 g, 3.18 mmol) in a 1:1 mixture of deuterium oxide (100 mL, CIL, 99.9 atom % D) and anhydrous THE (100 mL) at rt. The reaction mixture was stirred for 3d. The reaction mixture was partitioned between saturated brine in deuterium oxide (60 mL, CIL, 99.9 atom % D) and CH₂Cl₂ (300 mL). The layers were separated and the aqueous layer was extracted with CH₂Cl₂ (200 mL). The combined organic layers were washed with saturated brine in deuterium oxide (100 mL, CIL, 99.9 atom % D), dried (Na₂SO₄), filtered and concentrated under reduced pressure to give 4d (1.18 g) as a clear oil.

Step 2. (2,3-Bis(methoxy-d₃)phenyl)(piperidin-4-yl-2,2,3,3,4,5,5,6,6-d₉)methanone trifluoroacetate salt (5d). Trifluoroacetic acid-d (10.88 g, 95.5 mmol, Sigma Aldrich, 99.5 atom % D) was added neat at 0° C. to 4d (1.16 g, 3.2 mmol). The reaction mixture was warmed to room temperature and stirred for 3 h. The reaction mixture was concentrated under reduced pressure to give a yellow oil. Et₂O (100 mL) was added to the residue. The mixture was stirred for 10 minutes giving a heavy white precipitate. The solid was filtered to give 5d (1.17 g, quantitative yield) as a white solid.

Step 3. (2,3-Bis(methoxy-d₃)phenyl)(piperidin-4-yl-2,2,3,3,4,5,5,6,6-d₉)methanol (30c). Sodium borohydride (0.49 g, 12.9 mmol) was added in one portion to a solution of 5d (1.17 g, 3.22 mmol) in MeOD (60 mL, Sigma Aldrich, 99.5 atom % D) at 0° C. The reaction mixture was warmed to room temperature and stirred overnight. The reaction mixture was concentrated under reduced pressure and the residue was partitioned between saturated sodium bicarbonate solution in deuterium oxide (50 mL, CIL, 99.9 atom % D) and CH₂Cl₂ (100 mL). The layers were separated and the aqueous layer was extracted with CH₂Cl₂ (2×100 mL). The combined organic layers were washed with deuterium oxide (50 mL, CIL, 99.9 atom % D), dried (Na₂SO₄), filtered and concentrated under reduced pressure to give 30c (0.26 g). The combined aqueous layer was concentrated under reduced pressure and the residue was extracted with CH₂Cl₂ (3×100 mL). The combined organic extracts were concentrated under reduced pressure to give 30c (0.74 g, 87%).

Step 4. (2,3-Bis(methoxy-d₃)phenyl)(1-(4-fluorophenethyl)piperidin-4-yl-2,2,3,3,4,5,5,6,6-d₉)methanol (7k). Sodium bicarbonate powder (0.15 g, 1.8 mmol) followed by a solution of 9a (0.18 g, 0.90 mmol) in DMF (3 mL) were added to a solution of 30c (0.24 g, 0.90 mmol) in DMF (7 mL) at room temperature. The reaction mixture was heated at 90° C. for 2.5 h, cooled to rt, and concentrated under reduced pressure. The residue was diluted with water (10 mL) and extracted with EtOAc (3×25 mL). The combined organic layers were washed with saturated brine (20 mL), dried (Na₂SO₄), filtered, and concentrated under reduced pressure to give crude 7k (0.31 g, 88%) as a clear oil, which became solid upon standing.

Chiral separation. (R)-(2,3-bis(methoxy-d₃)phenyl)(1-(4-fluorophenethyl)piperidin-4-yl-2,2,3,3,4,5,5,6,6-d₉)methanol (Compound 347). 7k (290 mg) was purified by chiral SFC (AD-H, 250×20 mm, 5 mm; 20% EtOH (0.1% diethylamine/CO₂, 100 bar; flow rate 60 mL/min; wavelength 220 nm), to yield the early eluting (R) enantiomer (154 mg) as a clear oil.

The resulting clear oil was triturated with CH₂Cl₂ and hexane to give Compound 347 (102 mg) as a white solid.

¹H NMR (400 MHz, CDCL₃) δ 2.32 (br s, 1H), 2.51 (d, J=16.5 Hz, 2H), 2.75 (d, J=16.5 Hz, 2H), 4.63 (s, 1H), 6.84 (d, J=6.9 Hz, 1H), 6.89 (d, J=7.5 Hz, 1H), 6.95 (t, J=8.7 Hz, 2H), 7.04 (t, J=7.9 Hz, 1H), 7.10-7.18 (m, 2H).

LCMS (method: Atlantis T3 column, 2.1×50 mm, 3 μm; 5-95% acetonitrile/water with 0.1% formic acid in 14 min, with 4 min hold; flow rate: 0.7 mL/min; wavelength: 210 nm): retention time 4.6 min; 99.8% purity; (EI-MS): m/z=389.3 ([M+H]⁺)

Chiral HPLC (Chiralpak IG column, 250 mm×4.6 mm, 5 μm, method: 100% EtOH; flow rate: 0.3 mL/min; wavelength: 230 nm): retention times: (R) isomer: 16.9 min, (S) isomer: 21.4 min; 99.3% ee.

### Example 10. Synthesis of (R)-(2,3-bis(methoxy-d₃)phenyl)(1-(2-(4-fluorophenyl)ethyl-1,1-d₂)piperidin-4-yl-2,2,3,3,4,5,5,6,6-d₉)methanol (Compound 359)

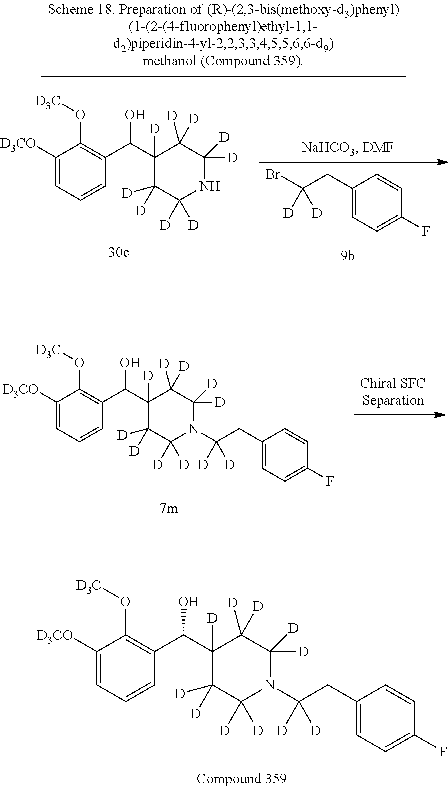

Step 1. (2,3-Bis(methoxy-d₃)phenyl)(1-(2-(4-fluorophenyl)ethyl-1,1-d₂)piperidin-4-yl-2,2,3,3,4,5,5,6,6-d₉)methanol (7m). Sodium bicarbonate powder (0.15 g, 1.8 mmol) followed by a solution of 9b (0.19 g, 0.90 mmol) in DMF (3 mL) were added to a solution of 30c (0.24 g, 0.90 mmol) in DMF (7 mL) at room temperature. The reaction mixture was heated at 90° C. for 2.5 h, cooled to room temperature and concentrated under reduced pressure. The residue was diluted with water (10 mL) and extracted with EtOAc (3×25 mL). The combined organic layers were washed with saturated brine (20 mL), dried (Na₂SO₄), filtered and concentrated under reduced pressure to give 7m (0.30 g, 83%) as a clear oil which solidified on standing.

Chiral separation of (R)-(2,3-bis(methoxy-d₃)phenyl)(1-(2-(4-fluorophenyl)ethyl-1,1-d₂)piperidin-4-yl-2,2,3,3,4,5,5,6,6-d₉)methanol (Compound 359). 7m (289 mg) was purified by chiral SFC (AD-H, 250×20 mm, 5 mm; 20% EtOH (0.1% diethylamine/CO₂, 100 Bar; flow rate 60 mL/min; wavelength 220 nm), to yield the early eluting (R) enantiomer as a clear oil (148 mg). The resulting clear oil was triturated with CH₂Cl₂ and hexane to give Compound 359 (113 mg) as a white solid.

¹H NMR (400 MHz, CDCl₃) δ 2.32 (br s, 1H), 2.74 (s, 2H), 4.63 (s, 1H), 6.84 (d, J=8.1 Hz, 1H), 6.89 (d, J=7.8 Hz, 1H), 6.95 (t, J=8.7 Hz, 2H), 7.04 (t, J=7.9 Hz, 1H), 7.13 (d, J=13.9 Hz, 2H).

LCMS (method: Atlantis T3 column, 2.1×50 mm, 3 μm; 5-95% acetonitrile/water with 0.1% formic acid in 14 min, with 4 min hold; flow rate: 0.7 mL/min; wavelength: 210 nm): 4.5 min; 99.8% purity; (ET-MS): m/z=391.3 ([M+H]⁺)

Chiral HPLC (Chiralpak IG column, 250 mm×4.6 mm, 3 μm, method: 100% EtOH; flow rate: 0.3 mL/min; wavelength: 230 nm): retention times: (R) isomer: 16.8 min, (S) isomer: 21.4 min; 99.2% ee.

### Example 11. Synthesis of (R)-(2,3-bis(methoxy-d₃)phenyl)(1-(2-(4-fluorophenyl)ethyl-1,1,2,22-d₄)piperidin-4-yl-2,2,3,3,4,5,5,6,6-d₉)methanol (Compound 383)

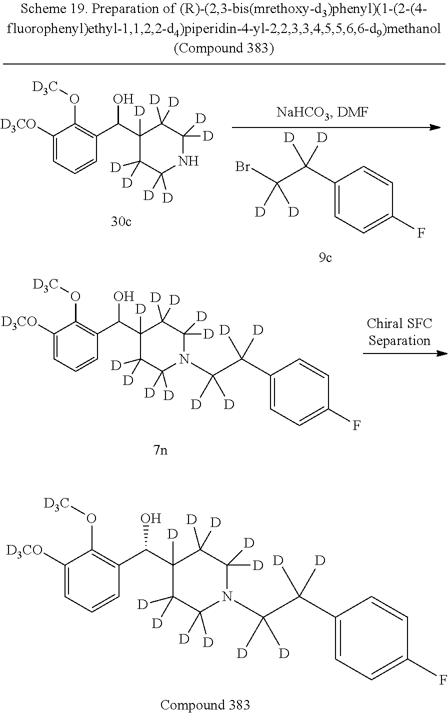

Step 1. (2,3-Bis(methoxy-d₃)phenyl)(1-(2-(4-fluorophenyl)ethyl-1,1,2,2-d₄)piperidin-4-yl-2,2,3,3,4,5,5,6,6-d₉)methanol (7n). To a solution of 30c (0.27 g, 1.0 mmol) in DMF (7 mL) was added NaHCO₃ (0.17 g, 2.0 mmol), followed by a solution of 9c (0.21 g, 1.0 mmol) in DMF (3 mL). The reaction mixture was heated at 90° C. for 2.5 h, cooled to room temperature and concentrated under reduced pressure. The residue was diluted with water (10 mL) and extracted with EtOAc (3×25 mL). The combined organic layers were washed with saturated brine (20 mL), dried (Na₂SO₄), filtered and concentrated under reduced pressure to give 7n (0.35 g, 90%) as a clear oil which solidified on standing.

Chiral separation of (R)-(2,3-bis(methoxy-d₃)phenyl)(1-(2-(4-fluorophenyl)ethyl-1,1,2,2-d₄)piperidin-4-yl-2,2,3,3,4,5,5,6,6-d₉)methanol (Compound 383). 7n (350 mg) was purified by chiral SFC (AD-H, 250×20 mm, 5 mm; 20% EtOH (0.1% diethylamine/CO₂, 100 Bar; flow rate 60 mL/min; wavelength 220 nm), to yield the early eluting (R) enantiomer as a clear oil (150 mg).

The resulting clear oil was triturated with CH₂Cl₂ and hexane to give Compound 359 (142 mg) as a white solid.

¹H NMR (400 MHz, CDCl₃) δ 2.32 (s, 1H), 4.63 (s, 1H), 6.84 (d, J=8.1 Hz, 1H), 6.89 (d, J=7.8 Hz, 1H), 6.95 (t, J=8.8 Hz, 2H), 7.04 (t, J=7.9 Hz, 1H), 7.13 (d, J=14.1 Hz, 2H).

LCMS (method: Atlantis T3 column, 2.1×50 mm, 3 μm; 5-95% acetonitrile/water with 0.1% formic acid in 14 min, with 4 min hold; flow rate: 0.7 mL/min; wavelength: 210 nm): retention time: 4.5 min; 99.9% purity; (EI-MS): m/z=393.3 ([M+H]⁺)

Chiral HPLC (Chiralpak IG column, 250 mm×4.6 mm, 5 μm, method: 100% EtOH; flow rate: 0.3 mL/min; wavelength: 230 nm): retention times: (R) isomer: 16.8 min, (S) isomer: 21.4 min; 99.3% ee.

### Example 12. Evaluation of Metabolic Stability in Human Liver Microsomes

Microsomal Assay: Human liver microsomes (20 mg/mL) were obtained from Xenotech, LLC (Lenexa, KS). β-nicotinamide adenine dinucleotide phosphate, reduced form (NADPH), magnesium chloride (MgCl₂), and dimethyl sulfoxide (DMSO) were purchased from Sigma-Aldrich.

Determination of Metabolic Stability: 7.5 mM stock solutions of test compounds of structural formula (I) (e.g., of an embodiment or aspect of embodiment thereof described herein) or structural formula (II), or pharmaceutically acceptable salt thereof, were prepared in DMSO. The 7.5 mM stock solutions were diluted to 12.5-50 μM in acetonitrile (ACN). The 20 mg/mL human liver microsomes were diluted to 0.625 mg/mL in 0.1 M potassium phosphate buffer, pH 7.4, containing 3 mM MgCl₂. The diluted microsomes were added to wells of a 96-well deep-well polypropylene plate in triplicate. A 10 μL aliquot of the 12.5-50 μM test compound was added to the microsomes and the mixture was pre-warmed for 10 minutes. Reactions were initiated by addition of pre-warmed NADPH solution. The final reaction volume was 0.5 mL and contained 4.0 mg/mL human liver microsomes, 0.25 μM test compound, and 2 mM NADPH in 0.1 M potassium phosphate buffer, pH 7.4, and 3 mM MgCl₂. The reaction mixtures were incubated at 37° C., and 50 μL aliquots were removed at 0, 5, 10, 20, and 30 minutes and added to shallow-well 96-well plates which contain 50 μL of ice-cold ACN (acetonitrile) with internal standard to stop the reactions. The plates were stored at 4° C. for 20 minutes after which 100 μL of water was added to the wells of the plate before centrifugation to pellet precipitated proteins. Supernatants were transferred to another 96-well plate and analyzed for amounts of parent remaining by LC-MS/MS using an Applied Bio-systems API 4000 mass spectrometer. The same procedure was followed for the non-deuterated counterpart of the compound of Formula I and the positive control, 7-ethoxycoumarin (1 μM). Testing was done in triplicate.

Data analysis: The in vitro t_{1/2}s for test compounds were calculated from the slopes of the linear regression of % parent remaining (ln) vs incubation time relationship.

$$in vitro {t}_{1 / 2} = 0.693 / k$$

$$k = - [ \text{} slope of linear regression of \% parent remaining ( ln ) vs incubation time ]$$

The apparent intrinsic clearance was calculated using the following equation:

$${CL}_{int} ( mL / min / kg ) = ( 0.693 / in vitro {t}_{1 / 2} ) ( Incubation Volume / mg of microsomes ) ( 45 mg microsomes / gram of liver ) ( 20 gm of liver / kg b . w . )$$

Data analysis was performed using Microsoft Excel Software. Results are shown in Tables 5 and 6 below.

TABLE 5

| | EXP# 2506 | EXP# 2506 | |
|---|---|---|---|
| | t_{1/2} | CLᵢₙₜ | |
| Compound ID | (min) | (mL/min/kg) | % Δ |
| volinanserin | 48.5 | 3.2 | |
| Compound 147 | 67.2 | 2.3 | 38.7 |
| Compound 115 | 69.5 | 2.3 | 43.3 |
| Compound 148 | 71.2 | 2.2 | 46.9 |
| Compound 131 | 71.3 | 2.4 | 47.1 |
| Compound 151 | 69.9 | 2.3 | 44.3 |
| Compound 159 | 84.9 | 1.9 | 75.0 |

TABLE 6

| | EXP# 2503 | EXP#2503 | |
|---|---|---|---|
| | t_{1/2} | CLᵢₙₜ | |
| Compound ID | (min) | (mL/min/kg) | % Δ |
| volinanserin | 44.69 | 3.5 | |
| Compound 203 | 47.65 | 3.4 | 6.6 |
| Compound 215 | 46.62 | 3.4 | 4.3 |
| Compound 347 | 48.92 | 3.2 | 9.5 |
| Compound 359 | 65.80 | 2.4 | 47.2 |
| Compound 383 | 74.04 | 2.3 | 65.7 |

In these experiments, values equal to or more than a 15% increase in half-life are considered to be a significant difference. If the apparent intrinsic clearance ratio (deuterated compound/volinanserin) is >1.15 or ≤0.85, then there is considered to be significant differentiation.

The results show that deuterated Compounds 147, 115, 148, 131, 151, 159, 359, and 383 display a significant increase in half-life (t_{1/2}) in human liver microsomes as compared to undeuterated volinanserin, whereas deuterated Compounds 203, 215, and 347 did not.

### Example 13. Evaluation of Metabolic Stability in CYP3A4 Supersomes

### Materials and Methods:

Materials: CYP3A4 supersomes™ were obtained from Corning Gentest. β-nicotinamide adenine dinucleotide phosphate, reduced form (NADPH), magnesium chloride (MgCl₂), and dimethyl sulfoxide (DMSO) were purchased from Sigma-Aldrich.D-Crizotinib compounds were supplied by Concert Pharmaceuticals.

Determination of Metabolic Stability: 10 mM stock solutions of test compounds were prepared in DMSO. The 7.5 mM stock solutions were diluted to 12.75 μM in acetonitrile (ACN). The CYP3A4 supersomes were diluted to 50 pmol/mL in 0.1 M potassium phosphate buffer, pH 7.4, containing 3 mM MgCl₂. The diluted supersomes were added to wells of a 96-well deep-well polypropylene plate in triplicate. 10 μL of the 12.75 μM test compound was added to the supersomes and the mixture was pre-warmed for 10 minutes. Reactions were initiated by addition of pre-warmed NADPH solution. The final reaction volume was 0.5 mL and contained 50 pmol/mL CYP3A4 supersomes, 0.25 μM test compound, and 2 mM NADPH in 0.1 M potassium phosphate buffer, pH 7.4, and 3 mM MgCl₂. The reaction mixtures were incubated at 37° C. and 50 μL aliquots were removed at 0, 5, 10, 20, and 30 minutes and added to shallow-well 96-well plates which contained 50 μL of ice-cold ACN with internal standard to stop the reactions. The plates were stored at 4° C. for 20 minutes after which 100 μL of water was added to the wells of the plate before centrifugation to pellet precipitated proteins. Supernatants were transferred to another 96-well plate and analyzed for amounts of parent remaining by LC-MS/MS using an Applied Bio-systems API 4000 mass spectrometer.

Data analysis: The in vitro t_{1/2}s for test compounds were calculated from the slopes of the linear regression of % parent remaining (ln) vs incubation time relationship.

$$in vitro {t}_{1 / 2} = 0.693 / k$$

$$k = - [ \text{} slope of linear regression of \% parent remaining ( ln ) vs incubation time ]$$

Data analysis was performed using Microsoft Excel Software. Results are shown in Tables 7 and 8 below.

TABLE 7

| | EXP# 2503 | |
|---|---|---|
| | t_{1/2} | |
| Compound ID | (min) | % Δ |
| volinanserin | 12.6 | |
| Compound 147 | 20.4 | 61.4 |
| Compound 115 | 19.1 | 50.9 |
| Compound 148 | 19.6 | 54.9 |
| Compound 131 | 17.5 | 38.5 |
| Compound 151 | 16.8 | 33.0 |
| Compound 159 | 18.1 | 43.2 |

TABLE 8

| | EXP# 2505 | |
|---|---|---|
| | t_{1/2} | |
| Compound ID | (min) | % Δ |
| volinanserin | 14.6 | |
| Compound 203 | 13.7 | −5.9 |
| Compound 215 | 15.0 | 2.9 |
| Compound 347 | 20.4 | 39.7 |
| Compound 359 | 19.3 | 32.3 |
| Compound 383 | 19.0 | 30.1 |

In these experiments, values equal to or more than a 15% increase in half-life are considered to be a significant difference. The results show that deuterated Compounds 147, 115, 148, 131, 151, 159, 347, 359, and 383 display a significant increase in half-life (t_{1/2}) in CYP3A4 supersomes as compared to undeuterated volinanserin, whereas deuterated Compounds 203 and 215 did not.

The relevant teachings of all patents, published applications and references cited herein are incorporated by reference in their entirety.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

What is claimed is:

1. A compound represented by structural formula (I): 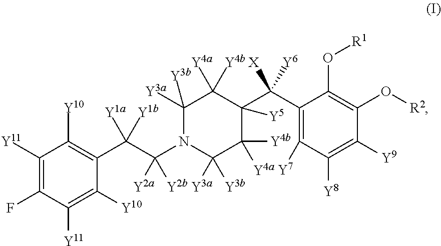
or a pharmaceutically acceptable salt thereof, wherein:
R¹ and R² are independently selected from —CH₃, —CH₂D, —CHD₂, and —CD₃;
X is —OH, or —F; and
Y^{1a}, Y^{1b}, Y^{2a}, Y^{2b}, Y^{3a}, Y^{3b}, Y^{4a}, Y^{4b}, Y⁵, Y⁶, Y⁷, Y⁸, Y⁹, Y¹⁰, and Y¹¹ are each independently selected from hydrogen and deuterium;
provided that at least one of Y^{1a}, Y^{1b}, Y^{2a}, Y^{2b}, Y^{3a}, Y^{3b}, Y^{4a}, Y^{4b}, Y⁵, Y⁶, Y⁷, Y⁸, Y⁹, Y¹⁰, Y¹¹, R¹ and R² comprises deuterium;
provided that when Y^{1a}, Y^{1b}, Y^{2a}, and Y^{2b} are each deuterium, then at least one of Y^{3a}, Y^{3b}, Y^{4a}, Y^{4b}, Y⁵, Y⁶, Y⁷, Y⁸, Y⁹, Y¹⁰, Y¹¹, R¹ and R² comprises deuterium; and
provided that when Y^{3a}, Y^{3b}, Y^{4a}, and Y^{4b} are each deuterium, then at least one of Y^{1a}, Y^{1b}, Y^{2a}, Y^{2b}, Y⁵, Y⁶, Y⁷, Y⁸, Y⁹, Y¹⁰, Y¹¹, R¹ and R² comprises deuterium;
wherein each position designated specifically as deuterium has at least 50.1% incorporation of deuterium.

2. The compound of claim 1, wherein R¹ and R² are independently selected from —CH₃ and —CD₃.

3. The compound of any one of the preceding claims, wherein X is —OH.

4. The compound of any one of the preceding claims, wherein Y⁷, Y⁸, Y⁹, Y¹⁰, and Y¹¹ are each hydrogen.

5. The compound of any one of the preceding claims, wherein Y^{4a} and Y⁴ are the same.

6. The compound of any one of the preceding claims, wherein Y^{4a}, Y^{4b}, Y⁵, Y⁷, Y⁸, Y⁹, Y¹⁰, and Y¹¹ are each hydrogen.

7. The compound of any one of the preceding claims, wherein Y^{1a} and Y^{1b} are the same.

8. The compound of any one of the preceding claims, wherein Y^{2a} and Y^{2b} are the same.

9. The compound of any one of the preceding claims, wherein Y^{3a} and Y^{3b} are the same.

10. The compound of any one of the preceding claims, wherein R¹ and R² are independently selected from —CH₃ and —CD₃; X is —OH; Y^{4a}, Y^{4b}, Y⁵, Y⁷, Y⁸, Y⁹, Y¹⁰, and Y¹¹ are each hydrogen; Y^{1a}, Y^{1b}, Y^{2a} and Y^{2b} are the same; and Y^{3a} and Y^{3b} are the same.

11. The compound of any one of the preceding claims, wherein R¹ and R² are independently selected from —CH₃ and —CD₃; X is —OH; Y^{4a}, Y^{4b}, Y⁵, Y⁷, Y⁸, Y⁹, Y¹⁰, and Y¹¹ are each hydrogen; Y^{1a} and Y^{1b} are the same; and Y^{2a}, Y^{2b}, Y^{3a} and Y^{3b} are the same.

12. The compound of any one of the preceding claims, wherein Y^{2a} and Y² are deuterium.

13. The compound of any one of the preceding claims, wherein any atom not designated as deuterium is present at its natural isotopic abundance.

14. The compound of claim 1, wherein X is —OH; Y^{1a} and Y^{1b} are the same; Y^{2a} and Y^{2b} are the same; Y^{3a} and Y^{3b} are the same; and Y^{4a}, Y^{4b}, Y⁵, Y⁷, Y⁸, Y⁹, Y¹⁰, and Y¹¹ are each hydrogen; and the compound is selected from any one of the Compounds set forth in Table 1 below:
TABLE 1

| Compound # | R¹ | R² | Y^{1a}/Y^{1b} | Y^{2a}/Y^{2b} | Y^{3a}/Y^{3b} | Y⁶ |
|---|---|---|---|---|---|---|
| 100 | CH₃ | CH₃ | H | H | H | D |
| 101 | CH₃ | CH₃ | H | H | D | H |
| 102 | CH₃ | CH₃ | H | H | D | D |
| 103 | CH₃ | CH₃ | H | D | H | H |
| 104 | CH₃ | CH₃ | H | D | H | D |
| 105 | CH₃ | CH₃ | H | D | D | H |
| 106 | CH₃ | CH₃ | H | D | D | D |
| 107 | CH₃ | CH₃ | D | H | H | H |
| 108 | CH₃ | CH₃ | D | H | H | D |
| 109 | CH₃ | CH₃ | D | H | D | H |
| 110 | CH₃ | CH₃ | D | H | D | D |
| 112 | CH₃ | CH₃ | D | D | H | D |
| 113 | CH₃ | CH₃ | D | D | D | H |
| 114 | CH₃ | CH₃ | D | D | D | D |
| 115 | CH₃ | CD₃ | H | H | H | H |
| 116 | CH₃ | CD₃ | H | H | H | D |
| 117 | CH₃ | CD₃ | H | H | D | H |
| 118 | CH₃ | CD₃ | H | H | D | D |
| 119 | CH₃ | CD₃ | H | D | H | H |
| 120 | CH₃ | CD₃ | H | D | H | D |
| 121 | CH₃ | CD₃ | H | D | D | H |
| 122 | CH₃ | CD₃ | H | D | D | D |
| 123 | CH₃ | CD₃ | D | H | H | H |
| 124 | CH₃ | CD₃ | D | H | H | D |
| 125 | CH₃ | CD₃ | D | H | D | H |
| 126 | CH₃ | CD₃ | D | H | D | D |
| 127 | CH₃ | CD₃ | D | D | H | H |
| 128 | CH₃ | CD₃ | D | D | H | D |
| 129 | CH₃ | CD₃ | D | D | D | H |
| 130 | CH₃ | CD₃ | D | D | D | D |
| 131 | CD₃ | CH₃ | H | H | H | H |
| 132 | CD₃ | CH₃ | H | H | H | D |
| 133 | CD₃ | CH₃ | H | H | D | H |
| 134 | CD₃ | CH₃ | H | H | D | D |
| 135 | CD₃ | CH₃ | H | D | H | H |
| 136 | CD₃ | CH₃ | H | D | H | D |
| 137 | CD₃ | CH₃ | H | D | D | H |
| 138 | CD₃ | CH₃ | H | D | D | D |
| 139 | CD₃ | CH₃ | D | H | H | H |
| 140 | CD₃ | CH₃ | D | H | H | D |
| 141 | CD₃ | CH₃ | D | H | D | H |
| 142 | CD₃ | CH₃ | D | H | D | D |
| 143 | CD₃ | CH₃ | D | D | H | H |
| 144 | CD₃ | CH₃ | D | D | H | D |
| 145 | CD₃ | CH₃ | D | D | D | H |
| 146 | CD₃ | CH₃ | D | D | D | D |
| 147 | CD₃ | CD₃ | H | H | H | H |
| 148 | CD₃ | CD₃ | H | H | H | D |
| 149 | CD₃ | CD₃ | H | H | D | H |
| 150 | CD₃ | CD₃ | H | H | D | D |
| 151 | CD₃ | CD₃ | H | D | H | H |
| 152 | CD₃ | CD₃ | H | D | H | D |
| 153 | CD₃ | CD₃ | H | D | D | H |
| 154 | CD₃ | CD₃ | H | D | D | D |
| 155 | CD₃ | CD₃ | D | H | H | H |
| 156 | CD₃ | CD₃ | D | H | H | D |
| 157 | CD₃ | CD₃ | D | H | D | H |
| 158 | CD₃ | CD₃ | D | H | D | D |
| 159 | CD₃ | CD₃ | D | D | H | H |
| 160 | CD₃ | CD₃ | D | D | H | D |
| 161 | CD₃ | CD₃ | D | D | D | H |
| 162 | CD₃ | CD₃ | D | D | D | D |

or a pharmaceutically acceptable salt of any of the foregoing, wherein any atom not designated as deuterium is present at its natural isotopic abundance.

15. The compound of claim 1, wherein X is —OH; Y^{1a} and Y^{1b} are the same; Y^{2a} and Y^{2b} are the same; Y^{3a} and Y^{3b} are the same; and Y^{4a}, Y^{4b}, Y⁵, Y⁷, Y⁸, Y⁹, Y¹⁰ and Y¹¹ are each hydrogen; and the compound is selected from any one of the Compounds set forth in Table 2 below:
TABLE 2

| Compound # | R¹ | R² | Y^{1a}/Y^{1b} | Y^{2a}/Y^{2b} | Y^{3a}/Y^{3b} | Y⁶ |
|---|---|---|---|---|---|---|
| 131 | CD₃ | CH₃ | H | H | H | H |
| 115 | CH₃ | CD₃ | H | H | H | H |
| 147 | CD₃ | CD₃ | H | H | H | H |
| 132 | CD₃ | CH₃ | H | H | H | D |
| 116 | CH₃ | CD₃ | H | H | H | D |
| 148 | CD₃ | CD₃ | H | H | H | D |
| 139 | CD₃ | CH₃ | D | H | H | H |
| 123 | CH₃ | CD₃ | D | H | H | H |
| 155 | CD₃ | CD₃ | D | H | H | H |
| 135 | CD₃ | CH₃ | H | D | H | H |
| 119 | CH₃ | CD₃ | H | D | H | H |
| 151 | CD₃ | CD₃ | H | D | H | H |
| 137 | CD₃ | CH₃ | H | D | D | H |
| 121 | CH₃ | CD₃ | H | D | D | H |
| 153 | CD₃ | CD₃ | H | D | D | H |
| 138 | CD₃ | CH₃ | H | D | D | D |
| 122 | CH₃ | CD₃ | H | D | D | D |
| 154 | CD₃ | CD₃ | H | D | D | D |
| 145 | CD₃ | CH₃ | D | D | D | H |
| 129 | CH₃ | CD₃ | D | D | D | H |
| 161 | CD₃ | CD₃ | D | D | D | H |
| 146 | CD₃ | CH₃ | D | D | D | D |
| 130 | CH₃ | CD₃ | D | D | D | D |
| 162 | CD₃ | CD₃ | D | D | D | D |

or a pharmaceutically acceptable salt of any of the foregoing, wherein any atom not designated as deuterium is present at its natural isotopic abundance.

16. The compound of claim 1, wherein X is —OH; Y^{1a} and Y^{1b} are the same; Y^{2a} and Y^{2b} are the same; Y^{3a} and Y^{3b} are the same; Y^{4a} and Y^{4b} are the same; and Y⁷, Y⁸, Y⁹, Y¹⁰, and Y¹¹ are each hydrogen; and the compound is selected from any one of the Compounds set forth in Table 3
TABLE 3

| Compound # | R¹ | R² | Y^{1a}/Y^{1b} | Y^{2a}/Y^{2b} | Y^{3a}/Y^{3b} | Y^{4a}/Y^{4b} | Y⁵ | Y⁶ |
|---|---|---|---|---|---|---|---|---|
| 200 | CH₃ | CH₃ | H | H | H | D | D | D |
| 201 | CH₃ | CH₃ | H | H | H | D | H | D |
| 202 | CH₃ | CH₃ | H | H | H | H | D | D |
| 203 | CH₃ | CH₃ | H | H | D | D | D | H |
| 204 | CH₃ | CH₃ | H | H | D | D | H | H |
| 205 | CH₃ | CH₃ | H | H | D | H | D | H |
| 206 | CH₃ | CH₃ | H | H | D | D | D | D |
| 207 | CH₃ | CH₃ | H | H | D | D | H | D |
| 208 | CH₃ | CH₃ | H | H | D | H | D | D |
| 209 | CH₃ | CH₃ | H | D | H | D | D | H |
| 210 | CH₃ | CH₃ | H | D | H | D | H | H |
| 211 | CH₃ | CH₃ | H | D | H | H | D | H |
| 212 | CH₃ | CH₃ | H | D | H | D | D | D |
| 213 | CH₃ | CH₃ | H | D | H | D | H | D |
| 214 | CH₃ | CH₃ | H | D | H | H | D | D |
| 215 | CH₃ | CH₃ | H | D | D | D | D | H |
| 216 | CH₃ | CH₃ | H | D | D | D | H | H |
| 217 | CH₃ | CH₃ | H | D | D | H | D | H |
| 218 | CH₃ | CH₃ | H | D | D | D | D | D |
| 219 | CH₃ | CH₃ | H | D | D | D | H | D |
| 220 | CH₃ | CH₃ | H | D | D | H | D | D |
| 221 | CH₃ | CH₃ | D | H | H | D | D | H |
| 222 | CH₃ | CH₃ | D | H | H | D | H | H |
| 223 | CH₃ | CH₃ | D | H | H | H | D | H |
| 224 | CH₃ | CH₃ | D | H | H | D | D | D |
| 225 | CH₃ | CH₃ | D | H | H | D | H | D |
| 226 | CH₃ | CH₃ | D | H | H | H | D | D |
| 227 | CH₃ | CH₃ | D | H | D | D | D | H |
| 228 | CH₃ | CH₃ | D | H | D | D | H | H |
| 229 | CH₃ | CH₃ | D | H | D | H | D | H |
| 230 | CH₃ | CH₃ | D | H | D | D | D | D |
| 231 | CH₃ | CH₃ | D | H | D | D | H | D |
| 232 | CH₃ | CH₃ | D | H | D | H | D | D |
| 233 | CH₃ | CH₃ | D | D | H | D | D | H |
| 234 | CH₃ | CH₃ | D | D | H | D | H | H |
| 235 | CH₃ | CH₃ | D | D | H | H | D | H |
| 236 | CH₃ | CH₃ | D | D | H | D | D | D |
| 237 | CH₃ | CH₃ | D | D | H | D | H | D |
| 238 | CH₃ | CH₃ | D | D | H | H | D | D |
| 239 | CH₃ | CH₃ | D | D | D | D | D | H |
| 240 | CH₃ | CH₃ | D | D | D | D | H | H |
| 241 | CH₃ | CH₃ | D | D | D | H | D | H |
| 242 | CH₃ | CH₃ | D | D | D | D | D | D |
| 243 | CH₃ | CH₃ | D | D | D | D | H | D |
| 244 | CH₃ | CH₃ | D | D | D | H | D | D |
| 245 | CH₃ | CD₃ | H | H | H | D | D | H |
| 246 | CH₃ | CD₃ | H | H | H | D | H | H |
| 247 | CH₃ | CD₃ | H | H | H | H | D | H |
| 248 | CH₃ | CD₃ | H | H | H | D | D | D |
| 249 | CH₃ | CD₃ | H | H | H | D | H | D |
| 250 | CH₃ | CD₃ | H | H | H | H | D | D |
| 251 | CH₃ | CD₃ | H | H | D | D | D | H |
| 252 | CH₃ | CD₃ | H | H | D | D | H | H |
| 253 | CH₃ | CD₃ | H | H | D | H | D | H |
| 254 | CH₃ | CD₃ | H | H | D | D | D | D |
| 255 | CH₃ | CD₃ | H | H | D | D | H | D |
| 256 | CH₃ | CD₃ | H | H | D | H | D | D |
| 257 | CH₃ | CD₃ | H | D | H | D | D | H |
| 258 | CH₃ | CD₃ | H | D | H | D | H | H |
| 259 | CH₃ | CD₃ | H | D | H | H | D | H |
| 260 | CH₃ | CD₃ | H | D | H | D | D | D |
| 261 | CH₃ | CD₃ | H | D | H | D | H | D |
| 262 | CH₃ | CD₃ | H | D | H | H | D | D |
| 263 | CH₃ | CD₃ | H | D | D | D | D | H |
| 264 | CH₃ | CD₃ | H | D | D | D | H | H |
| 265 | CH₃ | CD₃ | H | D | D | H | D | H |
| 266 | CH₃ | CD₃ | H | D | D | D | D | D |
| 267 | CH₃ | CD₃ | H | D | D | D | H | D |
| 268 | CH₃ | CD₃ | H | D | D | H | D | D |
| 269 | CH₃ | CD₃ | D | H | H | D | D | H |
| 270 | CH₃ | CD₃ | D | H | H | D | H | H |
| 271 | CH₃ | CD₃ | D | H | H | H | D | H |
| 272 | CH₃ | CD₃ | D | H | H | D | D | D |
| 273 | CH₃ | CD₃ | D | H | H | D | H | D |
| 274 | CH₃ | CD₃ | D | H | H | H | D | D |
| 275 | CH₃ | CD₃ | D | H | D | D | D | H |
| 276 | CH₃ | CD₃ | D | H | D | D | H | H |
| 277 | CH₃ | CD₃ | D | H | D | H | D | H |
| 278 | CH₃ | CD₃ | D | H | D | D | D | D |
| 279 | CH₃ | CD₃ | D | H | D | D | H | D |
| 280 | CH₃ | CD₃ | D | H | D | H | D | D |
| 281 | CH₃ | CD₃ | D | D | H | D | D | H |
| 282 | CH₃ | CD₃ | D | D | H | D | H | H |
| 283 | CH₃ | CD₃ | D | D | H | H | D | H |
| 284 | CH₃ | CD₃ | D | D | H | D | D | D |
| 285 | CH₃ | CD₃ | D | D | H | D | H | D |
| 286 | CH₃ | CD₃ | D | D | H | H | D | D |
| 287 | CH₃ | CD₃ | D | D | D | D | D | H |
| 288 | CH₃ | CD₃ | D | D | D | D | H | H |
| 289 | CH₃ | CD₃ | D | D | D | H | D | H |
| 290 | CH₃ | CD₃ | D | D | D | D | D | D |
| 291 | CH₃ | CD₃ | D | D | D | D | H | D |
| 292 | CH₃ | CD₃ | D | D | D | H | D | D |
| 293 | CD₃ | CH₃ | H | H | H | D | D | H |
| 294 | CD₃ | CH₃ | H | H | H | D | H | H |
| 295 | CD₃ | CH₃ | H | H | H | H | D | H |
| 296 | CD₃ | CH₃ | H | H | H | D | D | D |
| 297 | CD₃ | CH₃ | H | H | H | D | H | D |
| 298 | CD₃ | CH₃ | H | H | H | H | D | D |
| 299 | CD₃ | CH₃ | H | H | D | D | D | H |
| 300 | CD₃ | CH₃ | H | H | D | D | H | H |
| 301 | CD₃ | CH₃ | H | H | D | H | D | H |
| 302 | CD₃ | CH₃ | H | H | D | D | D | D |
| 303 | CD₃ | CH₃ | H | H | D | D | H | D |
| 304 | CD₃ | CH₃ | H | H | D | H | D | D |
| 305 | CD₃ | CH₃ | H | D | H | D | D | H |
| 306 | CD₃ | CH₃ | H | D | H | D | H | H |
| 307 | CD₃ | CH₃ | H | D | H | H | D | H |
| 308 | CD₃ | CH₃ | H | D | H | D | D | D |
| 309 | CD₃ | CH₃ | H | D | H | D | H | D |
| 310 | CD₃ | CH₃ | H | D | H | H | D | D |
| 311 | CD₃ | CH₃ | H | D | D | D | D | H |
| 312 | CD₃ | CH₃ | H | D | D | D | H | H |
| 313 | CD₃ | CH₃ | H | D | D | H | D | H |
| 314 | CD₃ | CH₃ | H | D | D | D | D | D |
| 315 | CD₃ | CH₃ | H | D | D | D | H | D |
| 316 | CD₃ | CH₃ | H | D | D | H | D | D |
| 317 | CD₃ | CH₃ | D | H | H | D | D | H |
| 318 | CD₃ | CH₃ | D | H | H | D | H | H |
| 319 | CD₃ | CH₃ | D | H | H | H | D | H |
| 320 | CD₃ | CH₃ | D | H | H | D | D | D |
| 321 | CD₃ | CH₃ | D | H | H | D | H | D |
| 322 | CD₃ | CH₃ | D | H | H | H | D | D |
| 323 | CD₃ | CH₃ | D | H | D | D | D | H |
| 324 | CD₃ | CH₃ | D | H | D | D | H | H |
| 325 | CD₃ | CH₃ | D | H | D | H | D | H |
| 326 | CD₃ | CH₃ | D | H | D | D | D | D |
| 327 | CD₃ | CH₃ | D | H | D | D | H | D |
| 328 | CD₃ | CH₃ | D | H | D | H | D | D |
| 329 | CD₃ | CH₃ | D | D | H | D | D | H |
| 330 | CD₃ | CH₃ | D | D | H | D | H | H |
| 331 | CD₃ | CH₃ | D | D | H | H | D | H |
| 332 | CD₃ | CH₃ | D | D | H | D | D | D |
| 333 | CD₃ | CH₃ | D | D | H | D | H | D |
| 334 | CD₃ | CH₃ | D | D | H | H | D | D |
| 335 | CD₃ | CH₃ | D | D | D | D | D | H |
| 336 | CD₃ | CH₃ | D | D | D | D | H | H |
| 337 | CD₃ | CH₃ | D | D | D | H | D | H |
| 338 | CD₃ | CH₃ | D | D | D | D | D | D |
| 339 | CD₃ | CH₃ | D | D | D | D | H | D |
| 340 | CD₃ | CH₃ | D | D | D | H | D | D |
| 341 | CD₃ | CD₃ | H | H | H | D | D | H |
| 342 | CD₃ | CD₃ | H | H | H | D | H | H |
| 343 | CD₃ | CD₃ | H | H | H | H | D | H |
| 344 | CD₃ | CD₃ | H | H | H | D | D | D |
| 345 | CD₃ | CD₃ | H | H | H | D | H | D |
| 346 | CD₃ | CD₃ | H | H | H | H | D | D |
| 347 | CD₃ | CD₃ | H | H | D | D | D | H |
| 348 | CD₃ | CD₃ | H | H | D | D | H | H |
| 349 | CD₃ | CD₃ | H | H | D | H | D | H |
| 350 | CD₃ | CD₃ | H | H | D | D | D | D |
| 351 | CD₃ | CD₃ | H | H | D | D | H | D |
| 352 | CD₃ | CD₃ | H | H | D | H | D | D |
| 353 | CD₃ | CD₃ | H | D | H | D | D | H |
| 354 | CD₃ | CD₃ | H | D | H | D | H | H |
| 355 | CD₃ | CD₃ | H | D | H | H | D | H |
| 356 | CD₃ | CD₃ | H | D | H | D | D | D |
| 357 | CD₃ | CD₃ | H | D | H | D | H | D |
| 358 | CD₃ | CD₃ | H | D | H | H | D | D |
| 359 | CD₃ | CD₃ | H | D | D | D | D | H |
| 360 | CD₃ | CD₃ | H | D | D | D | H | H |
| 361 | CD₃ | CD₃ | H | D | D | H | D | H |
| 362 | CD₃ | CD₃ | H | D | D | D | D | D |
| 363 | CD₃ | CD₃ | H | D | D | D | H | D |
| 364 | CD₃ | CD₃ | H | D | D | H | D | D |
| 365 | CD₃ | CD₃ | D | H | H | D | D | H |
| 366 | CD₃ | CD₃ | D | H | H | D | H | H |
| 367 | CD₃ | CD₃ | D | H | H | H | D | H |
| 368 | CD₃ | CD₃ | D | H | H | D | D | D |
| 369 | CD₃ | CD₃ | D | H | H | D | H | D |
| 370 | CD₃ | CD₃ | D | H | H | H | D | D |
| 371 | CD₃ | CD₃ | D | H | D | D | D | H |
| 372 | CD₃ | CD₃ | D | H | D | D | H | H |
| 373 | CD₃ | CD₃ | D | H | D | H | D | H |
| 374 | CD₃ | CD₃ | D | H | D | D | D | D |
| 375 | CD₃ | CD₃ | D | H | D | D | H | D |
| 376 | CD₃ | CD₃ | D | H | D | H | D | D |
| 377 | CD₃ | CD₃ | D | D | H | D | D | H |
| 378 | CD₃ | CD₃ | D | D | H | D | H | H |
| 379 | CD₃ | CD₃ | D | D | H | H | D | H |
| 380 | CD₃ | CD₃ | D | D | H | D | D | D |
| 381 | CD₃ | CD₃ | D | D | H | D | H | D |
| 382 | CD₃ | CD₃ | D | D | H | H | D | D |
| 383 | CD₃ | CD₃ | D | D | D | D | D | H |
| 384 | CD₃ | CD₃ | D | D | D | D | H | H |
| 385 | CD₃ | CD₃ | D | D | D | H | D | H |
| 386 | CD₃ | CD₃ | D | D | D | D | D | D |
| 387 | CD₃ | CD₃ | D | D | D | D | H | D |
| 388 | CD₃ | CD₃ | D | D | D | H | D | D |

or a pharmaceutically acceptable salt of any of the foregoing, wherein any atom not designated as deuterium is present at its natural isotopic abundance.

17. The compound of claim 1, wherein X is —OH; Y^{1a} and Y^{1b} are the same; Y^{2a} and Y^{2b} are the same; Y^{3a} and Y^{3b} are the same; Y^{4a} and Y^{4b} are the same; and Y⁷, Y⁸, Y⁹, Y¹⁰, and Y¹¹ are each hydrogen; and the compound is selected from any one of the Compounds set forth in Table 4 below:
TABLE 4

| Compound # | R¹ | R² | Y^{1a}/Y^{1b} | Y^{2a}/Y^{2b} | Y^{3a}/Y^{3b} | Y^{4a}/Y^{4b} | Y⁵ | Y⁶ |
|---|---|---|---|---|---|---|---|---|
| 100 | CH₃ | CH₃ | H | H | H | H | H | D |
| 107 | CH₃ | CH₃ | D | H | H | H | H | H |
| 155 | CD₃ | CD₃ | D | H | H | H | H | H |
| 115 | CH₃ | CD₃ | H | H | H | H | H | H |
| 131 | CD₃ | CH₃ | H | H | H | H | H | H |
| 147 | CD₃ | CD₃ | H | H | H | H | H | H |
| 148 | CD₃ | CD₃ | H | H | H | H | H | D |
| 151 | CD₃ | CD₃ | H | D | H | H | H | H |
| 159 | CD₃ | CD₃ | D | D | H | H | H | H |
| 203 | CH₃ | CH₃ | H | H | D | D | D | H |
| 215 | CH₃ | CH₃ | H | D | D | D | D | H |
| 347 | CD₃ | CD₃ | H | H | D | D | D | H |
| 359 | CD₃ | CD₃ | H | D | D | D | D | H |
| 383 | CD₃ | CD₃ | D | D | D | D | D | H |

or a pharmaceutically acceptable salt of any of the foregoing, wherein any atom not designated as deuterium is present at its natural isotopic abundance.

18. The compound of any one of the preceding claims, wherein each position designated specifically as deuterium has at least 9000 incorporation of deuterium.

19. The compound of any one of the preceding claims, wherein each position designated specifically as deuterium has at least 95% incorporation of deuterium.

20. The compound of any one of the preceding claims, wherein each position designated specifically as deuterium has at least 97% incorporation of deuterium.

21. A pharmaceutical composition comprising a compound represented by structural formula (I): 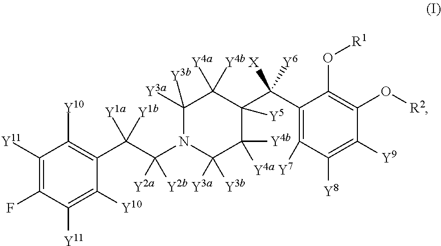
or a pharmaceutically acceptable salt thereof, wherein:
R¹ and R² are independently selected from —CH₃, —CH₂D, —CHD₂, and —CD₃;
X is —OH or —F; and
Y^{1a}, Y^{1b}, Y^{2a}, Y^{2b}, Y^{3a}, Y^{3b}, Y^{4a}, Y^{4b}, Y⁵, Y⁶, Y⁷, Y⁸, Y⁹, Y¹⁰, and Y¹¹ are each independently selected from hydrogen and deuterium;
provided that at least one of Y^{1a}, Y^{1b}, Y^{2a}, Y^{2b}, Y^{3a}, Y^{3b}, Y^{4a}, Y^{4b}, Y⁸, Y⁶, Y⁷, Y⁵, Y⁹, Y¹⁰, Y¹¹, R¹ and R² comprises deuterium; or
a compound of any one of claims 1-15, or a pharmaceutically acceptable salt thereof, and a pharmaceutically acceptable carrier.

22. A method of treating or preventing a disease or condition selected from psychosis, schizophrenia, schizoaffective disorder, Parkinson's disease, Lewy body dementia, sleep disorder, agitation, mood disorder, thromboembolic disorder, autism, attention deficit hyperactivity disorder, and any combination thereof, in a subject in need thereof, comprising administering to the subject an effective amount of a compound represented by structural formula (I): 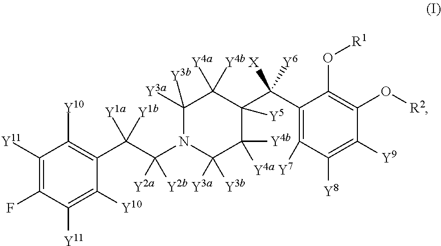
or a pharmaceutically acceptable salt thereof, wherein:
R¹ and R² are independently selected from —CH₃, —CH₂D, —CHD₂, and —CD₃;
X is —OH or —F; and
Y^{1a}, Y^{1b}, Y^{2a}, Y^{2b}, Y^{3a}, Y^{3b}, Y^{4a}, Y^{4b}, Y⁵, Y⁶, Y⁷, Y⁸, Y⁹, Y¹⁰, and Y¹¹ are each independently selected from hydrogen and deuterium;
provided that at least one of Y^{1a}, Y^{1b}, Y^{2a}, Y^{2b}, Y^{3a}, Y^{3b}, Y^{4a}, Y^{4b}, Y⁵, Y⁶, Y⁷, Y⁸, Y⁹, Y¹⁰, Y¹¹, R¹ and R² comprises deuterium;
a compound of any one of claims 1-15, or a pharmaceutically acceptable salt thereof, or
a pharmaceutical composition of claim 17.

23. The method of claim 22, wherein the subject is a human.

24. A pharmaceutical composition for treating or preventing a disease or condition selected from psychosis, schizophrenia, schizoaffective disorder, Parkinson's disease, Lewy body dementia, sleep disorder, agitation, mood disorder, thromboembolic disorder, autism, attention deficit hyperactivity disorder, and any combination thereof, comprising a compound represented by structural formula (I): 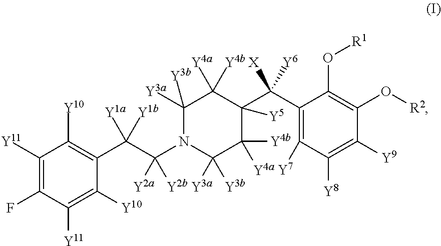
or a pharmaceutically acceptable salt thereof, wherein:
R¹ and R² are independently selected from —CH₃, —CH₂D, —CHD₂, and —CD₃;
X is —OH or —F; and
Y^{1a}, Y^{1b}, Y^{2a}, Y^{2b}, Y^{3a}, Y^{3b}, Y^{4a}, Y^{4b}, Y⁵, Y⁶, Y⁷, Y⁸, Y⁹, Y¹⁰, and Y¹¹ are each independently selected from hydrogen and deuterium;
provided that at least one of Y^{1a}, Y^{1b}, Y^{2a}, Y^{2b}, Y^{3a}, Y^{3b}, Y^{4a}, Y^{4b}, Y⁸, y, Y⁷, Y⁵, Y⁹, Y¹⁰, Y¹¹, R¹ and R² comprises deuterium; or a compound of any one of claims 1-15.

25. A compound represented by structural formula (I): 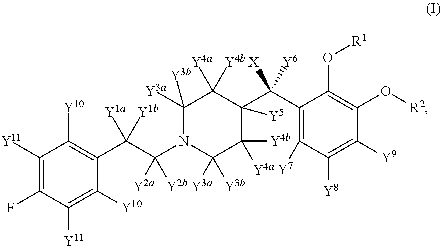
or a pharmaceutically acceptable salt thereof, wherein:
R¹ and R² are independently selected from —CH₃, —CH₂D, —CHD₂, and —CD₃;
X is —OH or —F; and
Y^{1a}, Y^{1b}, Y^{2a}, Y^{2b}, Y^{3a}, Y^{3b}, Y^{4a}, Y^{4b}, Y⁵, Y⁶, Y⁷, Y⁸, Y⁹, Y¹⁰, and Y¹¹ are each independently selected from hydrogen and deuterium;
provided that at least one of Y^{1a}, Y^{1b}, Y^{2a}, Y^{2b}, Y^{3a} Y^{3b}, Y^{4a}, Y^{4b}, Y⁶, Y⁷, Y⁸, Y⁹, Y¹⁰, Y¹¹, R¹ and R² comprises deuterium;
or a compound of any one of claims 1-15, or a pharmaceutically acceptable salt thereof, for use in treating or preventing a disease or condition selected from psychosis, schizophrenia, schizoaffective disorder, Parkinson's disease, Lewy body dementia, sleep disorder, agitation, mood disorder, thromboembolic disorder, autism, attention deficit hyperactivity disorder, and any combination thereof.

26. Use of a compound represented by structural formula (I): 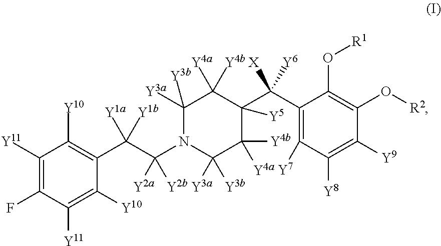
or a pharmaceutically acceptable salt thereof, wherein:
R¹ and R² are independently selected from —CH₃, —CH₂D, —CHD₂, and —CD₃;
X is —OH or —F; and
Y^{1a}, Y^{1b}, Y^{2a}, Y^{2b}, Y^{3a}, Y^{3b}, Y^{4a}, Y^{4b}, Y⁵, Y⁶, Y⁷, Y⁸, Y⁹, Y¹⁰, and Y¹¹ are each independently selected from hydrogen and deuterium;
provided that at least one of Y^{1a}, Y^{1b}, Y^{2a}, Y^{2b}, Y^{3a}, Y^{3b}, Y^{4a}, Y^{4b}, Y⁵, Y⁶, Y⁷, Y⁸, Y⁹, Y¹⁰, Y¹¹, R¹ and R² comprises deuterium; or
a compound of any one of claims 1-15, or a pharmaceutically acceptable salt thereof, for the manufacture of a medicament for treating or preventing a disease or condition selected from psychosis, schizophrenia, schizoaffective disorder, Parkinson's disease, Lewy body dementia, sleep disorder, agitation, mood disorder, thromboembolic disorder, autism, attention deficit hyperactivity disorder, and any combination thereof.
